(19) United States
(12) Patent Application Publication
(10) Pub. No.: US 2023/0390483 A1
(43) Pub. Date: Dec. 7, 2023

(54) APPARATUS AND METHODS FOR ADMINISTRATION OF MEDICINE INCLUDING MONITORING AND USER FEEDBACK

(71) Applicant: SHL MEDICAL AG, Zug (CH)

(72) Inventors: Martin Michael Coyne, III, Boonton, NJ (US); Christopher James Franzese, Boonton, NJ (US); Molly Christine Larson-Wakeman, Boonton, NJ (US); Alejandra Linares Martinez, Boonton, NJ (US); Matthew Conner D'Auria, Boonton, NJ (US); Katherine Ford Brigham, Boonton, NJ (US); Sebastian Karlsson, Zug (CH); Christoph Egloff, Zug (CH); Madeleine Hayden Anderson, Boonton, NJ (US)

(21) Appl. No.: 18/222,850

(22) Filed: Jul. 17, 2023

Related U.S. Application Data
(63) Continuation-in-part of application No. 18/119,982, filed on Mar. 10, 2023.
Application No. 18/119,982 is a continuation-in-part of application No. PCT/EP2022/071260, filed on Jul. 28, 2022.
(60) Provisional application No. 63/226,494, filed on Jul. 28, 2021.
(60) Provisional application No. 63/226,498, filed on Jul. 28, 2021.
(60) Provisional application No. 63/226,499, filed on Jul. 28, 2021.
(60) Provisional application No. 63/392,539, filed on Jul. 27, 2022.

(51) Int. Cl. A61M 5/142; A61M 39/08; A61M 5/158; A61M 5/168; G16H 20/17

(52) U.S. Cl. CPC A61M 5/142; A61M 39/08; A61M 5/158; A61M 5/16804; G16H 20/17; A61M 2039/082; A61M 2005/1588

(57) ABSTRACT

An apparatus including tubing sets, a modular constraint assembly, and methods of use are described for deliver a therapeutic medication to a patient, the apparatus can have a controller and a sensor. The controller is configured to receive data from the sensor, and to start and stop delivery of the therapeutic medication to the patient in response to data received from the sensor. In addition, apparatus, systems and methods are disclosed, which are configured to deliver a therapeutic medication to a patient. The apparatus, system and methods use a reservoir, a patient interface, a tubing set, a modular constraint assembly connected to the tubing sets, and a fluid pump, and the components are configured to provide a calibrated flow rate based upon specific characteristics of the therapeutic medications passing through and internal lumen of the tubing set.

### CROSS REFERENCE TO RELATED APPLICATIONS

The present application is a continuation-in-part of U.S. Ser. No. 18/119,982, filed Mar. 10, 2023, which is a continuation-in-part of WO2023/006907 (PCT/EP2022/071260) filed Jul. 28, 2022, which claims priority to U.S. Provisional Application No. 63/226,494, filed on 28 Jul. 2021; U.S. Provisional Application No. 63/226,498, filed on 28 Jul. 2021; U.S. Provisional Application No. 63/226,499, filed on 28 Jul. 2021, and U.S. Provisional Application No. 63/392,539, filed on 27 Jul. 2022, the contents of all of which are hereby incorporated by reference.

### TECHNICAL FIELD

Embodiments of the disclosure generally relate to apparatus, systems and methods for administration of therapeutic medicines. More particularly, embodiments of the disclosure relate to apparatus, systems and methods configured to power and/or monitor one or more sensors on a patient, the sensors configured to communicate with either a separate or integrated medication delivery apparatus and to communicate commands from a medication delivery apparatus to a needle assembly on a patient. In some embodiments of the disclosure, the apparatus may also be configured to provide a user of the system visual feedback on the device status.

Further embodiments of the disclosure generally relate to apparatus and methods for large volume infusion of therapeutic medicines. Specific embodiments of the disclosure pertain to apparatus and methods configured to deliver a one or more therapeutic medications to a patient at a known, preselected, and controlled flow rate.

### BACKGROUND

Infusion and injection are commonplace medical procedures used to administer a wide variety of therapeutic medicines of interest to treat a variety of diseases. As used herein, “infusion,” “injection,” and “administration” are used interchangeably, taking place by subcutaneous (SC), intramuscular (IM), intravenous (IV), or enteral routes, also terms used interchangeably. Liquid medication formulations are commonly infused and injected through a variety of needle assemblies, tubing sets, and fluid pumps through the intravenous, subcutaneous, and intramuscular routes.

Existing prefilled syringe (PFS) and autoinjector (AI) devices are suited for delivery of a small volume (≤2 mL) of a single biologic medicine, which are common treatments for chronic cardiovascular, gastrointestinal, autoimmune, immunologic, hematologic, endocrinology, and respiratory diseases. These medications are taken on a daily, weekly, biweekly, monthly, or quarterly basis. In general, a single medication is given, the dose does not change at each dosing interval, and the medications rarely provoke serious reactions in patients, facilitating straightforward administration at home. Thus, PFS and AI devices are designed for intuitive and rapid (˜10-15 sec) delivery of a single fixed-dose medicine by a user lacking clinical training, such as the patient themselves or a caregiver. While PFS and AI devices are well suited to simple low-volume monotherapies, novel delivery apparatus is needed to meet the needs of more complex clinical regimens.

Clinically, complex regimens are characterized by one or more aspects including, by way of example, large, delivered fluid volumes, numerous therapeutic medications, potentially variable dosing, medications given before and/or after a therapeutic medicine, and different sequences of medication administration. Administration may extend over a period of many hours and is currently performed in a hospital or clinic setting, overseen by a trained healthcare provider.

In a hospital or clinic setting, medication administration often includes supplemental monitoring of a patient's physiologic attributes using hospital-grade telemetry equipment that is separate from the drug delivery device and, like the delivery device, is durable equipment used for multiple patients. These physiologic parameters may include by way of example, one or more of heart rate, blood pressure, respiratory rate, blood oxygen saturation (SpO₂), and temperature. Healthcare providers monitor patient physiologic aspects before, during, and after medication administration to distinguish serious adverse events, such as a life-threatening systemic infusion reaction (SIR), from less side effects. Based on such data and clinical judgment, they must also make any appropriate changes to medication delivery. For example, if an SIR is detected by the clinician, they may stop medication delivery and administer one or more medications to treat the reaction. Thus, drug delivery devices for home administration of complex regimens should permit configuration to contingently administer emergency medications (emergency medicaments) at home based on physiologic data without a healthcare provider present, or with a clinically untrained user, such as a patient.

For home administration of complex medication regimens, it is obviously infeasible to routinely provide hospital-grade administration or patient monitoring equipment to every patient or place a clinician into each patient's home for many hours. Such equipment is bulky and expensive, requires clinical experience to safely operate, and relies upon clinical judgment for appropriate decisions related to safe medication administration. Untrained users, such as patients or caregivers at home, may be confused or overwhelmed by the multitude of data, settings, configurations, and connections with hospital grade administration or patient monitoring equipment; moreover, such confusion may lead to incorrect decisions or operation that may lead to situations that cause harm.

Thus, there is a need to provide improved drug delivery apparatus that allow safe home administration of more complex medication regimens, that allow intuitive use by those without clinical training, that provide patient monitoring capability to identify needed changes to medication administration without the presence of a healthcare provider, and that provide a user intuitive feedback about the status of administration.

For example, there is need to provide a drug delivery apparatus with medication reservoirs and a drive unit beside a patient or in a small bag, with only a small, lightweight needle assembly worn on the patient. The needle assembly may also include one or more sensors, or sensors may be separate from the needle assembly and attached to the patient. Such reservoirs and drive unit are fluidically connected to the needle assembly by a tubing set, the subject of the present disclosure.

Administration route is based on a specific medication's pharmacokinetic (PK) profile, formulation components, approved regulatory labeling, individual clinical judgment, or clinical necessity.

The SC or IM route is frequently used for administration of smaller volumes using prefilled syringes and autoinjectors. Biologic medicines are frequently administered via the SC route with these devices. However, medications with larger volumes are not suitable for these devices, and the IV route is typically chosen, generally in hospitals or outpatient clinics. Given the safety risks and patient burden of at-home IV administration, pharmaceutical companies and patients generally prefer at-home SC administration. SC administration is generally considered less invasive and more straightforward for patients. As physiologic uptake of medication is slower via the SC route, there is potential for improved tolerability compared to IV administration.

Given these significant advantages in safety, tolerability, and convenience, the pharmaceutical industry has invested heavily in transitioning formulations from IV to SC administration and medication administration from the clinic to the home setting. However, many large volume delivery devices such as syringe or volumetric pumps are intended for use only by trained healthcare professionals and are unsuitable for home use.

Ambulatory pumps for home use have been developed that provide an alternative to hospital-grade devices. However, they require configuration by a healthcare provider, aseptic assembly of components by patients, and may not work properly if specific components are unavailable or inadvertently substituted. These errors may lead to infection, medication errors, and serious adverse events. As a result, applicability of these devices is limited. To fully realize the benefits of large volume administration in the home setting, there is a need for simple, error-proof, safe, and intuitive delivery devices suitable for use by patients who are not trained healthcare providers.

While SC administration is highly preferred by pharmaceutical companies and patients, not all medicines are readily transitioned from IV-to-SC administration. Bioavailability is determined through in-human clinical trials, is molecule-specific, and generally lower for the SC route versus the IV route. For the same molecule, larger SC volumes are likely required to provide equivalent bioavailability compared to IV delivery. However, these volumes may exceed the capacity of current large-volume SC devices, such as on-body injectors (OBIs), which are supplied in fixed volume increments, such as 3 mL, 5 mL, 10 mL, 25 mL, and 50 mL. Should volume requirements exceed available OBI devices, or require customization of an OBI, follow-on clinical trials or commercial launch of medications using the OBI may be delayed.

Individual medications are often part of a larger regimen of medicines, with standardized regimens corresponding to a specific disease state, treatment regimen, or medication. In a clinic setting, order sets contain all the information required to administer a standardized regimen. For example, an oncology regimen might include pre-medications, oncology treatments, and post-medications, all contained in an order set. Existing drug delivery devices are designed to administer a single medication and cannot support delivery of multi-medication regimens, limiting the ability to move therapy from the clinic to the home setting. There are no delivery devices that can detect and respond to a suspected infusion reaction, making administration of certain medications currently infeasible in the home setting and confining these medications to in-clinic delivery.

Furthermore, medication order sets may direct clinical staff to perform specific patient monitoring and permit contingent administration of emergency medication. This is particularly important for medications that cause infusion-related reactions in certain patients. Infusion reactions are potentially fatal, systemic reactions related to mode of action of the medication. Systemic infusion reactions are clinically distinct from localized injection site reactions or erythema from administration of a single agent such as would occur with an autoinjector, prefilled syringe, or OBI device, which are uncomfortable but not life-threatening. They demand an immediate halt to medication administration and administration of one or more counteracting medications. However, prior art devices neither allow detection of systemic infusion reactions nor delivery of emergency medication and cannot be safely used to administer medications where systemic infusion reactions could occur. This is a particular concern for biologic therapies and is especially relevant to oncology treatments.

In the clinic setting, administration of a medication regimen, associated monitoring, and clinical decision-making are documented in the patient's record within an electronic health record (EHR) system. The purpose of the EHR is to provide a complete clinical record of care for a patient, and safely manage medication regimens without relying on human memory or introducing human error. Healthcare providers update and review the EHR system in real-time for a given patient. Current drug delivery devices for home use do not have EHR interfaces, preventing their use with multi-medication regimens, contingent medication administration, or specific patient monitoring requirements. Moreover, administration of medication via other drug delivery devices, such as OBIs, may not be reflected in an EHR system.

In the clinic setting, EHR systems also provide vital patient safety functions. EHR systems ensure patients may safely receive certain medications based on physical vital signs, laboratory testing values, or administration of prior medications as scheduled. However, prior art delivery devices used in the home setting are focused on a single medication, lack integration into EHR systems, and thus cannot provide safety interlocks that are present in the clinic. As a result, present devices cannot prevent administration of medications in unsafe conditions.

Accordingly, there is a need for allowing administration of other medications before, during, and after the therapeutic medication, even if outside the clinic setting. There is also a need for drug delivery systems which do not impose arbitrary volume restrictions or “breakpoints” upon the drug development process and decouple formulation development and clinical trials from delivery device, apparatus and system design. Furthermore, there is a need for drug delivery devices, apparatus and systems that are configured for detecting system infusion reactions through specific sensors, arresting delivery of a medicine, and administering one or more emergency counteracting medications. There is also a need for apparatus, systems and methods that provide EHR integration, advance the art of drug delivery devices, apparatus and systems by allowing home delivery of complex regimens as ordered, updating administration in a patient's record, and allowing healthcare providers to review a complete regimen history for a patient without extra effort. There is also a need to provide apparatus, systems and methods that allow integration with an EHR system and only allowing administration of medications under safe conditions, replicating the safety measures at home that are currently present in clinic settings.

### SUMMARY

Embodiments of the disclosure provide apparatus, systems and methods for administering large volumes of parenteral or enteral medicines to a patient via a tubing set. The apparatus or system enables different configurations of tubing sets to effectuate medication administration to a patient.

In one or more embodiments, the tubing sets described herein are used with a needle assembly configured with one or more sensors placed on a patient's skin, and a large volume medication delivery system that is located off the patient's body, containing one or more medications, a fluid pump, and a controller. Medication is delivered through one or more lumens provided in the tubing set, which fluidically connects the drug delivery device and patient needle assembly. Before, during, and after medication administration, the sensors allow the drug delivery system to monitor the physiologic status of a patient receiving the medication. In one or more embodiments, the tubing sets are also provided with a series of internally or externally situated conductors that enable electrical or optical communication between the needle assembly and a medication delivery device. In one or more embodiments, the conductors may comprise an electrical or optical conductor. In one or more embodiments, the externally situated conductors may comprise an electrically conductive printed ink.

One or more embodiments of the disclosure are directed to providing apparatus, systems and methods comprising communication systems and methods for drug delivery systems used in the home setting that are configured to communicate sensor data from the patient to the controller of a medication delivery system. One or more embodiments of the disclosure are directed to providing a more reliable, direct connection between a medication delivery system and a sensors within a needle assembly. In one or more embodiments, communication between the drug delivery system controller and needle assembly is used by the controller to measure a patient's physiologic status as reported by the sensor.

Additional embodiments of the disclosure are directed to providing apparatus, systems, and methods comprising powering systems and methods for drug delivery systems used in the home setting that are configured to power sensors located on a patient to allow continuous monitoring of physiologic parameters before, during, and after administration of one or more therapeutic medications. One or more embodiments of the disclosure are directed to providing drug delivery system configured with a controller with a more reliable, direct powering of sensors within a needle assembly. In one or more embodiments, communication between the drug delivery system controller and needle assembly is used to power the sensor in the needle assembly.

Further embodiments of the disclosure are directed to providing a medication delivery apparatus used for administration of complex medication regimens with a single feedback state as a consolidation of the system state, enabling simple, easily interpreted feedback to a user of the apparatus. In one or more embodiments, a drug delivery system controller is provided with an illuminated indicator in optical communication with a tubing set and is configured to provide a variety of visual feedback states to a user. An apparatus including a medication delivery device connected to a tubing set described herein may be provided with an illumination source proximal to the tubing set. When desired, the illumination source is piped through the tubing set or one or more optical conductors therein to use the tubing set itself as a feedback mechanism to the user. In some embodiments, use of single colors, combinations of colors, or flashing patterns may communicate one or more states to a user.

One or more embodiments of the disclosure are directed to an apparatus configured to deliver one or more therapeutic medications to a patient, the apparatus comprising a one or more reservoirs containing therapeutic medications; a patient interface configured to deliver contents of the reservoir into the body of the patient; a flexible tubing set in fluid communication with the reservoirs at the proximal end, and the patient interface at the distal end; and a fluid pump configured to expel the therapeutic medication from the reservoirs through the flexible tubing set and into the patient interface, wherein the flexible tubing set comprises a predetermined length and one or more internal medication lumens comprising a consistent internal diameter, the flexible tubing set configured to establish a specific, calibrated flow rate based on specific characteristics of the therapeutic medications passing through the internal lumen, the specific characteristics selected from the group consisting of viscosity, shear thinning behaviors, shear thickening behaviors, desired delivery time to the patient, and combinations thereof. In some embodiments the apparatus is modular. In some embodiments, the apparatus is configured to deliver the therapeutic medication to the patient at a known, preselected, and controlled flow rate. In some embodiments, the apparatus is configured to deliver the therapeutic medication to the patient at a known, preselected maximum flow rate. In some embodiments, the apparatus is configured to deliver a first medication at a known, preselected, and controlled first flow rate through a first lumen, and to deliver a second medication at a known, preselected, and controlled second flow rate through a second lumen, wherein the first flow rate is faster than the second flow rate.

Additional embodiments of the disclosure are directed to an apparatus configured to deliver a therapeutic medication to a patient, the comprising one or more reservoirs, each of the one or more reservoirs containing a therapeutic medication; one or more reservoirs containing a pre-medication to be administered before or a post-medication to be administered after the one or more therapeutic medications; a patient interface configured to expel contents of the reservoirs into the body of the patient; a flexible tubing set in fluid communication with the reservoirs at a proximal end of the flexible tubing set, and a patient interface at a distal end of the flexible tubing set; and a fluid pump to expel the therapeutic medication from each of the one or more reservoirs through the flexible tubing set and into the patient interface, wherein the flexible tubing set is provided with predetermined length and internal lumen of consistent internal diameter to provide a specific, calibrated flow rate based on characteristics of the therapeutic medications passing therethrough, the characteristics selected from the group consisting of viscosity, shear thinning behaviors, shear thickening behaviors, desired delivery time to the patient, and combinations thereof.

Further embodiments are directed to an apparatus configured to deliver one or more therapeutic medications to a patient, the apparatus comprising one or more reservoirs containing one or more therapeutic medications; an emergency reservoir containing an emergency medication; a patient interface configured to expel contents of the one or more reservoirs and the emergency reservoir into the body of the patient; and a flexible tubing set in fluid communication with the one or more reservoirs at a proximal end of the flexible tubing set, and the patient interface at a distal end of the flexible tubing set, wherein the flexible tubing set is provided with predetermined length and internal lumen of consistent internal diameter configured to provide a specific, calibrated flow rate based on characteristics of the therapeutic medications passing therethrough, the characteristics selected from the group consisting of viscosity, shear thinning behaviors, shear thickening behaviors, desired delivery time to the patient and combinations thereof.

Further embodiments are directed to an apparatus configured to deliver one or more investigational medicines during a clinical trial at one or more controlled flow rates, the apparatus comprising one or more reservoirs, each of the one or more reservoirs containing an investigational therapeutic medication; a patient interface configured to deliver contents of the reservoirs into the body of the patient; a flexible tubing set in fluid communication with the one or more reservoirs at a proximal end of the flexible tubing set, and the patient interface at a distal end of the flexible tubing set; and a fluid pump configured expel the investigational therapeutic medication from the reservoir through the flexible tubing set and into the patient interface, wherein each of several the flexible tubing sets is provided with a predetermined length and an internal lumen of a consistent internal diameter to provide a specific, calibrated flow rate based on characteristics of the investigational therapeutic medications passing therethrough, the characteristics selected from the group consisting of dose, concentration, viscosity, shear thinning behaviors, shear thickening behaviors, desired delivery time to the patient and combinations thereof, the characteristics corresponding to one or more clinical trial study conditions.

Another aspect of the disclosure is directed to a method for delivering an investigational therapeutic medication to a patient at one or more controlled flow rates during a clinical trial of an investigational medicine, the method comprising providing a clinical trial kit comprising an investigational therapeutic medication, a reservoir, a fluid pump, and one or more flexible tubing sets, each of the one or more flexible tubing set corresponding to a specific controlled flow rate for a specific investigational therapeutic medication and associated with one or more clinical trial conditions; selecting a selected flexible tubing set from the one or more flexible tubing sets corresponding to an individual patient's clinical trial condition, as specified in a clinical trial protocol or randomization schedule; attaching a proximal end of the flexible tubing set to the fluid pump to establish fluid communication with the fluid pump; attaching a distal end of the flexible tubing set to a patient interface; and administering an investigational therapeutic medication to the patient.

In another embodiment of a method, a method of providing an optimized tubing set for delivery to a patient a therapeutic medication exhibiting substantially non-Newtonian characteristics delivered by a single pump unit at one or more known, preselected, and controlled flow rates is provided. The method comprises identifying one or more desired flow rates of the therapeutic medication for administration to a patient based on desired pharmacokinetics of the therapeutic medication; identifying one or more ambient temperatures at which delivery of the therapeutic medication will occur; conducting testing to identify a relationship between temperature, viscosity, and concentration of the therapeutic medication in a pharmaceutical formulation for delivery to the patient; specifying values of an internal diameter, a length, and interior surface roughness of an experimental tubing set associated with one or more of the desired flow rates, based on one or more of theoretical calculations and computational fluid dynamic analysis; characterizing a force required to propel the therapeutic medication exhibiting non-Newtonian characteristics through the experimental tubing set; experimentally determining a required fluid pump power to dispense the therapeutic medication within the experimental tubing set at a plurality of temperatures and flow rates; adjusting the values of the experimental tubing set to accommodate an observed flow rate versus a desired flow rate and selecting the optimized tubing set; and confirming the desired flow rate through the optimized tubing set.

Further aspects concern tubing sets and apparatus comprising these tubing sets, as set out in more detail in the description below.

### BRIEF DESCRIPTION OF THE FIGURES

FIG. 1A shows a simplified partial cutaway front view diagram showing anatomic location of patient interface components to effectuate intravenous medication delivery using four common vascular access devices (VADs) featuring terminating luer taper connections in accordance with one or more embodiments;

FIG. 1B shows a simplified partial cutaway front view diagram showing anatomic location of patient interface components to effectuate intravenous medication delivery using an implanted vascular access device (VADs) or “port” and Huber needles in accordance with one or more embodiments;

FIG. 1C shows a simplified partial cutaway front view diagram showing anatomic location of patient interface components to effectuate subcutaneous and intramuscular administration using a variety of straight in and angled needle placements in accordance with one or more embodiments;

FIG. 1D shows a simplified partial cutaway front view diagram showing anatomic location of patient interface components to effectuate placement of a soft, flexible administration cannula and provide subcutaneous and intramuscular administration in accordance with one or more embodiments;

FIG. 2A shows a block diagram of selected functional components implemented in the drug delivery apparatus to deliver three medications in accordance with one or more embodiments;

FIGS. 2B-1 through 2B-5 show block diagrams of selected functional components implemented in the drug delivery apparatus to deliver combination therapy of several medications, illustrating exemplary administration sequences and time-delays based on regimen requirements, in accordance with one or more embodiments;

FIG. 2C shows a block diagram of selected functional components implemented in the drug delivery apparatus to deliver a therapeutic medication preceded and/or succeeded by certain other medications as part of a full medication regimen in accordance with one or more embodiments;

FIG. 2D shows a block diagram of selected functional components implemented in the drug delivery apparatus to deliver a medication of interest as well as various flushing solutions in accordance with one or more embodiments;

FIG. 2E shows a block diagram of selected functional components implemented in the drug delivery apparatus to deliver a medication of interest and contingently administer an emergency medication to counteract a systemic infusion reaction in accordance with one or more embodiments.

FIG. 3A shows a flow diagram of a clinical trial study process, illustrating how the present disclosure is integrated therein in accordance with one or more embodiments.

FIG. 3B shows a flow diagram of a process within one embodiment to design and refine a tubing set to deliver a non-Newtonian therapeutic medication at one or more rates based on formulation characteristics, expected pharmacotherapeutic effect, and expected dosing regimens studied as part of a human clinical trial.

FIG. 3C shows a schematic diagram of the governing parameters to design and refine a tubing set to deliver a substantially non-Newtonian therapeutic medication given formulation characteristics in accordance with one or more embodiments.

FIG. 4 shows a block diagram of selected functional components implemented in the drug delivery apparatus to provide closed-loop monitoring of patient status in order to detect a systemic infusion reaction and allow one or more appropriate clinical responses to the systemic infusion reaction in accordance with one or more embodiments.

FIG. 5 shows a flow diagram of a process of one embodiment for detecting and responding to a patient infusion reaction during or after administration of one or more therapeutic medications.

FIGS. 6A-C show a cross-sectional diagram of tubing sets and medication lumens in accordance with one or more embodiments.

FIGS. 7A-B illustrate a portion of a tubing set containing an inline filter and flow restrictor in accordance with one or more embodiments.

FIG. 8 shows a block diagram of selected functional components implemented in the drug delivery apparatus to provide clinical study data integrity for an investigational therapeutic medication in accordance with one or more embodiments.

FIG. 9 shows a block diagram of selected functional components to deliver one or more therapeutic medications to a patient.

FIG. 10A is a representative example of information in a medication order for a single medication contained within an electronic health record system.

FIG. 10B is a representative example of information in a medication order set contained within an electronic health record system for administration of a medication regimen, including a variety of medication administration and other care instructions for a patient.

FIG. 10C shows a block diagram of selected functional components implemented in the drug delivery apparatus to provide association and verification of a drug delivery apparatus against a medication order in accordance with one or more embodiments.

FIG. 11 shows a block diagram of selected functional components of a medication delivery apparatus or system incorporating the tubing set described herein in one or more embodiments.

FIGS. 12A-I show cross-sectional views of selected tubing sets and conductors in accordance with one or more embodiments.

FIG. 13 shows a schematic view of tubing sets providing visual feedback in accordance with one or more embodiments.

FIG. 14A shows a partial cutaway side view of a constraint being oriented to a tubing set in accordance with an embodiment of the disclosure.

FIG. 14B shows a partial cutaway side view of a constraint placed on a tubing set in accordance with an embodiment of the disclosure.

FIG. 15A shows a cross-sectional view of an adjustable constraint apparatus, configured to provide multiple discrete degrees of constraint to a tubing set in accordance with an embodiment of the disclosure.

FIG. 15B shows an end view of an adjustable constraint apparatus, configured to provide multiple discrete degrees of constraint to a tubing set in accordance with an embodiment of the disclosure.

FIG. 15C shows an end and cross-sectional view of an adjustable constraint apparatus, illustrating progressive assembly of the device through three levels of successively larger constraint to a tubing set in accordance with an embodiment of the disclosure.

FIG. 16A shows a perspective exploded view of an adjustable constraint apparatus provided with an emergency tubing set shutoff clamp and configured to provide multiple discrete degrees of constraint to a tubing set in accordance with an embodiment of the disclosure.

FIG. 16B shows a perspective view of the assembled components of FIG. 16A and the shutoff clamp in a first of two discrete positions.

FIG. 16C shows a perspective view of the assembled components of FIG. 16A and the shutoff clamp in a second of two discrete positions.

FIG. 17A shows an end view of several constraints assembled onto tubing sets, illustrating a variety of shapes in accordance with one or more embodiments of the disclosure.

FIG. 17B shows a top view of several constraints assembled onto tubing sets and visible indicia after assembly, in accordance with one or more embodiments of the disclosure.

FIG. 17C shows a side view, top view, and bottom view of a constraint assembled onto a tubing set and visible and machine readable indicia after assembly, in accordance with one or more embodiments of the disclosure.

FIGS. 18A, 18B, 18C, 18D, 18E, 18F, 18G, 18H, 19A, 19B, 19C, 20A and 20B show various solutions for compression of a tube.

FIG. 21 shows two configurations of a medicament delivery device.

FIGS. 22 and 23 shows example configurations of a powerpack.

FIGS. 24A, 24B, 25A, 25B and 26 show example tubing set configurations.

FIG. 27 shows an example tubing structure.

FIG. 28 shows a top view of two possible modular constraint assemblies having visible indicia and that are removably connected to a tubing set in accordance with an embodiment of the disclosure.

FIG. 29 shows a partial cutaway side view of a possible modular constraint assembly removably connected to a tubing set in accordance with an embodiment of the disclosure.

FIGS. 30A & 30B show two possible keyed connectors that can be used with the modular constraint assemblies of FIGS. 28 and 29 in accordance with an embodiment of the disclosure.

FIG. 31 is a schematic process diagram for one possible IR determination and prediction system.

FIG. 32 is a first part of a schematic process diagram for the system of FIG. 31.

FIG. 33 is a second (continuation) part of the schematic process diagram of the system of FIG. 31.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

The design and use of large volume medication delivery apparatus and systems raise new challenges. The characteristics of medications are often different than those administered with existing devices. Some medications administered in larger volumes may trigger systemic infusion reactions during or after administration. These infusion reactions are potentially fatal, systemic reactions related to mode of action of the medication and are distinct from localized injection site reactions or erythema from administration of a single agent with a prefilled syringe, autoinjector, or on-body injector, which are uncomfortable but not life-threatening. Such apparatus and systems may require an immediate halt to medication administration and administration of one or more counteracting emergency medications, for example, epinephrine.

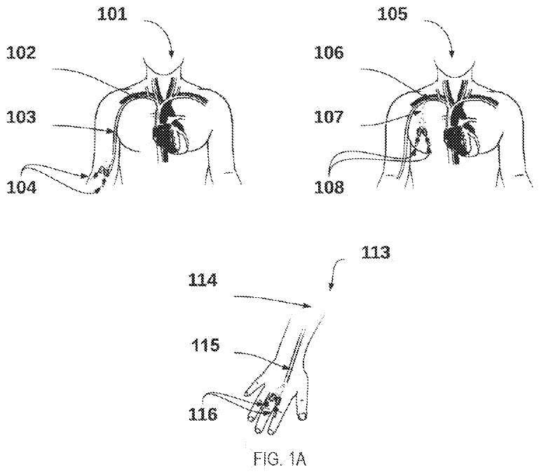

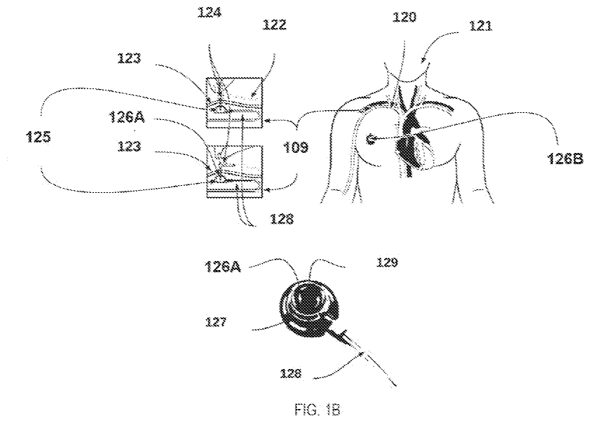

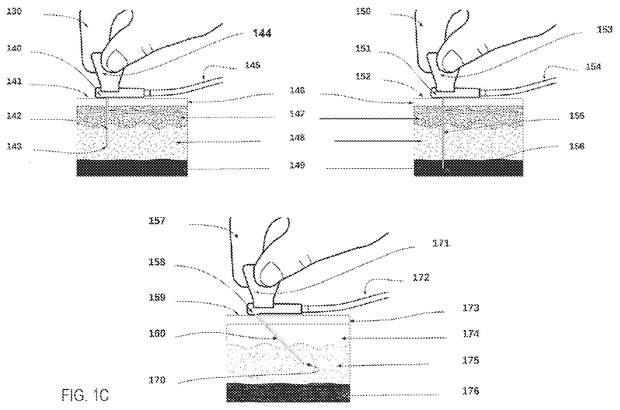

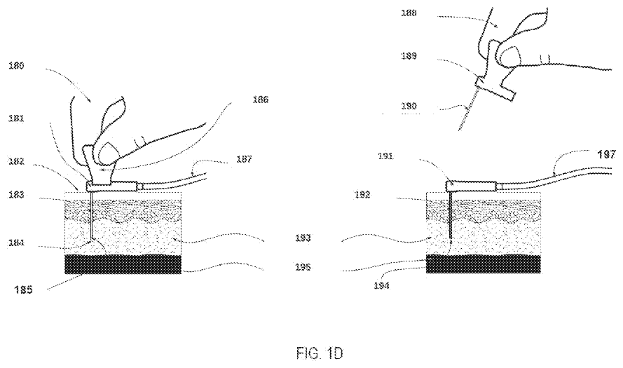

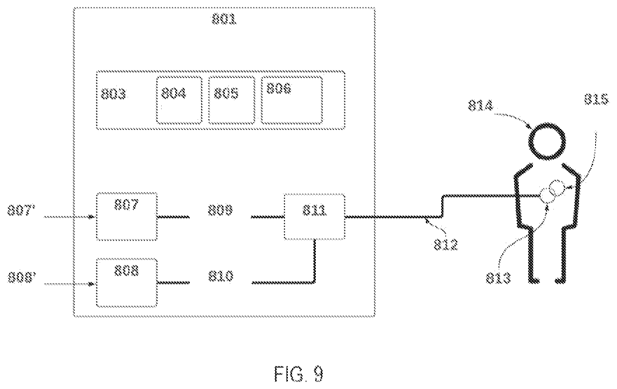

Each of the embodiments described with respect to FIGS. 11-13 may be combined with each of the embodiments described in FIGS. 1-10C and the corresponding claims.

FIG. 11 illustrates an exemplary large volume medication delivery system comprising an outer housing 1120, and one or more reservoirs 1122′ for one or more therapeutic medication(s) 1122 fluidically connected 1123 to a fluid pump 1124, by which the reservoirs 1122′ may be emptied by the fluid pump 1124 to administer the medication 1122 to the patient 1128 by way of the tubing set 1125 disclosed herein and patient interface 1126.

The medication delivery system is also provided with a controller 1121 that communicates with components of the apparatus via either a wired or wireless connection. In one or more embodiments, the controller according to one or more embodiments comprises a processor 1121a, a memory 1121b coupled to the processor 1121a, input/output devices 1121c coupled to the processor 1121a, and support circuits to provide communication between the different components of the system, namely the components of the system described herein. In one or more embodiments, processes to operate the system are stored in the memory 1121b as a software routine that, when executed by the processor, causes the system to perform methods described in the present disclosure. In one or more embodiments, the processes to operate the system are performed in hardware. In one or more embodiments, the software routine to operate the system may also be stored and/or executed by a second processor that is remotely located from the hardware being controlled by the processor.

In one or more embodiments, the tubing set 1125 is provided with one or more medication lumens 1125f by which medication 1122 is instilled by fluid pump 1124 into the patient 1128 through the patient interface 1126. Said patient interface 1126 can comprise a luer-taper or Luer-Lok® fitting if a luer-activated IV connector is used, or a variety of needle assemblies corresponding to an intravenous, subcutaneous, or intramuscular route. Sensors 1127 are located on a patient, either separately or integral to the patient interface 1126. In one embodiment, patient interface 1126 may comprise a subcutaneous, intramuscular, or intravenous needle set with integral sensors 1127.

In one or more embodiments, tubing set 1125 is also provided with one more conductors 1129 by which the sensors 1127 are coupled to the controller 1121. In one or more embodiments, the tubing set 1125 is also provided with an optical conductor 1125o which may be used to provide one or more visual feedback indicators to a patient 1128 or another user of the device.

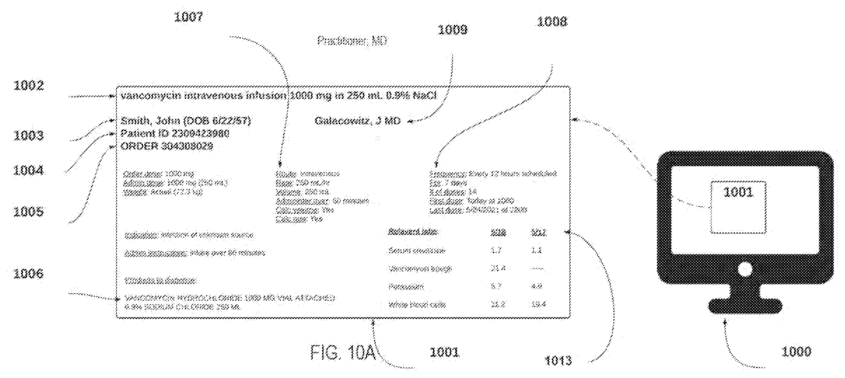

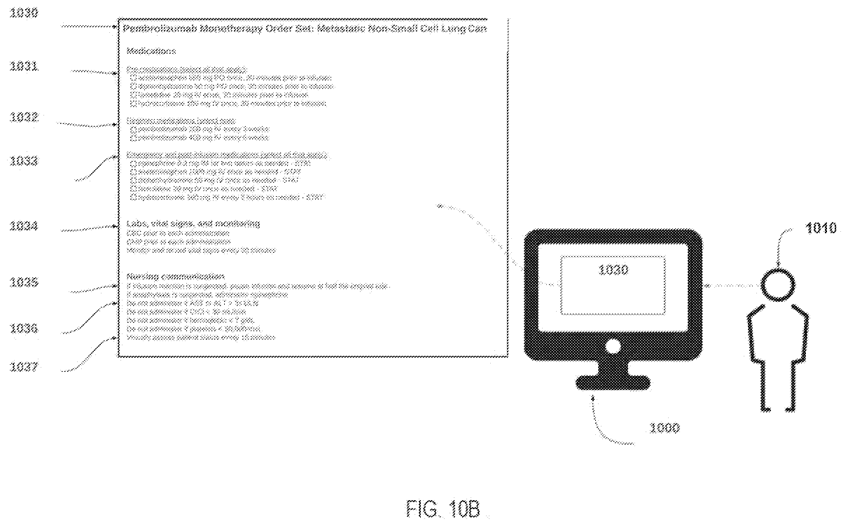

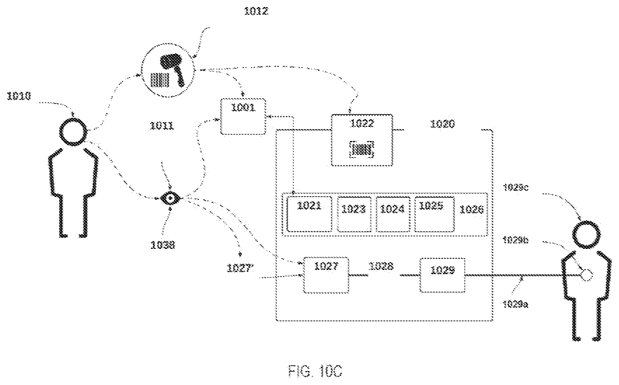

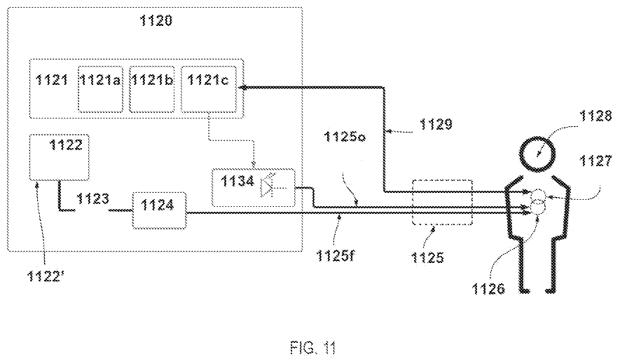

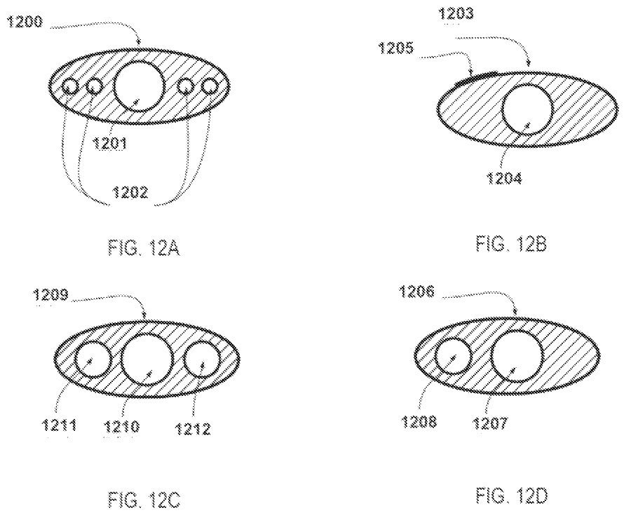

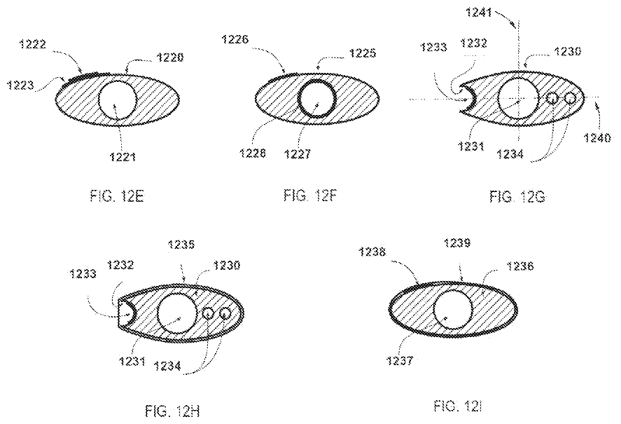

FIGS. 12A-I illustrate exemplary embodiments of medication lumens and conductors according to one or more embodiments. Although certain arrangements of medication lumens and conductors are depicted in FIGS. 12A-I, it will be apparent to those skilled in the art that many arrangements of medication lumens and conductors are within the scope of the present disclosure, and that the illustrative figures provided are for purposes of illustration and shall not limit the arrangements of conductors and lumens.

As seen in FIGS. 12A-I, in one or more alternative embodiments, a tubing set 1200 is provided with one or more medication lumens 1201 and one or more internal conductors 1202. In one or more alternative embodiments, a tubing set 1203 is provided with one or more internal medication administration lumens 1204 and one or more external conductors 1205, wherein the one or more conductors 1205 are substantially situated on the exterior of the tubing 1203. Said conductors 1202, 1205 enable coupling and communication between a controller and one or more sensor(s), while said lumens 1201, 1204 enable medication administration.

Conductors 1202, 1205 are situated in a parallel manner to medication lumens 1201, 1204 over the length of the tubing set, which may be varied based on the delivery devices and patient interfaces used.

In one or more alternative embodiments, a tubing set 1206 is provided with one or more internal medication lumens 1207 and one or more internal optical conductors 1208. Optical conductors 1208 may be used as described herein to provide visual feedback to one or more users of a medication delivery device incorporating tubing set 1206.

In one or more embodiments, a tubing set 1209 is provided with one or more internal medication lumens 1210, one or more internal optical conductors 1211, and one or more conductors 1212. Optical conductors 1211 may be used to provide visual feedback to one or more users of a medication delivery device, while conductors 1212 may be used to communicate sensor data from a patient interface to a medication delivery system controller, both as described herein. incorporating tubing set 1206. In an alternative embodiment, conductors 1212 are used to power sensors within the patient interface.

In an alternative embodiment, a tubing set 1220 with one or more external conductors 1222 is provided with an interposing barrier coating 1223 to isolate medication within one or more fluid lumens 1221 from potential contaminant leachable or extractable compounds from the conductors 1222 or the conductor application process.

In an alternative embodiment, a tubing set 1225 with one or more external conductors 1226 and one or more medication lumens 1227 is provided with an interposing barrier coating 1228 situated between the medication lumen 1227 and tubing set 1225 material, to isolate medication within one or more fluid lumens 1221 from potential contaminant leachable or extractable compounds from the external conductors 1226 or the conductor application process. In one embodiment, the barrier coating 1223, 1228 comprises a PTFE or other fluoropolymer material. In another embodiment, the barrier coating 1223, 1228 is co-extruded as the tubing set is manufactured.

Referring to FIGS. 12G and 12H, in one or more alternative embodiments, a tubing set 1230 is provided with one or more internal medication lumens 1231, an undercut 1232, and one or more external conductors 1233 situated in said undercut 1232, to protect said external conductors 1233 from damage, for example from chafing or friction. In some embodiments, one or more undercuts 1232 may be provided in the tubing set 1230 to accommodate and protect additional external conductors 1233. In some embodiments, the tubing set 1230 may have a combination of one or more external 1233 and internal conductors 1234. The undercut is a recess (for example a groove).

In some embodiments, the tubing set 1230 comprises an asymmetric cross-section with differing bending stiffness about a first axis 1240 and a second axis 1241 orthogonal to the first axis 1240. In some embodiments, one or more undercut features is 1232 situated on an exterior contour of the tubing set 1230 and oriented on a cross-sectional axis with a higher degree of bending stiffness to protect said external conductors 1233 from damage, for example from bending, fatigue, or stress cracking.

In some embodiments, the tubing set 1230 may be provided with an outer protective sheath 1235 substantially enclosing the undercut 1232 and part or all of the outer surface of the tubing set 1230 to protect said conductors from damage, such as by friction or chafing. In some embodiments, the material of tubing set 1230 has a lower flexural stiffness compared to that of the protective sheath 1235 protecting the external 1233 and or internal conductors 1234 from abrasion and damage due to excessive bending. In some embodiments, the tubing set 1236 may be provided with one or more external conductors 1238 and an outer protective sheath 1239 covering part or all of the outer surface of the tubing set 1236 to protect said external conductors 1238 from damage, such as by friction or chafing. In one embodiment, protective sheaths 1235, 1239 are applied after manufacturing of the tubing set and application of one or more external conductors 1233, 1238. In one embodiment, protective sheaths 1235, 1239 comprise a textile material to provide a patient with additional privacy or discretion during medication administration.

The tubing set and barrier coating(s), if present, may be fashioned from one or more of silicone, PVC, PVC without DEHP, EVA, HDPE, LDPE, TPU, PTFE, fluoropolymer, or other suitable flexible material. In some embodiments, the tubing set is manufactured from a flexible polymer that is an electrical insulator. Tubing sets of some embodiments are extruded but may be formed by other means that provide sufficient dimensional and tolerance control on the inner medication lumens as described herein. In one or more embodiments, the tubing material is chosen to be a material selected for low leachable and extractable compounds that may contaminate a medication, and that exhibits high biocompatibility with biologic medications.

The tubing set and barrier coating(s), if present, may be fashioned from one or more of silicone, COC, COP, PVC, PVC without DEHP, EVA, HDPE, LDPE, TPU, PTFE, PCTFE, fluoropolymer, or other suitable flexible material. In some embodiments, one or more tie layers may be provided to allow bonding of two or more materials comprising the tubing set, such as a drug contacting material and a barrier coating. In some embodiments, the tubing set is manufactured from a flexible polymer that is an electrical insulator. Tubing sets of some embodiments are extruded but may be formed by other means that provide sufficient dimensional and tolerance control on the inner medication lumens as described herein. In one or more embodiments, the tubing material is chosen to be a material selected for low leachable and extractable compounds that may contaminate a medication, and that exhibits high biocompatibility with biologic medications. In one or more embodiments, the tubing set material comprises a COC drug contacting layer on the inner side and PCTFE film (for example from Aclar) on the outer side, bonded through an intermediary tie layer, as shown in FIG. 27. The 160-micron thickness as shown in FIG. 27 is exemplary, and could be varied.

In some embodiments, one or more conductors are manufactured from an electrical conductor, such as carbon, copper, nickel, or silver. In some embodiments, the conductors comprise a conductive ink. In one or more embodiments, the external conductors comprise a conductive ink. The material comprising the conductive ink may include a solution of metal nanoparticles. The conductive ink may be applied to tubing set during manufacture of the tubing or as a separate secondary operation. The conductive ink may be applied by flexographic, screen, inkjet, or stencil printing. In one or more alternative embodiments, the externally situated conductors comprise a conductive polymer. In one or more alternative embodiments, the conductive polymer is selected from one or more of polyacetylene, polythiophene, poly[3,4-(ethylenedioxy)thiophene], polypyrrole, polyaniline, or polyphenylene. In one or more alternative embodiments, the externally situated conductors 654 comprise an electrodeposited film. In one or more embodiments, the electrodeposited film is a polypyrrole-polyaniline composite conductive film.

In some embodiments, one or more conductors are optical conductors, comprising an optical fiber, silica fiber bundle, polymer, or flexible polymer tube and liquid core combination. In some embodiments, one or more conductors are optical conductors that can transmit light wavelengths outside the human visible spectrum. In some embodiments, one or more conductors are optical conductors that can transmit light wavelengths within the human visible spectrum.

In some embodiments, the tubing sets are sterilized using a low-energy method, such as ethylene oxide gas or vaporized hydrogen peroxide, to preserve electrical or optical conductor continuity. In some embodiments, the tubing sets are sterilized using a high-energy method, such as gamma irradiation or electron beam irradiation, configured in a manner to preserve electrical or optical conductor continuity.

In one or more embodiments of the disclosure herein, tubing sets may also be enabled to provide feedback to a user on the status of the medication delivery system. Feedback to the user may include, for example, confirmation of proper setup, readiness to administer medications, progress of one or more medication administrations, an error in configuration prior to administration, an error during administration of one or more medications, or completion of medication administration. Those skilled in the art will appreciate the wide variety of user feedback that may be provided to one or more users of a medication delivery system based on the user population, medication regimen, and clinical characteristics.

In one or more embodiments of the disclosure herein, tubing sets may also be enabled to provide feedback to a user on the status of the medication delivery system. Feedback to the user may include, for example, confirmation of proper setup, readiness to administer medications, progress of one or more medication administrations, an error in configuration prior to administration, an error during administration of one or more medications, or completion of medication administration. Feedback to a user may include start of dose, end of dose, or in-process feedback, as is customary with autoinjector devices, or may be specific to the nature or configuration of the device. For instance, in a device with multiple needle sets or tubing lumens, feedback related to confirmation of proper setup or readiness to administer medications may comprise an indicator of which needle set should be inserted and in which order, helping a user identify the appropriate needle set among many. Continuing this example, feedback related to completion of medication administration may comprise feedback indicating which needle is ready to be removed amongst a plurality of needles. Alternatively, in a device with multiple medication reservoirs, feedback related to confirmation of proper setup or readiness to administer medications may comprise an indicator of proper bag installation (i.e., fluidic and/or sensor communication) within the device. Also in the alternative, in a device with multiple needles or tubing set lumens, feedback related to confirmation of proper setup or readiness to administer medications may comprise an indicator of proper needle insertion. Feedback related to confirmation of proper setup may include, by way of example, an indication that multiple segments of an infusion set are properly connected (i.e., fluidically, electrically, optically, pneumatically), or that a needle set is properly connected to a tubing set and/or tubing set lumen. Feedback related to an error during administration of one or more medications may include, by way of example, an occlusion in the tubing set or needle, excessive (i.e., unintended, unprescribed, or unsafe) flow rate, or removal of a needle from the skin during injection. Those skilled in the art will appreciate the wide variety of user feedback that may be provided to one or more users of a medication delivery system based on the user population, medication regimen, drug delivery device configuration, and clinical characteristics.

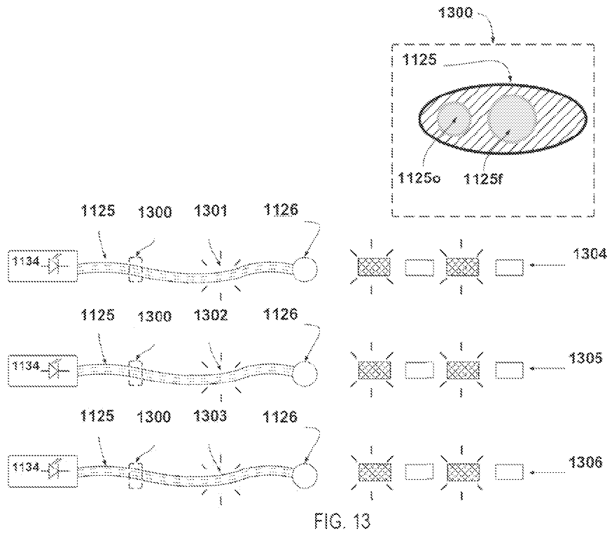

In an alternative embodiment, referring to FIG. 13, in one or more alternative embodiments of the medication delivery system, a tubing set 1125 is provided with a cross-sectional design 1300 featuring an optical conductor 1125o and a medication lumen 1125f. The tubing set 1125 is positioned so as to make optical contact with a selectively illuminated indicator 1134, housed in the medication delivery system as described elsewhere herein. When indicator 1134 is illuminated, as by a controller, light is conducted through the optical conductor 1125o in tubing 1125, enabling visual feedback 1301 over the length of the tubing set 1125 or a portion thereof.

In some embodiments, said indicator 1134 is an addressable LED which can display a plurality of different colors, enabling a variety of visual feedback states to be communicated to an end user. In some embodiments, the visual feedback comprises a visible indicator of differently colored lights 1301, 1302, 1303. In some embodiments, the visual feedback comprises a visible indicator of green, amber, and red lights 1301, 1302, 1303. In some embodiments, the visual feedback comprises a pulsed visual indicator of differently colored lights 1304, 1305, 1306. In another alternative embodiment, the visual feedback comprises a pulsed visual indicator of green 1304, amber 1305, or red 1306.

In some embodiments, intermittent light signals of color and/or flashing patterns (red flashing, red-yellow pulsing, or fast or slow flashing) may be used in place of different colors 1301, 1302, 1303. In another alternative embodiment, said indicator 1134 is pulsed in one or more feedback patterns 1304, 1305, and 1306, wherein the feedback patterns are independent of the indicator 1134 color.

In another alternative embodiment, the tubing 1125 is selectively masked in an opaque color, hiding and/or revealing one or more portions of light conducted through the optical conductor 1125o, and enabling further permutations of visual feedback to be presented to the end user. In another alternative embodiment, the tubing 1125 is selectively covered in an opaque or translucent textile material to protect a patient's privacy or provide additional discretion during medication administration.

In one or more embodiments, the tubing sets described immediately above with respect to FIGS. 11-13 are configured to be used or combined with the apparatus, systems and methods described with respect to FIGS. 1-10 and the embodiments described with respect to FIGS. 1-10 described below (apparatus and method for large volume medication administration). Thus, in some embodiments, the apparatus, systems and methods described with respect to FIGS. 1-10 further comprise the apparatus, tubing sets, methods and kits described immediately above with respect to FIGS. 11-13 in combination or addition to the various embodiments described below, including but not limited to the first 126 numbered embodiments.

As used herein, “infusion,” “injection,” and “administration” are used interchangeably, taking place by subcutaneous (SC), intramuscular (IM), intravenous (IV), or enteral routes, also terms used interchangeably. Administration route is based on a specific medication's pharmacokinetic (PK) profile, formulation components, approved regulatory labeling, individual clinical judgment, or clinical necessity.

Embodiments of the disclosure provide apparatus, system and methods for medication administration wherein the number of medications, administration order, volume, delivery time, and route of administration are independently selected. Embodiments of the apparatus, systems and methods provide a single architecture usable from initial human clinical trials in a research facility through commercial launch in a home setting after drug approval. One or more embodiments provide for use in the home setting, where the apparatus, systems and methods are intrinsically safe and intuitive for use by a patient or lay caregiver without healthcare training.

Accordingly, embodiments of the disclosure provide drug delivery apparatus, systems and methods allowing delivery of many different medications, including those historically limited to in-clinic settings, in the home in a variety of sequences, rates, and settings. As will be appreciated by one skilled in the art, there are numerous ways of carrying out the examples, improvements and arrangements of devices, apparatus and/or systems disclosed herein. Although reference will be made to the exemplary embodiments depicted in the drawings and the following descriptions, the embodiments disclosed herein are not meant to be exhaustive of the various alternative designs and embodiments that are encompassed by the present disclosure.

Embodiments of the present disclosure advance the art of drug delivery devices, apparatus or systems by allowing administration of other medications before, during, and after the therapeutic medication, even if outside the clinic setting. One or more embodiments of the disclosure do not impose arbitrary volume restrictions or “breakpoints” upon the drug development process and decouple formulation development and clinical trials from delivery device, apparatus or system design. In addition, embodiments advance drug delivery devices, apparatus or systems by allowing detection of system infusion reactions through specific sensors, arresting delivery of a medicine, and administering one or more emergency counteracting medications. One or more embodiments of the disclosure further provide apparatus, systems and methods that provide EHR integration, advance the art of drug delivery devices, apparatus or systems by allowing home delivery of complex regimens as ordered, updating administration in a patient's record, and allowing healthcare providers to review a complete regimen history for a patient without extra effort. One or more embodiments provide apparatus, systems and methods that allow integration with an EHR system and only allowing administration of medications under safe conditions, replicating the safety measures at home that are currently present in clinic settings.

Various embodiments of the disclosure are directed to improved systems or apparatus and methods configured for large volume infusion of therapeutic medicines. More particularly, embodiments provide systems, apparatus and methods comprising components configured to be combined to deliver one or more therapeutic medicines via one or more physiologic routes of administration in sufficiently large and varying volumes to achieve a desired therapeutic effect. In one or more embodiments, therapeutic medicines may also optionally include pre-, post- and emergency medication administration to effectuate a complete therapeutic regimen as ordered by a healthcare professional. In some embodiments, the components utilized are part of a kit, and may be referred to as a kit of components. The systems, apparatus and methods of one or more embodiments are used to determine the pharmacologic and physiologic effects of one or more therapeutic medicines when the characteristics are unknown, and may be then used to deliver the therapeutic medicine(s) at the desired parameters to achieve the therapeutic effect when administered in a variety of settings, such as in-clinic or at-home. In addition, the system, apparatus and methods of one or more embodiments improve usability, safety, and convenience based on the administration setting and end user of the drug delivery device, apparatus or system.

One of more embodiments of the disclosure provides new and/or improved apparatus, systems, and methods for administering large volumes of parenteral or enteral medicines to a patient. Intravenous, subcutaneous, intramuscular, and enteral administration of large volumes are provided by the disclosure herein. More specifically, one of more embodiments of the disclosure allows medications currently limited to the clinic setting to be administered at home by patients or lay caregivers, without the need for highly trained healthcare professionals or clinic visits. As a result, one of more embodiments of the disclosure is ideally suited for home administration of large volume biologics, such as monoclonal antibodies.

Embodiments described herein provide drug delivery apparatus, system or methods with a configurable plurality of medication reservoirs to administer a variety of medication regimens, including multi-drug regimens, as are common in oncology. Regimens may be administered over time in a sequential, parallel, time-delayed, or contingent manner. In one or more embodiments, the drug delivery system is provisioned with an interface to an electronic health record system and one or more medication orders or order sets, allowing administration of a multi-drug regimens and contingent medication administration based on laboratory values or physiologic monitoring. One of more embodiments of the disclosure provides home administration of more complex medication regimens that exceed the capability of existing prior art devices.

In one or more embodiments, tubing sets are provided with restricted flow rates corresponding to one or more clinical trial conditions or dosing regimens for an approved medication. One of more embodiments of the disclosure also provides both pre-approval clinical trials and commercialized medicines to be administered with the same device, apparatus or system, greatly reducing cost, time to market, and device, apparatus or system complexity.

In one or more embodiments, reservoirs may be individually designed for short- or long-term drug stability, based on the medication regimen being administered with the device, apparatus or system. Reservoirs may be filled at point of use in the home by a patient or caregiver, by a dispensing pharmacy, or by a pharmaceutical manufacturer. Optionally, the drug delivery system may be configured with intravenous flush solutions before and after administration in some embodiments.

In one or more embodiments, the drug delivery system is provisioned with a controller, algorithm, and sensors coupled to the controller to detect a patient's potentially life-threatening systemic infusion reaction. Further, embodiments of the drug delivery apparatus, system and method can administer a countervailing emergency medication in response to a systemic infusion reaction autonomously or at the direction of a remote clinician monitor, permitting home administration of medications that would otherwise be confined to in-clinic administration due to monitoring requirements and safety considerations. Moreover, in one or more embodiments, the drug delivery system is configured to deliver prophylactic medications before and after a medication with propensity for causing infusion reactions.

In one or more embodiments, the drug delivery system is provided with an input/output interface to a clinical trial data management system. In some embodiments, the data management system contains permanent storage for data collected during the clinical trial from one or more drug delivery systems herein. In some embodiments, data within the permanent data storage is used to support a regulatory submission for drug approval. In some embodiments, one or more drug delivery apparatus or systems is associated with one or more investigational therapeutic medications and/or clinical trial administration conditions for a specific patient.

### Patient Interface

Selection of the physiologic administration route dictates the patient interface used to deliver medication to the patient. While the most common physiologic routes are shown in FIGS. 1A through 1D, many other configurations of a patient interface will be apparent those skilled in the art, and descriptions herein are for illustrative purposes only, and shall not be construed as limiting the present disclosure.

Referring to FIGS. 1A and 1i, for patients receiving medication via a peripheral intravenous catheter (PIV) 115 or central venous access device (CVAD) 107 and 103, the patient interface 104 is provided by means of a Luer-Lok® or luer taper connection familiar to those skilled in the art. For patients receiving medication via an implanted venous port 127 and catheter 128, the patient interface 125 is provided by means of percutaneous access to the needle entry septum 129 with a specialized steel needle, such as a Huber needle 124.

Referring to FIG. 1C, for patients receiving medication via the subcutaneous route the patient interface comprises a subcutaneous (SC) needle assembly 140 and 158 placing needles at 900 142 or 450 160 to the injection site, thereby accessing to the SC tissue 148 and 175, through hollow-bore needle points 143 and 170. For intramuscular (IM) administration with embodiments of the apparatus or system, the patient interface comprises an IM needle assembly 151, wherein a hollow-bore needle 155 is placed into the patient's muscle tissue 149 through open needle point 156. The material of needles 142, 155, and 160 are siliconized rigid medical grade stainless steel common in the art. Medications are delivered to the patient via integral tubing sets 145, 154, and 172.

Referring to FIG. 1D, for patients receiving medication via the SC or IM routes the patient interface may alternately comprise a flexible soft cannula placed by a removable, rigid inserter needle. A needle assembly 181 is inserted against the patient skin 182 by a patient or caregiver 180, optionally using one or more insertion affordances 186. Upon placement against the patient skin 182, a first portion of needle assembly 181 is removed by the user 188, retaining a portion 191 in the skin comprising the soft, flexible cannula 192 with open tip 194. The first removed portion of the needle assembly 189 comprises the steel inserter cannula 190 and the insertion and removal affordances 189. The retained portion of the needle assembly 191 includes a tubing set 195 for medication administration to the patient's SC tissue 193. IM administration is also provided simply by increasing the length of the flexible cannula 183 and inserter needle 184 to place the open end of the flexible cannula 194 into the patient's muscular tissue 195. The material of the inserter needle 190 is rigid siliconized medical grade stainless steel, and the material of the flexible administration cannula 184 may be any biocompatible polymer, such as PFTE.

### Drug Delivery System Components

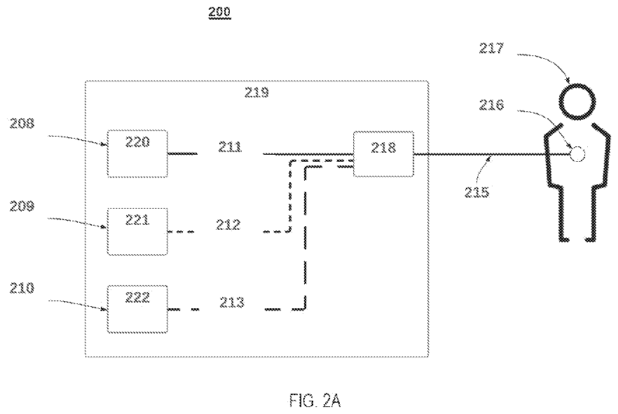

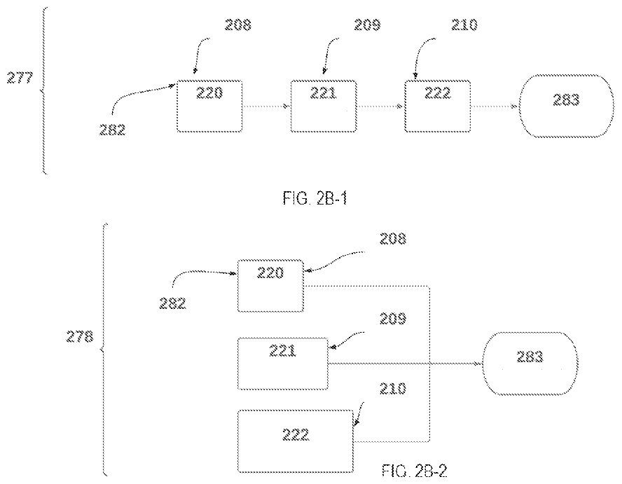

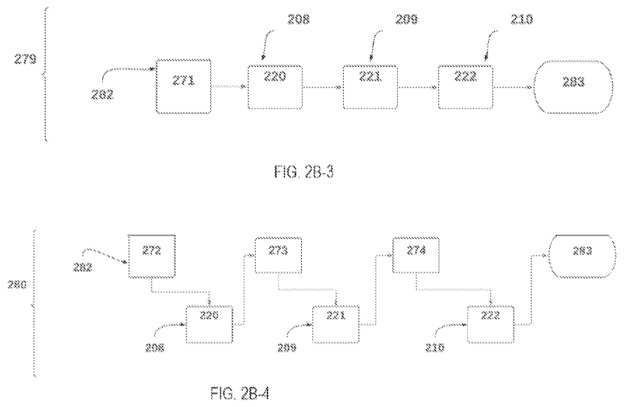

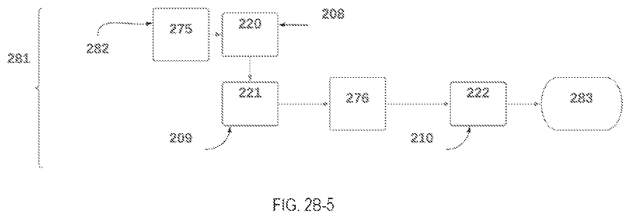

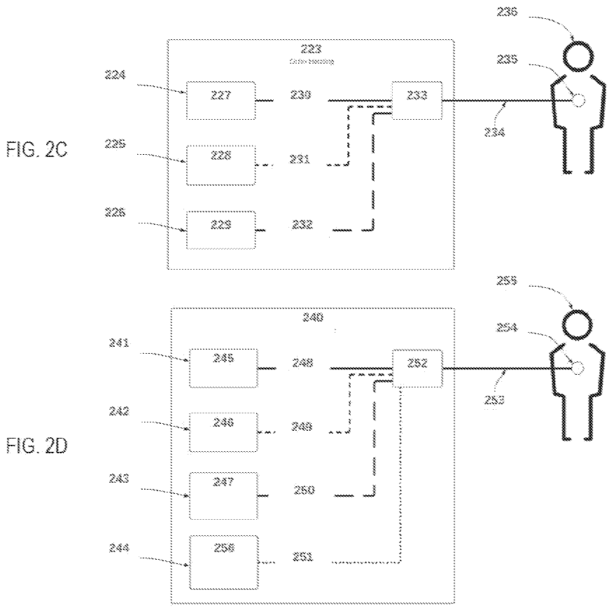

FIG. 2A illustrates variations of an exemplary drug delivery apparatus or system comprising an outer housing 219, a plurality of reservoirs 208, 209, and 210 for one or more therapeutic medication(s) 220, 221, and 222, fluidically connected 211, 212, and 213 to a fluid pump 218, by which the reservoirs may be emptied by the fluid pump 218 to administer the medication to the patient 217 by way of a tubing set 215 and patient interface 216. Although three reservoirs 208, 209, and 210 are described herein, many configurations of reservoirs are apparent based on the desired medication regimen, and are presented for illustrative purposes only, without limiting the present disclosure. As the exemplary embodiments make clear, any number of medications can be administered by the present system as desired.

In some embodiments, the outer housing 219 substantially encloses one or more reservoirs 208, 209, 210 and fluidic communication 211, 212, 213 between the reservoirs and fluid pump 218. In some embodiments, the outer housing 219 substantially encloses the fluid pump 218 and fluidic communication 211, 212, 213 between the reservoirs and fluid pump 218, and partially encloses one or more reservoirs 208, 209, 210.

In some embodiments, the outer housing is a rigid enclosure. In some embodiments, the outer housing is substantially flexible to conform to a patient's body or pocket. In some embodiments, the outer housing is configured with a single contoured side oriented towards and situated to conform to the patient's body. In some embodiments, the rigid plastic material, such as polypropylene, polycarbonate, acrylonitrile butadiene styrene, polyamide, or polystyrene. In some embodiments, the outer housing is over-molded on the side closest to the patient's body with a soft, compliant material, such as thermoplastic elastomer or thermoplastic polyurethane. In some embodiments, the outer housing is provided with a soft, compliant gel material on the side closest to the patient's body. In some embodiments, the outer housing is configured with a clip to allow attachment to a patient's clothing, pocket, or belt.

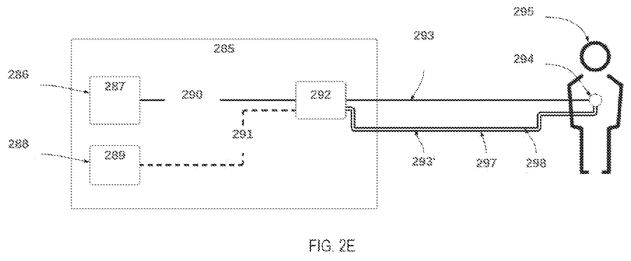

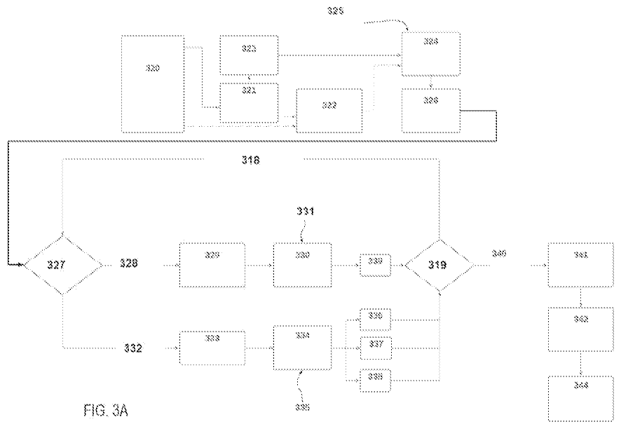

Referring to FIG. 2B, embodiments of the present drug delivery apparatus or system provide sequential, concurrent, time-delayed, and contingent administration of a variety of medications in a time sequence with a beginning 282 and end 283. During the time sequence, a plurality of medications 220, 221, 222 may be delivered in a prescribed sequential order 277 (as shown in FIG. 2B-1), in a concurrent manner 278 (as shown in FIG. 2B-2), in a prescribed sequential order 279 (as shown in FIG. 2B-3) in beginning after a prescribed time-delay 271, or in a in a prescribed sequence 280 (as shown in FIG. 2B-4) separated by one or more equally or unequally spaced time-delays 272, 273, and 274. Alternatively, during the time sequence, a plurality of medications 220, 221, 222 may be delivered in a prescribed sequence 281 (as shown in FIG. 2B-5), wherein certain medications are administered concurrently 220 and 221 after an optional time delay 275, after which other medications 222 are administered after a prescribed time-delay 276. The foregoing examples are for illustrative purposes and shall not be construed as limiting the number of medications or configurations that will be apparent to those skilled in the art.

FIG. 9 illustrates variations of an exemplary drug delivery apparatus or system comprising an outer housing 801, a plurality of reservoirs 807′, 808′ for one or more therapeutic medication(s) 807, 808 fluidically connected 809, 810 to a fluid pump 811, by which the reservoirs 807′, 808′ may be emptied by the fluid pump 811 to administer the medication 807, 808 to the patient 814 by way of a tubing set 812 and patient interface 813. Although the plurality of reservoirs 807′, 808′ shows only two reservoirs, this is for illustration only, and the apparatus and systems described herein are not limited to a particular number of reservoirs. In one or more embodiments there can be any suitable number of reservoirs. The drug delivery system is also provided with a controller 803 that communicates with components of the apparatus via either a wired or wireless connection. In one or more embodiments, the controller according to one or more embodiments comprises a processor 804, a memory coupled to the processor 805, input/output devices 806 coupled to the processor 805 and support circuits to provide communication between the different components of the system, namely the components of the system described herein. In one or more embodiments, processes to operate the system are stored in the memory 805 as a software routine that, when executed by the processor, causes the system to perform methods described in the present disclosure. In one or more embodiments, the processes to operate the system are performed in hardware. In one or more embodiments, the software routine to operate the system may also be stored and/or executed by a second processor that is remotely located from the hardware being controlled by the processor. In some embodiments, the second processor comprises a cloud computing service or server. In some embodiments, the second processor comprises a remote patient monitoring system used by a healthcare provider. In some embodiments, the second processor comprises an electronic health record (EHR) system interface. In some embodiments, the second processor comprises a clinical trial data management system interface. In some embodiments, the second processor comprises a smartphone, smart tablet, smart television set, or voice activated assistant.

In one or more embodiments, one or more input/output devices 806 comprises a light source that may be illuminated upon receiving instructions or a signal from the controller 803. In one or more embodiments, the light source is coupled to an optical conductor in the tubing set 812. In one or more embodiments, one or more input/output devices 806 comprises a power source electrically coupled to a conductor within the tubing set 812.

In one or more embodiments, controller 803 may be also coupled to the fluid pump 811 to sense and/or control fluid flow therein. In one or more embodiments, controller 803 may be also coupled to one or more fluidic connections 809, 810 sense and/or control fluid flow therein.

In one or more embodiments, controller 803 may be also coupled to one or more sensors and reservoirs 807′, 808′ containing medication 807, 808. In one or more embodiments, the outer housing 801, reservoirs 807′, 808′, and/or tubing set 812 may be configured with sensors also coupled to the controller 803. In one or more embodiments, controller 803 may be also coupled to one or more sensors 815 on the patient 814.

### Therapeutic & Other Medications

Various medications may be delivered by the present disclosure, including therapeutic medications, prophylactic pre-medications, prophylactic post-medications, emergency medications, and flushing solutions. Thus, “therapeutic medication” is used as a term of convenience herein to distinguish medications used to treat a disease (e.g., an oncology agent) from other ancillary medications delivered by the system while administering a therapeutic medication (e.g., a premedication or saline flush).

In some embodiments, a therapeutic medication is for treating one or more diseases selected from the group of cardiovascular, gastrointestinal, autoimmune, immunologic, hematologic, oncology, endocrinology, and respiratory disease. In some embodiments, a therapeutic medication is a coformulation of one or more medications for treating one or more of the aforementioned diseases. In some embodiments, multiple therapeutic medications are provided as part of a combination therapy.

In some embodiments, one or more therapeutic medications is a small molecule drug, therapeutic protein, cytokine, hormone, blood product, biologic, monoclonal antibody, antibody-drug conjugate, bispecific antibody, fusion protein, chimeric antigen receptor T cell therapy, cell or gene therapy, oncolytic virus, or immunotherapy.

In some embodiments, one or more therapeutic medications is an immuno-oncology or bio-oncology medication. In some embodiments, one or more therapeutic medications is selected from the group of several proposed targets, such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

In some embodiments, one or more therapeutic medications is selected from the group of proposed mechanisms of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

In some embodiments, one or more therapeutic medications is selected from one of ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

In some embodiments, one or more therapeutic medications is a part of a multi-medication treatment regimen. In some embodiments, one or more therapeutic medications is a part of a multi-medication treatment regimen selected from the group of AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

In some embodiments, one or more therapeutic medications is used for adjuvant chemotherapy. In some embodiments, the chemotherapeutic compound is used for neoadjuvant chemotherapy. In some embodiments, the chemotherapeutic compound is an alkylating agent, plant alkaloid, antitumor antibiotic, antimetabolite, or topoisomerase inhibitor, enzyme, retinoid, or corticosteroid. In some embodiments, the chemotherapeutic compound is selected from the group of 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

In some embodiments, one or more therapeutic medications is classified as a hazardous medication according to the Centers for Disease Control's “NIOSH List of Hazardous Drugs In Healthcare Settings” or as defined by US Pharmacopeia General Chapter <800>“Hazardous Drugs—Handling in Healthcare Settings.”

When administering certain therapeutic medications, prophylactic medicines may be administered to a patient before (pre-medication) or after a therapeutic medication (post-medication) to avoid systemic infusion reactions or ease discomfort from a therapeutic medication's side effects. The pre-medication and post-medications may also comprise part of a medication regimen or medication order set, described elsewhere herein.

FIG. 2C illustrates an exemplary drug delivery apparatus or system configured to administer certain prophylactic medicines in addition to one or more therapeutic medications also contained within the system. In one or more embodiments, the drug delivery apparatus or system 223 contains a plurality of reservoirs for medication 224, 225, and 226. In some embodiments, reservoir 224 contains one or more prophylactic pre-medications 227 administered before the therapeutic medication(s) 228. In some embodiments, administration of the therapeutic medication 228 can take place only after complete administration of required pre-medication 224. In some embodiments, reservoir 226 contains one or more prophylactic post-medications 227 administered after the therapeutic medication(s) 228.

In one or more embodiments, one or more reservoirs 224 or 226 contains one or more medications selected from the group of 0.9% normal saline, 0.45% normal saline, 5% dextrose in water, 5% dextrose in 0.45% normal saline, Lactated Ringer's solution, albumin, and crystalloid fluids containing added electrolytes, such as potassium.

In one or more embodiments, one or more reservoirs 224 or 226 contains one or more medications selected from the group of analgesics, antipyretics, corticosteroids, antihistamines, antiemetics, antibiotics, anticoagulants, fibrinolytics, or antithrombolytics. In one or more embodiments, one or more reservoirs 224 or 226 contains one of diphenhydramine, acetaminophen, ondansetron, or famotidine.

In one or more embodiments, one or more reservoirs 224 or 226 are configured to reconstitute a lyophilized pre-medication or post-medication contained in a dual-chamber syringe featuring a bypass chamber. In one or more embodiments, one or more reservoirs 224 or 226 are configured to reconstitute a lyophilized pre-medication or post-medication in an anticipatory fashion to allow more timely administration.

When administering medications intravenously, it is necessary to flush the IV catheter system before and after medication administration. Flushing refers to the process of instilling a fluid volume after therapeutic medication delivery through the entire IV system to ensure all medication within the IV system is fully administered to the patient and to prevent clotting of the catheter system. In one or more embodiments, the drug delivery apparatus or system may also be configured to deliver of a therapeutic medication in conjunction with catheter flushing protocols.

Referring to FIG. 2D, in one or more embodiments, the drug delivery apparatus or system is provided with flush reservoirs 241, 243, 244 and a reservoir for therapeutic medication 242. The delivery apparatus or system is configured to deliver one or more catheter flushing solutions before 245 and/or after 247, 256 administration of one or more therapeutic medications 246. In one or more embodiments, the delivery apparatus or system administers an 0.9% Normal Saline from a pre-administration flush reservoir 241 followed by one or more therapeutic medications 246 in a reservoir 242, followed by an 0.9% Normal Saline Flush in a first post-administration flush reservoir 243, followed by Heparin Lock Flush solution in a second post-administration flush reservoir 244. Flushing need not be limited to the beginning and end of an administration process; when multiple medications are administered, flush reservoirs may be interposed between therapeutic medication administrations if desired.

In some embodiments, one flushing solution is 0.9% Normal Saline. In some embodiments, one flushing solution is recombinant tissue plasminogen activator (r-TPA). In some embodiments, one flushing solution is one or more medications selected from the group of 0.9% Normal Saline, Heparin Lock Flush solution, 100 U/mL Heparin Lock Flush Solution, and 5000 U/mL Heparin Lock Flush Solution. In some embodiments, one flushing solution is an antimicrobial. In some embodiments, one flushing solution is an antimicrobial combined with an anticoagulant.

### Tubing Set

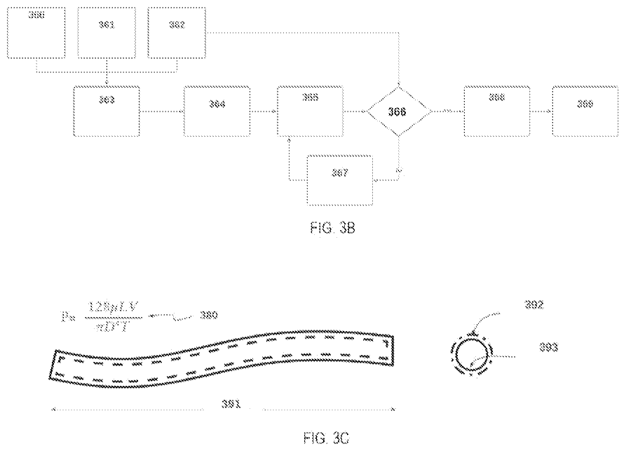

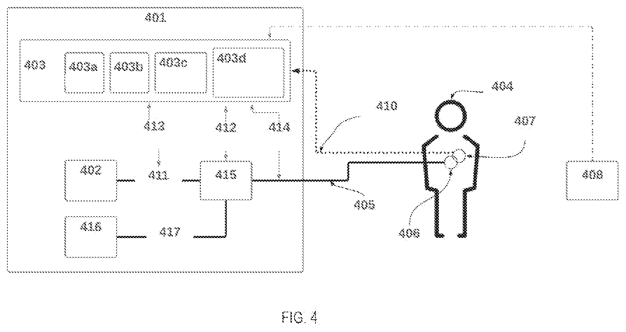

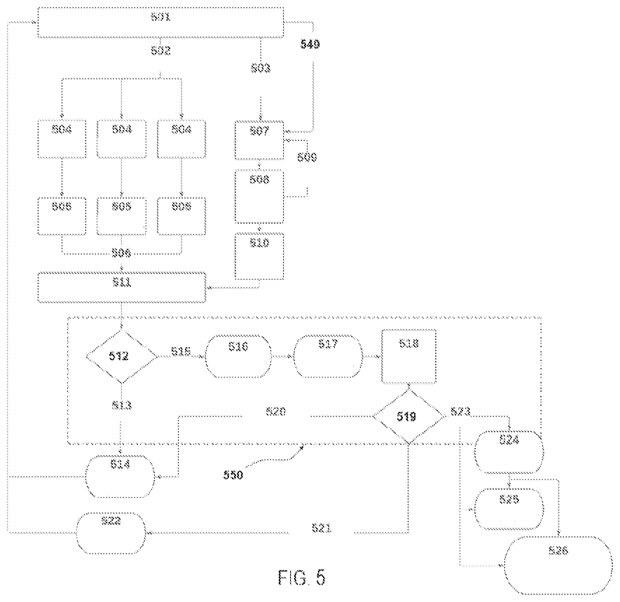

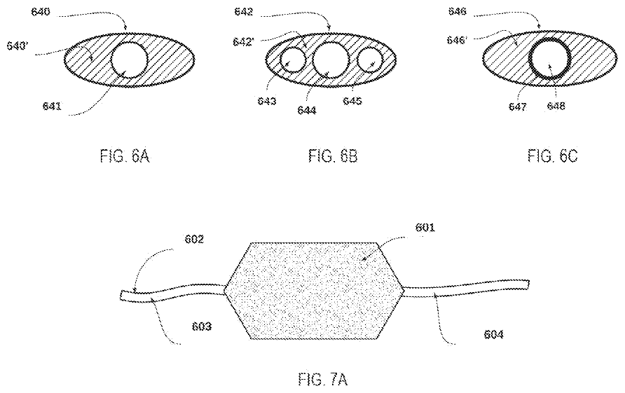

FIGS. 6A-C illustrate variations of an exemplary tubing sets for use with the present disclosure. In one embodiment, a tubing set 640 is provided with cross-sectional tubing profile 640′ and at least one inner medication lumen 641. During use of the drug delivery system, inner medication lumen 641 is in fluidic communication with a fluid pump and a patient interface described elsewhere herein to deliver medications within the system to the patient.

It may be desirable to isolate one or more inner medication lumens 648 from potential contaminant leachable or extractable compounds from the tubing set material, thereby improving compatibility with the medication delivered therein. Accordingly, in some embodiments, barrier coating 647 may be interposed between an inner medication lumen 648 and tubing set material 646′. In one embodiment, the barrier coating comprises a PTFE fluoropolymer material. In another embodiment, the barrier coating is co-extruded as the tubing set is manufactured. In another embodiment, the interior medication-contacting surface of one or medication lumens are provided with a hydrophobic coating.

It may be desirable to offer multiple flowrates in the present drug delivery system without switching tubing sets. Accordingly, in one embodiment, a tubing set 642 is provided with cross-sectional tubing profile 642′ and two or more medication lumens 643, 644, 645. The medication lumens may have different or similar diameters, thereby allowing administration of medications at flow rates in a variety of configurations. By way of example, the same medication administered through a first lumen 644 would flow more quickly than if administered through a second lumen 643 in the tubing set design exemplified in FIG. 6C. In an alternative embodiment, medication delivery may be accelerated by switching flow from a smaller to a larger lumen (e.g., from 643 to 645). In an alternative embodiment, medication delivery may be decelerated by switching flow from a larger to a smaller lumen (e.g., from 645 to 643). In an alternative embodiment, one or more medication lumens may be engaged in parallel fashion (e.g., using 643 and 645, or 644 and 643) to provide faster administration of a single medication. In an alternative embodiment, one or more medication lumens may each deliver a different medication concurrently. In an alternative embodiment, one or more medication lumens remains unused by the system until desired, as in the case of emergency medication administration as described herein.

Elements of the tubing sets described herein may take various shapes and forms. In one or more embodiments, cross-sectional tubing profiles may take a substantially circular, elliptical, rectangular, or polygonal shape. The flexible portion of the tubing set may be fashioned from one or more of silicone, PVC, PVC without DEHP, EVA, HDPE, LDPE, TPU, PTFE, a fluoropolymer, or other suitable flexible material. In one or more embodiments, tubing sets are extruded but may be formed by other means that provide sufficient dimensional and tolerance control on the inner medication lumens as described herein. In one or more an embodiments, the tubing material is chosen to be a material selected for low leachable and extractable compounds that may contaminate a medication, and that exhibits high biocompatibility with biologic medications.

Optionally, the flexible portion of the tubing set may comprise segments of one or more flexible materials, providing different degrees of flexibility at different sections along the length. For instance, a more rigid material may be provided near the connections to the fluid pump for strain relief and anti-kinking, while a more flexible material may be selected near the patient interface for comfort against a patient's skin. The exterior of the tubing set may be provided with a PFTE fluoropolymer or other permanently lubricious coating to prevent dragging or snagging of the tubing set on a patient's skin or clothing.

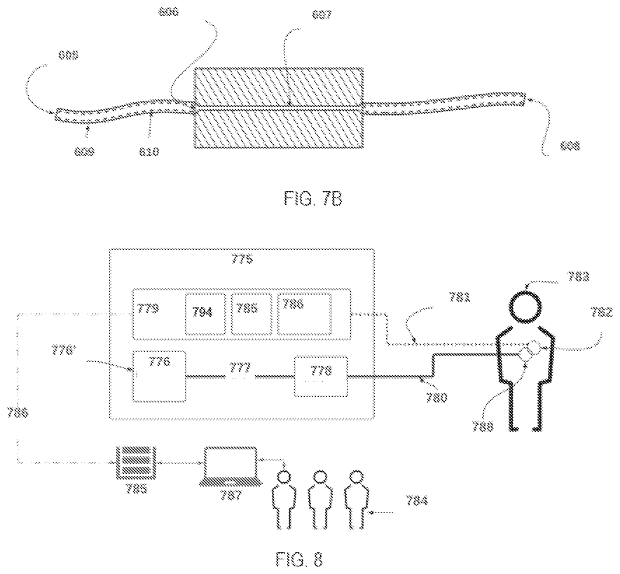

In one or more embodiments, and referring to FIGS. 7A-B, one or more tubing sets is provided with an inline filter 601 to remove undesirable or immunogenic particulate matter 602 prior from the inflow medication 603 prior to patient administration at the outflow side of the filter 604. The inline filter material is ideally selected to be low-sorbing, low protein binding, and compatible with the medication(s) therein. Optionally, the inline filter may comprise a multi-layer filter membrane, with each membrane layer featuring a different filter pore size.

In one or more embodiments, one or more tubing sets are provided with an engineered flow restriction 607 to provide an inflow medication 605 at a first rate, and outflow medication 608 at a second rate substantially less than the first rate. When used with biologic or shear-sensitive medications, the smoothed inlet 606 and engineered flow restriction 607 is in one or more embodiments designed to prevent protein damage or shearing.

### Fluid Pump

A variety of fluid pumps may be used in the disclosure herein, based on the configuration of reservoirs, viscosity of medications, and number of medications. In some embodiments, a single fluid pump is provided. In some embodiments, multiple fluid pumps are provided. In some embodiments, the fluid pump is configured to start, pause, or stop on demand. In some embodiments, the fluidic pump is configured with a transmission mechanism to provide selective engagement and disengagement of selected medication reservoirs. In some embodiments, the mechanical drive is coupled to a gear mechanism to reduce the form factor of the apparatus or system. In some embodiments, the gear mechanism comprises mating bevel gears. In some embodiments, the fluidic pump is prevented from operation if one or more medications is insufficiently viscous. In some embodiments, the fluidic pump is provided with a sensor to determine the temperature of a fluid at the fluid pump inlet.

Fluid pumps may be powered by, for example, a flat coil spring, wound helical spring, strip spring, pressurized gas, or an electrical motor. In some embodiments, a rotary power source may be coupled to one or more reservoirs through a worm screw and worm gear. In some embodiments, the worm screw and worm gear is used to hold a reservoir in a given position while other reservoirs are driven by the system. In another alternative embodiment, the fluid pump be driven by a power unit with rate control assembly, such as disclosed in U.S. Pat. No. 10,252,005. In another alternative, the fluid pump may be driven by a chemical engine, such as disclosed in U.S. Pat. No. 9,795,740. In another embodiment, the fluid pump may be drive by a power unit with progressive engagement mechanism, such as disclosed in U.S. Pat. No. 10,357,612. In another embodiment, the fluid pump may be driven by a rotary drive, such as disclosed in U.S. Pat. Nos. 8,617,109, 8,876,766, 9,022,982, 9,095,657, 9,132, 236, 9,446,201, 9,468,722, 9,737,668, 10,255,827, 10,307,543, 10,456,521, 10,507,289, 10,525,213, 10,632,248, 10,874,804, 10,881,811 and 11,065,387, the entire contents of each of these patent documents incorporated by reference in their entirety.

In an alternative embodiment, the fluid pump is a one-time use disposable design. In an alternative embodiment, the fluid pump is a reusable design for multiple medication administrations. In an alternative embodiment, the fluid pump is a reusable design designed to administer a single cycle of a medication regimen.

In one or more embodiments, one or more fluidic connections are designed to minimize internal volume that is not administered to the patient, thereby reducing medication waste and the need for medication overfill. Accordingly, in one or more embodiments, fluidic connections between one or more reservoirs and the fluid pump may comprise a manifold. In an alternative embodiment, each fluidic connection between one or more reservoirs and the fluid pump may have proportionally different relative to each other, permitting independent flow rate control of one or more medications beyond that provided by one or more tubing sets provided with the drug delivery system.

### Fluid Pump+Tubing Set Integration

In an embodiment, the fluid pump is sufficiently well-powered to deliver a full range of volumes, viscosities, and rates independently of the inner diameters of a tubing set, thereby allowing the same fluid pump design to be used for a variety of medications. This has the advantage of mass-producing fluid pumps and gaining efficiencies of scale. This approach allows design of a drug delivery apparatus or system without knowing medication formulation characteristics a priori. This is particularly important in clinical trials, where medication formulation characteristics are still in development, and dosing regimens are not yet finalized.

It is apparent that tubing sets in the present disclosure are used to control administration parameters for a therapeutic medication and accommodate flow characteristics of specific drug formulations without the need for complex or precise mechanical or electromechanical pumps. This is particularly important for biologic drug products or extended-release formulations displaying non-Newtonian shear-thinning and shear-thickening behaviors where modeling techniques are of limited usefulness.

FIG. 3B depicts an embodiment of a process to design tubing sets for use in a clinical trial in accordance with the disclosure herein. Formulation characteristics 360, pharmacokinetic modeling parameters 361, and desired clinical trial conditions 362 are inputs to initial numeric modeling 363 using either Hagen-Pouiselle's equation 380 (FIG. 3C) or other modeling methods, such as computational fluid dynamics. Modeling 363 provides initial design and component selection 364, comprising minimally first estimated nominal tubing lengths 391, tubing nominal internal diameters 392, and corresponding tolerances 393 on the nominal internal diameters 392 (FIG. 3C).

Tubing may be manufactured based on initial design and component selection 364. However, for non-Newtonian fluids, initial numeric modeling 363 may be substantially different than predicted, and adjustments to tubing internal diameters 392, and corresponding tolerances 393 on the internal diameters 392 may be required. The adjustments may require time-consuming or costly changes to extrusion dies or other equipment, and multiple testing and adjustment cycles may be required.

Regardless, the flow rate provided by the initially selected components 364 are physically tested 365 with the drug formulation of interest and compared to the desired clinical trial conditions 324, 330, 334, and 342. Physical testing 365 may optionally include characterization of any damage to the drug product caused by the tubing set or flow rates, including protein damage or shearing effects which may render protein-based medications inactive or harmfully immunogenic to humans. Physical testing 365 may optionally be conducted at temperatures representative of the administration setting for the final medication in clinical practice, which is especially relevant for medications that exhibit a nonlinear viscosity-temperature-concentration relationship, such as biologics.

As many medications display non-Newtonian shear-thinning and shear-thickening behaviors, empirical results may also differ from theoretical calculations, in which case components are iteratively redesigned 367. Individual tubing sets corresponding to a specific flow rate for a specific medication are individually analyzed, refining either tubing lengths 391 or tubing diameters 392, or specifying precision tolerances 393 on the diameters 392. Once precisely designed, a plurality of tubing sets is manufactured 368 for use with the overall drug delivery system to execute a given clinical study design 369 as previously specified.

### Medication Reservoirs

Referring to FIG. 2A in one or more embodiments, medication reservoirs 208, 209, and 210 are designed for short-term duration contact with therapeutic medications 220, 221, and 222, minimizing the technical burden and risk associated with long-term stability or container closure testing. In an alternative embodiment, the reservoirs 208, 209, and 210 are each selectively designed for short- or long-term drug contact based on the nature of the medicine 220, 221, and 222 therein.

Referring to FIG. 2C, in one or more embodiments, medication reservoirs 224 and 226 are long-term stability primary containers that are prefilled with medications 227 and 228, and reservoir 225 with a therapeutic medication 228 is designed for short-term stability and filled just prior to administration.

In one or more embodiments, one or more reservoirs is a glass or plastic syringe, or cartridge prefilled by the manufacturer. In one or more embodiments, the interior surface of one or more reservoirs contains controlled levels of a silicone lubricant. Optionally, the silicone lubricant may be crosslinked, as through radiation. In one or more embodiments, one or more reservoirs is a single syringe with a plurality of reservoirs, chambers, or compartments.

In an embodiment of the of the present disclosure, one or more reservoirs is a flexible nonelastic container. In one or more embodiments, the flexible nonelastic container is fully emptied through application of a compressive force. Optionally, the flexible nonelastic container may be contained in a rigid protective shell. In one or more embodiments, one or more reservoirs is a flexible elastomeric container. In one or more embodiments, one or more reservoirs is a flexible container with one or more segments, each containing a single medication.

In some embodiments, one or more reservoirs are manufactured from one or more materials selected from the group of borosilicate glass, cyclic olefin polymer, cyclic olefin copolymer, PVC, EVA, fluorinated ethylene propylene (FEP) resins or films, PTFE, a fluoropolymer, or other suitable material. In other embodiments, one or more reservoirs are manufactured from a low-sorbing material. In some embodiments, one or more interior reservoir surfaces in contact with medication has a hydrophilic coating or has been passivated to reduce protein sorbing or formation of protein aggregates.

In some embodiments, the reservoirs are filled by pharmacy before dispensing to a patient. In some embodiments, the reservoirs are filled by a patient or caregiver at home. In some embodiments, the reservoirs are prefilled and assembled into the drug delivery system prior to use by a patient. In one or more embodiments, one or more reservoirs is filled while contained in the drug delivery apparatus or system. In one or more embodiments, one or more reservoirs is filled outside the drug delivery apparatus or system, then installed into the drug delivery system as a secondary operation. In one or more embodiments, one or more reservoirs is filled by the patient, lay caregiver, or healthcare provider. In an alternative embodiment, one or more medication vials are provided with a vial transfer apparatus or system for filling a reservoir. In an alternative embodiment, the reservoir is pre-attached to a transfer apparatus or system to effectuate filling with a minimum of use steps and corresponding risk of aseptic breach. In an alternative embodiment, the reservoir is filled from a vial using pressure applied by a compressed gas. In an alternative embodiment, the reservoir is filled from a vial using pressure applied by an electromechanical pump assembly.

In one or more embodiments, the drug delivery system is equipped with one or more features to prevent unauthorized access to, or diversion of, one or more reservoirs containing a controlled substance after filling. The features may include a tamper-evident seal on the exterior of the drug delivery apparatus or system or internal sensors to detect unauthorized access to the drug delivery system and components within it, including medication reservoirs.

In some embodiments, one or more reservoirs is provided with a sensor to determine the temperature of a fluid therein. In some embodiments, the sensor is located on the exterior of the reservoir. In some embodiments, the sensor is a temperature probe making direct contact with the medication through the reservoir wall.

### Infusion Reaction Detection

Used herein as a term of convenience, infusion reactions include standard infusion reactions (SIRs), cytokine-release reactions, or IgE-mediated allergic reactions. As new categories of biologics with novel modes of action are developed and commercialized, additional types of patient infusion reactions may also become apparent beyond those listed herein. Thus, the foregoing infusion reactions cited herein are provided by way of example and shall not be construed as limiting the scope of disclosure of the disclosure herein.

Certain medications are associated with overall higher incidence of infusion reactions. For these medications, specific pre- and post-medications are administered to reduce incidence of infusion reactions or negative patient impacts should they occur. Administration of pre- and post-medications is provided by the present disclosure as illustrated in FIG. 2C and described elsewhere herein.

However, even when prophylaxis is administered, infusion reactions can occur. Infusion reactions are clinically distinct from injection site reactions, which cause localized discomfort and are neither emergent nor life threatening to the patient. Onset of infusion reactions is sudden, systemic, and life-threatening; treatment requires unexpected and immediate administration of counteracting emergency medications. Due to rapid onset, healthcare providers monitor patients routinely in the clinic setting and intervene immediately.

Due to the serious nature of infusion reactions, it is highly desirable to anticipate potential infusion reactions at onset, especially in settings outside the clinic, which is also provided by alternative embodiments of the drug delivery apparatus or system herein. FIG. 4 illustrates an exemplary drug delivery apparatus or system configured to include sensors to detect potential infusion reactions, a controller and algorithms, features to interrupt medication flow, and optional features for delivery of emergency medications in response to an infusion reaction.

Referring to FIG. 8, in one alternative embodiment, data from coupled sensors 815 is processed by an algorithm within the controller 803 configured to detect suspected infusion reaction and deliver appropriate therapeutic treatment automatically or through intervention by a healthcare provider. In some embodiments, the algorithm utilizes historical data from a single patient to determine whether an infusion reaction is occurring. In some embodiments, the algorithm utilizes historical data from one or more users of the drug delivery system to determine whether an infusion reaction is occurring. In some embodiments, the algorithm utilizes historical data from one or more prior clinical trials with the therapeutic medication being administered to determine whether an infusion reaction is occurring. In some embodiments, the aggregated historical data is analyzed by a machine learning program to improve accuracy or timeliness of infusion reaction identification. In some embodiments, the algorithm uses historical data aggregated from many patients in conjunction with machine learning to compute a probabilistic estimate of whether an infusion reaction is occurring in a present instance.

In one or more embodiments, the drug delivery apparatus or system is configured to halt administration of one or more therapeutic medications immediately if an infusion reaction is detected. In a first alternative embodiment, drug delivery may be halted by the controller 803 interrupting fluidic connection with the tubing set 812. In a second alternative embodiment, drug delivery system may be halted by the controller 803 stopping the fluid pump 811. However, both preceding alternative embodiments are disadvantageous, as no further medications may be administered, including a counteracting emergency medication. In a third alternative and an embodiment, administration of a therapeutic medication may be halted by the controller interrupting fluidic connection between the reservoir 807 and the fluid pump 811, while leaving the fluid pump 811 and tubing set 812 operable to provide administration of a counteracting emergency medication 808 contained in reservoir 808′.

Referring to FIG. 4, in an alternative embodiment, a drug delivery apparatus or system is provided with a reservoir 402 for containing a therapeutic medication, a reservoir 416 containing an emergency medication, and fluidic connections 411, 417 between the reservoirs and a fluid pump 415, a tubing set 405 fluidically connected between the fluid pump 415 and patient interface 406, one or more sensors 407, and one or more sources of patient data 408. Sensor data 410 is communicated to the controller 403 from the sensors 407. In one alternative embodiment, data from sensors 407 is processed by an algorithm within the controller 403 configured to detect suspected infusion reaction and deliver appropriate therapeutic treatment automatically or through intervention by a healthcare provider as described herein.

In one or more embodiments, the controller 403 according to one or more embodiments comprises a processor 403a, a memory coupled to the processor 403b, input/output devices 403c coupled to the processor 403a, and support circuits to provide communication between the different components of the system, namely the components of the system described herein. In one or more embodiments, processes to operate the system are stored in the memory 403b as a software routine that, when executed by the processor, causes the system to perform methods described in the present disclosure. In one or more embodiments, process to operate the system comprise an infusion reaction detection algorithm 403d based on one or more sensor data 410 from one or more patient sensors 407 or patient data 408. In one or more embodiments, patient data 408 comprises a self-report of symptoms by the patient 407. In one or more embodiments, patient data 408 is derived from a healthcare provider interaction with a patient 407. In one or more embodiments, the infusion reaction detection algorithm also is configured to respond to a detected infusion reaction in conjunction with the controller 403, whereby one or more emergency medications 416 may be administered, or whereby medication delivery may be halted to a patient 407 as described herein. In one or more embodiments, the processes to operate the system are performed in hardware. In one or more embodiments, the software routine to operate the system may also be stored and/or executed by a second processor that is remotely located from the hardware being controlled by the processor.

In one or more embodiments of the present disclosure, the drug delivery device is configured to halt administration of one or more therapeutic medications of interest immediately if an infusion reaction is detected. In a first alternative embodiment, the drug delivery system 401 may be provided with a fluid flow control 414 configured to interrupt fluidic communication between the fluid pump 415 and the tubing set 405. In a second alternative embodiment, the drug delivery system 401 may be provided with a fluid flow control 412 configured to interrupt the fluid pump 415 and cease all medication delivery to the patient 404.

However, both preceding alternative embodiments have the disadvantage that no further medications may be administered, including a counteracting emergency medication. Thus, in a third alternative and preferred embodiment, the drug delivery system 401 may be provided with a fluid flow control 413 configured to interrupt fluidic communication between the fluid pump 415 and a therapeutic medication reservoir 402, thereby preventing flow of a therapeutic medication 402 causing an infusion reaction, while leaving the fluid pump 415 and tubing set 405 configured to administer a counteracting emergency medication 416 to a patient 404.

FIG. 5 provides a schematic of an embodiment of the decision-making algorithm within the controller 403 and sensor(s) 407 referenced in FIG. 4, wherein diagnosis and treatment for infusion reactions are supported by the algorithm 403 as a form of decision support for a healthcare provider. The embodiment provides that during medication administration 501, the drug delivery system is configured to detect potential infusion reactions based on one or more of physiologic sensor data 502, in-person or remote observation of the patient's condition 503 by a healthcare provider, and patient self-report 527.

Physiologic data 502 for potential infusion reactions, may include by way of example but not limitation, heart rate, blood pressure, respiratory rate, blood oxygen saturation (SpO2), and temperature, which are collected by way of sensor(s) 407. The plurality of sensors sample the data 504, data is pre-processed 505 using the system's controller and algorithm 403 and the output is aggregated and consolidated 506, also by the controller and algorithm 403.

Sensor data may be supplemented with objective and subjective observation 507 of patients' conditions 503 from physical examination such as flushing, skin reactions, rigors, swelling, urticaria, angioedema, wheezing, stridor, cough, change in voice quality, or loss of consciousness. Sensor data may further be supplemented with data collected from patient interview or self-report 527, including by way of example, headache, shortness of breath, throat closing, diaphoresis, nausea, abdominal or back pain, itching, general anxiety, or self-reported sense of “impending doom.”

Observations of the patient 507 prompt in-person or remote patient interactions and/or patient interviews 508, which are aggregated and evaluated by the healthcare provider in a feedback loop 509 until the patient evaluation is satisfactorily completed, whereupon the healthcare provider uses their clinical judgement and heuristics to arrive at an overall patient assessment 510. Quantitative sensor data 506 and qualitative patient assessment 510 is thus consolidated 511 into an overall patient assessment, which is used to assess whether the patient is experiencing an ongoing infusion reaction 512 and determine the need for emergent treatment.

If an infusion reaction is not suspected 513, administration 501 may be continued at the ongoing administration rate 514. If an infusion reaction is suspected 515, the medication infusion is automatically paused or stopped 516, the patient's situation is immediately escalated, and relevant clinical staff are provided with the appropriate data 517. Upon evaluating the totality of data 517 and the patient 518, the healthcare provider determines whether it is safe to restart the infusion 519. If the healthcare provider determines that the patient is not having an infusion reaction (i.e. “a false alarm”) and it is safe to restart 520, the infusion may be continued at the same administration rate as previously tolerated 514.

If the healthcare provider determines that the patient is having a mild infusion reaction that can be remedied by slowing the infusion rate 521, the infusion may be continued at a reduced rate 522 pre-determined by the healthcare provider by administering medication using the smaller lumen of a multiple-lumen tubing as described elsewhere herein.

If the healthcare provider confirms the patient is experiencing an infusion reaction and determines it is unsafe to restart the infusion 523, they can opt to trigger an optionally provided feature within the drug delivery system to administer one or more emergency medications 524 and optionally call emergency medical services 525. In an alternative embodiment, the emergency medical services 525 are configured to provide a timelier response by virtue of geolocation data 526 provided by the drug delivery apparatus or system.

The treatment algorithm comprising 512, 513, 515, 516, 517, 518, 519, 523, and 524 is provided by way of example and not limitation. More generally, the present disclosure provides one of many alternative evaluation and treatment flows 550, which may be tailored based on the specific therapeutic medication, expected type and severity of infusion reaction, specifics in a prescribed medication order or order set, required counteracting medications, and other clinical considerations.

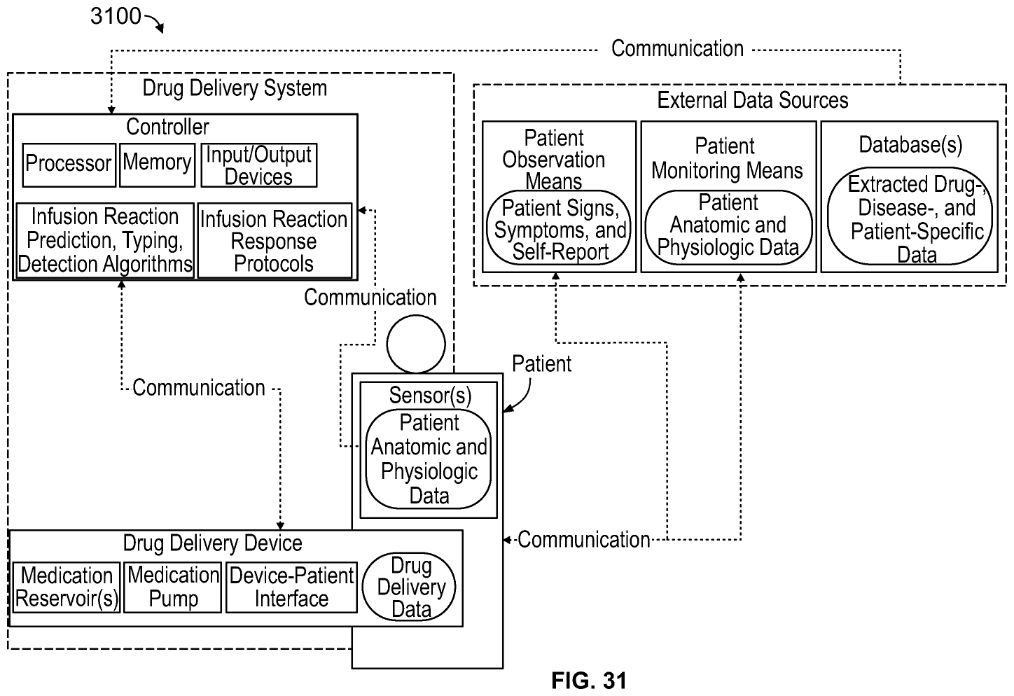

FIG. 31 schematically illustrates another possible embodiment of system 3100 for delivering medication to a patient that predicts, identifies, differentiates and/or responds to infusion reaction subtypes. More particularly, the invention relates to enabling a drug delivery system to safely and appropriately deliver medications that may be prone to eliciting infusion reactions without direct supervision from an HCP (e.g., in patients' homes), given the ability to understand the risk of their occurrence and respond in a closed-loop fashion. As with systems discussed above, there can be two main subcomponents, the drug delivery system operatively connected with a patient and an external data source in remote communication with the drug delivery system. The first drug delivery system subcomponent can include a drug delivery device directly in fluid communication with a patient through a medication pump via a device patient interface, where the pump is in fluid communication with one or more medication reservoirs. This first subcomponent can further include electronics, such as a controller having a processor, memory, input/output devices, infusion reaction prediction, typing and detection algorithms, as well as infusion reaction response protocols.

The controller is in two-way communication with the drug delivery device. Sensors attached to the patient are also part of the first subcomponent that monitor and collect patient anatomic, e.g., skin changes, and physiologic data, e.g., vital signs, serum or backpressure data that is transmitted to the controller. The controller is configured to process, store and transmit such data to the drug delivery device. These sensors can also transfer collected data to the second subcomponent, i.e., the external data source. Likewise, the patient can manually input into external data source self-reports, including observations, signs and symptoms being experience during operation of the first subcomponent. The external data source second subcomponent can also include accessible databases, for example, drug, disease and patient specific data, e.g., electronic health records, drug databases, clinical guideline databases. This second subcomponent can communicate directly with the controller section of the first subcomponent.

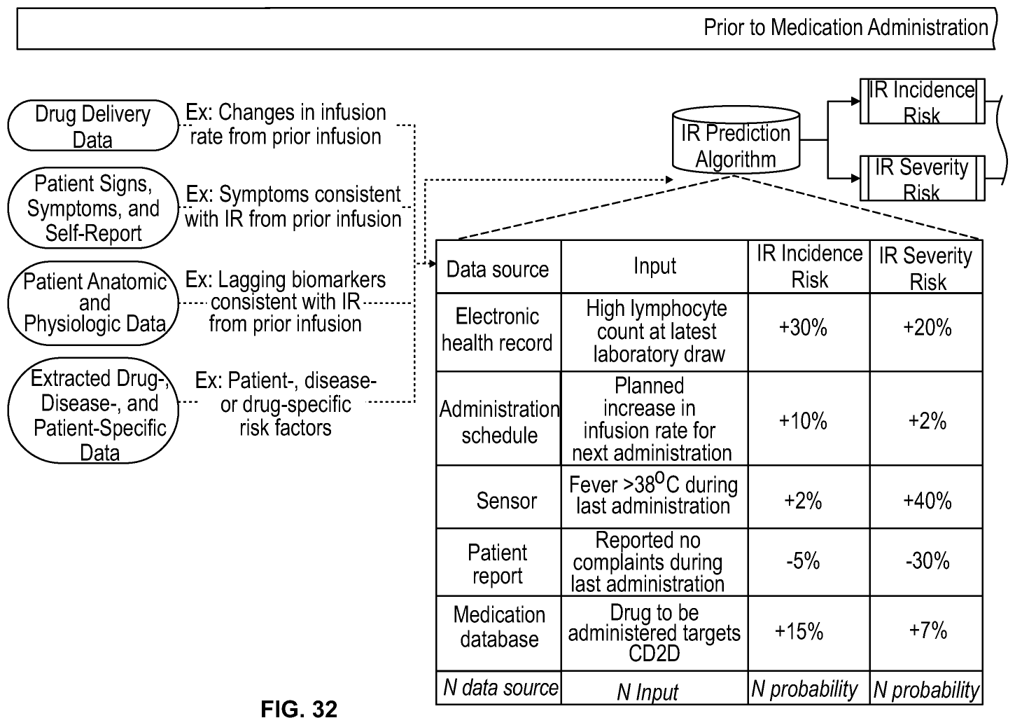

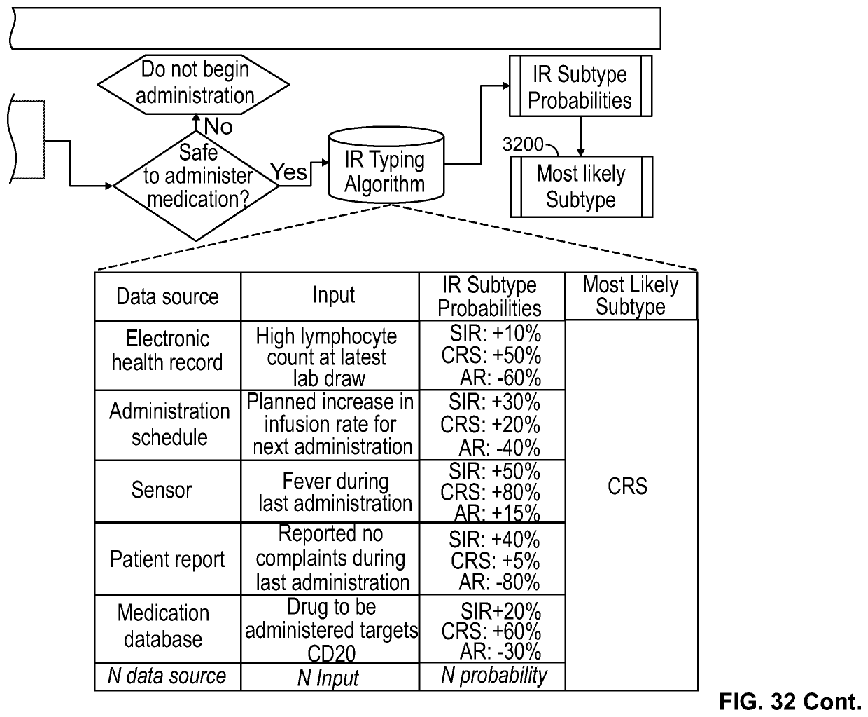

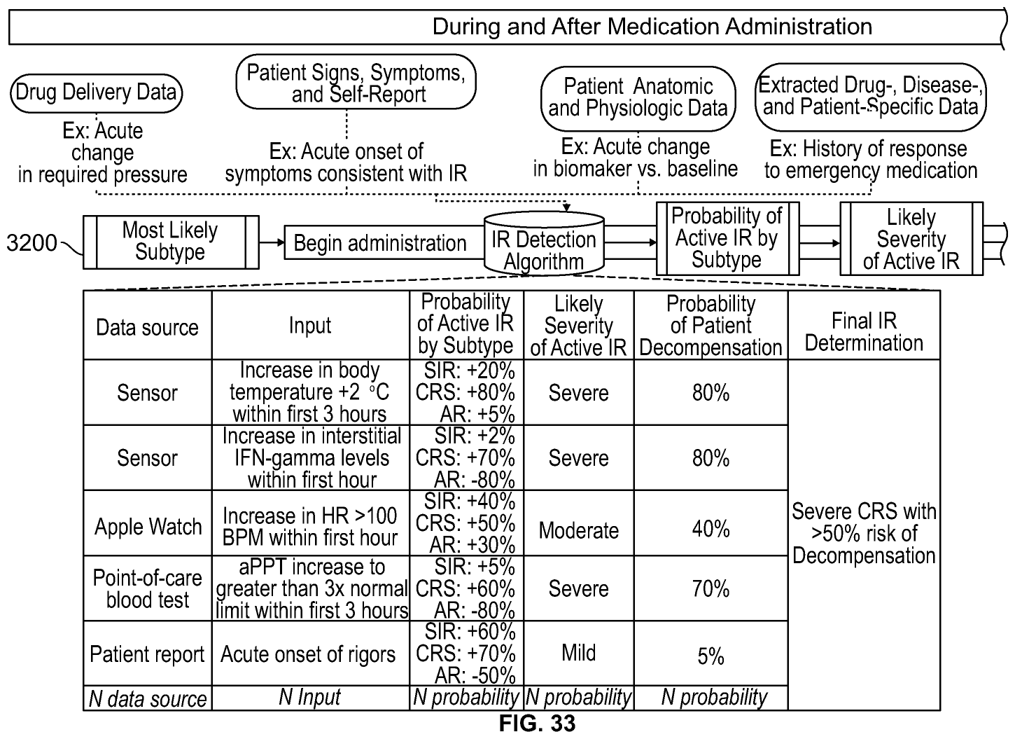

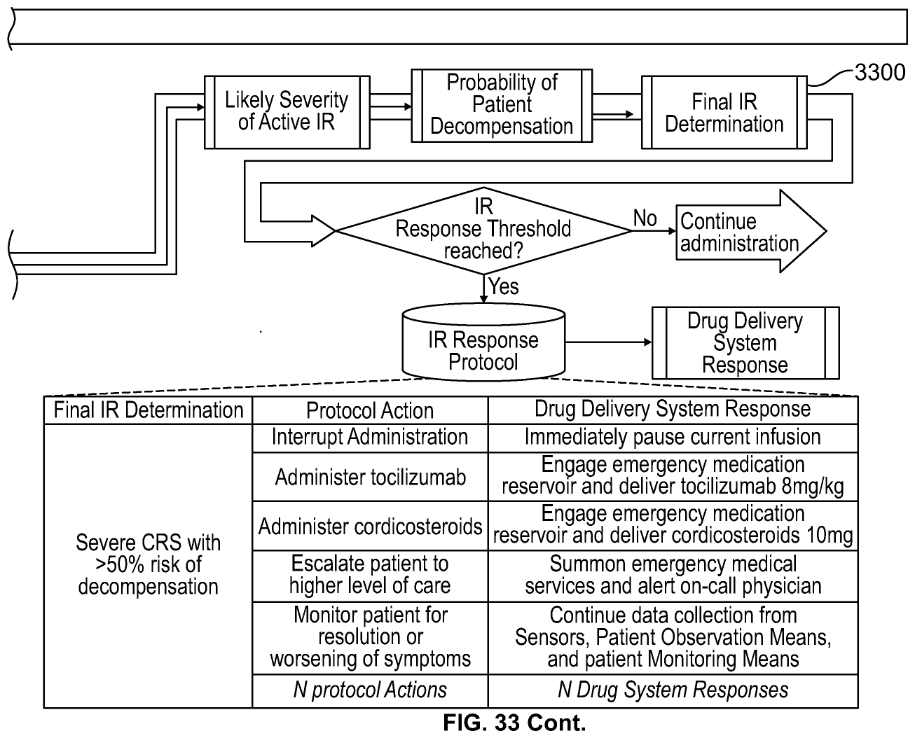

The system depicted in FIG. 31 can be used in the following manner, which is schematically illustrated FIGS. 32 and 33. Predicting the overall infusion reaction (IR) risk, both incidence and severity, prior to drug administration is preferably performed using the IR prediction algorithm(s) contained and executed within the controller in the first subcomponent depicted in FIG. 31. Execution of the algorithm involves inputs include patient anatomic and physiologic data, patient signs, symptoms, and self-report, extracted drug-, disease-, and patient-specific data, and drug delivery device data, collected and communicated by the sensors, external data Sources, and/or the drug delivery device, all in communication and processed by the controller. The IR prediction algorithm outputs can include an IR incidence risk and IR severity risk result.

A next step could include determining the most likely IR subtype, again prior to the actual medication administration. This determination could use an IR typing algorithm(s) that processes inputs that include patient anatomic and physiologic data, patient signs, symptoms, and self-report, extracted drug-, disease-, and patient-specific data, and drug delivery device data, collected and communicated by the sensors, external data sources, and/or the drug delivery device, all in communication with and again processed by the controller. The IR typing algorithm outputs include IR subtype probabilities and subsequently a determination of the most likely subtype 3200 (see FIGS. 32).

As illustrated in FIG. 33, a next step could include detecting an occurring IR during or after administration using an IR detection algorithm(s) that is resident and executed in the controller. Inputs used by this algorithm include patient anatomic and physiologic data, patient signs, symptoms, and self-report, extracted drug-, disease-, and patient-specific data, and drug delivery device data, collected and communicated by the sensors, external data sources, and/or the drug delivery device, all in communication with and again processed by the controller. This IR detection algorithm will generate outputs that include probability of active IR by subtype, likely severity of active IR, and probability of patient decompensation due to occurring IR, and a final IR determination 3300.

A final step in the method could include initiating the optimal IR response during or after administration using IR response protocols. Protocol inputs include outputs from the prediction, typing, and detection algorithms mentioned above, and IR response thresholds, which may be preset for a specific drug, patient population, individual patient, or scenario, and protocol actions, which may be preset for specific drug, patient population, individual patient, or scenario. protocol outputs can include corresponding drug delivery system responses according to inputs, which may include closed loop drug administration, continued or extended monitoring, or notification of third parties (e.g., HCPs).

In addition to above-described methods for predicting, typing, detecting, and responding to IRs for individual patient scenarios, the present disclosed system has other applications. For example, in an alternative embodiment, data collected and used by the drug delivery system and external data sources can be selectively aggregated to identify the “IR fingerprint” (i.e., specific characteristics associated with incidence and severity of IR) for different groups. Data can be aggregated according to a particular drug (e.g., rituximab), dosing strategy (e.g., fixed-dose vs. variable-dose), route of administration (e.g., intravenous vs. subcutaneous), class (bispecific T-cell engager), or target/mechanism of action (e.g., CD20). Similarly, data can be aggregated for patients with particular disease states (e.g., diffuse large B cell lymphoma, or more broadly, lymphoma), characteristics (e.g., clinical stage, high tumor burden, experienced with multiple lines of therapy), demographics (e.g., female patients, patients >65 years old), or any combination thereof. Data can be also aggregated longitudinally to identify the IR fingerprint for an individual patient or even more granular, an individual patient treated with a specific drug. An additional benefit is that any of these models used to identify IR fingerprints will improve over time as more test data is aggregated.

Once an IR fingerprint is identified, response protocols can be adjusted accordingly to create tailored protocol actions and drug delivery system responses for given scenarios prospectively. This can be used in clinical practice once IR-inducing medications are already marketed but can also be used during clinical development to determine the optimal Response Protocols prior to drug approval, including whether certain precautions are required (e.g., pre- or post-medications to reduce the risk of IR), which site of care (i.e., home vs. clinic) is safest/most appropriate, and what level of supervision (i.e., presence vs. absence of direct HCP observation) will be necessary. Even in the absence of closed-loop device functionality, this application could help healthcare facilities establish more precise IR protocols for specific drugs and patient populations. IR fingerprinting also offers value in development to establish better correlations between molecule characteristics (e.g., certain structures, moieties, residues, side chains, or manufacturing techniques) and propensity to cause IRs, potentially guiding drug developers away from problematic molecules a prior. Finally, IR fingerprinting can be applied during pre-clinical development to determine a drug's safety to administer in human studies, as absence of precise pre-clinical signals has resulted in unexpected and tragic IR consequences for patients in early clinical trials.

Yet another alternative application of the above-described system enables standardized collection of IR events and associated data, both in clinical practice and clinical development. Currently, IR diagnosis relies on subjective patient assessment and clinical judgement, which makes precise evaluation of IR prevalence, severity, and differentiation extremely difficult. Using data-driven algorithms to predict, type, and detect IRs would minimize the subjectivity and variability inherent to the current process and allow IRs to be studied (in clinical development) and characterized (in clinical practice) in a precise and standardized fashion. The same is true for standardizing IR response, which is also currently highly variable in practice.

A further application of the current disclosure enables IR response thresholds to be established based on probability (e.g., sensitivity and specificity, number needed to treat vs. harm) or be pre-defined at the discretion of a third party (e.g., a healthcare provider or pharmaceutical manufacturer). For example, it could be the case that the benefit of treating very frequent but mild IRs with closed-loop emergency medication outweighs the risk of harm from a purely probabilistic standpoint. However, that IR response threshold may not be deemed appropriate from a patient preference (e.g., unnecessary medication administration), HCP practicality (requirement to intervene), or cost (e.g., “wasting” medication) standpoint. Instead, it may be preferable to allow certain stakeholders to predefine IR response thresholds that take these factors into consideration. In this way, stakeholders can have the flexibility to determine what is an acceptable risk-benefit ratio in different scenarios in a similar manner to a failure mode and effects analysis.

### Infusion Reaction Response

Contingent administration of emergency medications is particularly provided by the present disclosure, allowing safe administration of medications with propensity to cause side effects or reactions.

FIG. 2E illustrates an exemplary drug delivery apparatus or system configured to contingently administer certain emergency medicines to counteract symptoms and/or to treat systemic infusion reaction caused by administration of one or more therapeutic medications also contained within the system.

In a first alternative embodiment, the drug delivery system is configured to administer one or more emergency medications using the same tubing set lumen used to administer one or more therapeutic medications. A drug delivery apparatus or system 285 is provided with a reservoir 286 for a therapeutic medication 287, a reservoir 288 containing an emergency medication 289, and fluidic connections 290, 291 between the reservoirs and a fluid pump 292, and a single lumen tubing set 293 fluidically connected between the fluid pump 292 and patient interface 295. Medication 287 is administered to the patient. In accordance with the disclosure herein, in the case of a suspected or actual infusion reaction, the emergency medication 221 is administered to the patient 294 through the patient interface 295.

In a second alternative embodiment, the drug delivery system is configured to administer one or more emergency medications in a pre-emptive manner using an alternative lumen than that used to administer one or more therapeutic medications. A drug delivery apparatus or system 285 is provided with a reservoir 286 for a therapeutic medication 287, a reservoir 288 containing an emergency medication 289, and fluidic connections 290, 291 between the reservoirs and a fluid pump 292, and a double lumen tubing set 293′ fluidically connected between the fluid pump 292 and patient interface 295. Medication 287 is administered to the patient using a first medication lumen 297 within the double lumen tubing 293′. In accordance with the disclosure herein, in the case of a suspected or actual infusion reaction, flow of the therapeutic medication 287 is halted within the first medication lumen 297 and the emergency medication 221 is administered through a second medication lumen 298 within double lumen tubing 293′ and into the patient 294 through the patient interface 295.

In some embodiments, the emergency medication is administered in response to a suspected systemic infusion reaction triggered by administration of one or more therapeutic medications. In some embodiments, the emergency medication is administered in response to a patient experiencing an adverse event. In some embodiments, the emergency medication is a reversal agent for one or more therapeutic medications.

In some embodiments, the emergency medication is epinephrine. In some embodiments, the emergency medication is naloxone. In some embodiments, the emergency medication is a corticosteroid. In some embodiments, the emergency medication includes one or more medications selected from the group of hydrocortisone, dexamethasone, or methylprednisolone. In some embodiments, the delivery apparatus or system is configured to reconstitute a lyophilized emergency medication prior to administration. In situations where time may be of the essence, the delivery apparatus or system may be configured to reconstitute a lyophilized emergency medication in an anticipatory fashion, such as when a potential infusion reaction is first detected by a sensor, but before administration has been ordered by a healthcare provider.

In some embodiments, the drug delivery apparatus or system is configured to administer an emergency medication automatically based on predetermined physiologic or clinical criteria. In some embodiments, the drug delivery apparatus or system is configured to administer an emergency medication based on instructions from a remote healthcare provider. In some embodiments, the drug delivery apparatus or system is configured to administer an emergency medication based on instructions from a user proximal to the apparatus or system.

### Clinical Trial Configuration

One primary benefit of embodiments of the drug delivery apparatus or systems disclosed herein is to allow commercial presentations of an approved medication to use the same delivery apparatus or system used in earlier clinical studies, without the need to design, validate, or test a second apparatus or system for commercial presentation. The present disclosure increases flexibility to accommodate a wide variety of pharmacokinetic profiles, even if the behaviors are not known in advance.

Pharmacokinetic (PK) profiles as used herein is a term of convenience, but components of PK profiles are well-understood by those skilled in the art and may include, by way of example but not limitation, bioavailability, Tₘₐₓ, Cₘₐₓ, Area Under Curve (AUC), C_{trough}, absorption rate constant, elimination rate constant, half-life, volume of distribution, clearance, and/or steady state concentrations. As used herein, Cₘₐₓ and C_{trough} are the maximum and minimum concentrations a drug reaches in the systemic circulation after administration of a given dose, respectively. Tₘₐₓ is the time required to reach Cₘₐₓ after administration of a given dose.

FIG. 3A depicts a schematic of the preclinical and clinical development processes for dose determination of a typical parenteral drug with the present disclosure incorporated. In this process, the appropriate tubing set or sets 322 to employ in Phase 1 trials is determined in parallel with and influenced by formulation development 320 and pharmacokinetic modeling 321. Notably, the appropriate tubing set or sets, which govern flow desired rate in the clinical study 324, are decoupled from the dose range, which may be varied independently.

Phase 1 clinical trials are then conducted to establish dose ranges in a manner familiar to those skilled in the art. Tubing set(s) 325 as determined in 322 are supplied to the clinical trial site and are used to conduct the initial Phase 1 trial 324 according to desired clinical trial conditions, including the hypothesized dose ranges 323. Analysis of Phase 1 trial 326 data leads to dosing regimen refinement 327 used to design follow-on clinical trials.

If regimen refinement yields only a single dosing regimen 328, a single appropriate tubing set 330 will be designed for use in Phase 2 studies 330, corresponding to the desired clinical trial condition 339 from pharmacokinetic data and dose evaluation 326. If regimen refinement yields multiple possible dosing regimens 332, one alternative embodiment of the current disclosure provides for an appropriate kit of one or more tubing sets 333 to be designed for use in Phase 2 studies 334, wherein the kit components each correspond to one or more clinical trial conditions 336, 337, or 338.

Once the desired efficacy signal 340 is achieved with one or more dosing regimens, the appropriate tubing set or sets are determined 341 for the Phase 3 clinical trial, and then used in the Phase 3 trial 342 based on prior clinical trial results and corresponding to the desired clinical trial condition. Finally, upon regulatory approval, the appropriate tubing set or sets are selected for the commercial product 344 based on pivotal clinical trial results and the desired commercial presentation.

In an embodiment, during one or more clinical trials, staff select one or more tubing sets from a subset in Phase 1 324 and Phase 2 330 and 334 studies, then select a smaller subset of tubing sets for Phase 3 342 studies. In some embodiments, a smaller subset of tubing sets than those used for Phase 3 342 studies are provided to patients in a commercial presentation of the approved medication. In some embodiments, the same tubing sets used for Phase 3 342 studies are provided in a commercial presentation of the approved medication.

One advantageous aspect of the present disclosure is flexibility to accommodate use in both clinical trials and commercially marketed medications. Special considerations apply to drug delivery apparatus or systems used in clinical trials. Clinical trial data should be accurate, traceable, and reproducible; thus, data integrity is a cornerstone of successful clinical research and is an ethical and regulatory requirement designed to allow confident decision-making regarding approval of medicines.

Clinical trials take place in many different settings, depending on the clinical study phase, specific medication, and patient population. For instance, referring again to FIG. 3A, many Phase 1 studies 304 and Phase 2 studies 309 and 310 are completed at clinical trial sites or in clinic. Phase 3 studies 313 may be completed at clinical trial sites, in clinic, or in the home setting. For Phase 3 studies 313 completed at home, alternative embodiments of the present disclosure are especially advantageous when configured to improve clinical trial data integrity through the incorporation of sensors, controllers, and permanent data storage meeting GCP or other regulatory requirements in a variety of configurations.

Referring to FIG. 8, in an alternative embodiment, the drug delivery system 775 includes one or more sensors 782 coupled to a controller 779 to measure patient 783 vital signs at one or more stages before, during, and after administration of one or more therapeutic medications studied within a clinical trial 776. As medication administration progresses, data from the physiologic sensors 783 are recorded into permanent data storage 785 for later retrieval and analysis 787 by a clinical trial team 784. This provides later analysis of data by the clinical trial team 784 to identify any potential propensity for infusion reactions or other adverse physiologic effect as a result of the medication studied within the clinical trial 776.

In an alternative embodiment, the drug delivery system 775 includes one or more sensors 782 to measure the status of medication administration at one or more stages before, during, and after administration of one or more therapeutic medications studied within a clinical trial 776. As medication administration progresses, sensor 382 data are communicated 781 to a controller 779 and transferred 786 to permanent data storage 785 for later retrieval and analysis 787 by a clinical trial team 784. This provides for later analysis of data by the clinical trial team 784 and verification that each patient received a full medication dose as expected. In an alternative embodiment, the sensors may also be provided on one or more medication reservoirs 776′ containing an investigational therapeutic medication 776.

In an alternative embodiment, the drug delivery system 775 includes one or more sensors 782 to monitor the patient interface throughout administration of one or more investigational therapeutic medications studied within a clinical trial 776. The sensor 782 data are communicated 781 to a controller 779 and transferred 786 to permanent data storage 785 for later retrieval and analysis 787 by a clinical trial team 784. This provides for later analysis of data by the clinical trial team 784 and verification that the medication was, in fact, administered directly to the patient as intended. In some embodiments, the sensor 782 comprises a skin sensor. In some embodiments, the sensor 782 comprises a flow sensor.

In an alternative embodiment, the drug delivery system 775 includes a controller and algorithm 779 to monitor the state of drug delivery system 775 throughout administration of one or more investigational therapeutic medications studied within a clinical trial 776, further communicating 781 any such detected failures to permanent data storage 785 for later retrieval and analysis 787 by a clinical trial team 784. This provides for later analysis of data by the clinical trial team 784 and verification that the drug delivery system 775 operated as intended during administration of an investigational therapeutic medication 776.

### Electronic Health Record Integration

Clinical trials occur in highly controlled settings to minimize confounding variability that could affect data integrity and mask positive or negative pharmaceutical efficacy. Once an investigational therapeutic medication is approved, administration may take place at home, in clinic, or both. In day-to-day patient care, treatment of diseases may be complex, necessitating the coordination of multiple medications, lab tests, and physical visits with a healthcare provider. Health-related information is often stored in an electronic health record (EHR), wherein patient information is centrally stored and accessible to authorized users, such as the patient's doctors, nurses, and pharmacists. By including EHR integration as described herein, the present disclosure provides continuity of care between the clinic and home, which is crucial when medications are given in both settings, as is true in the case of a medication regimen, such as for oncology.

EHRs may also contain orders, which are instructions to care for, diagnose, and treat each patient. Referring to FIG. 10, in one or more embodiments, the drug delivery system 1020 is provided with a controller 1026 that communicates with components of the apparatus via either a wired or wireless connection. In one or more embodiments, the controller according to one or more embodiments comprises a processor 1023, a memory coupled to the processor 1024, input/output devices 1025 coupled to the processor 1023, an EHR interface 1021 coupled to the processor, and support circuits to provide communication between the different components of the system, namely the components of the system described herein. In one or more embodiments, processes to operate the system are stored in the memory 1024 as a software routine that, when executed by the processor, causes the system to perform methods described in the present disclosure. In one or more embodiments, the processes to operate the system are performed in hardware. In one or more embodiments, the software routine to operate the system may also be stored and/or executed by a second processor that is remotely located from the hardware being controlled by the processor.

In some embodiments, the EHR interface 1021 is implemented with a Wi-Fi, wireless local area network (WLAN), Bluetooth, near field communication (NFC), cellular, or internet protocol (IP) connection. In some embodiments, redundant input/output interfaces are provided if one communication interface fails. In some embodiments, the EHR interface 1021 features end to end encryption. In some embodiments, the EHR interface 1021 interface is implemented with an application programming interface (API).

The drug delivery apparatus or system 1020 interfaces with an EHR system 1000 via EHR interface 1021 and is thereby associated with one or more specific medication orders 1001 related to a therapeutic medication 1027. In some embodiments, the association includes corresponding order parameters 1007 and administration time 1008 for a therapeutic medication 1002. In some embodiments, the drug delivery apparatus or system is associated with one or more specific medication orders 1001 contained within EHR system 1000 via EHR interface 1021 and corresponding order parameters contained within the EHR system, wherein the order parameters include an identifying number 1005, prescriber 1009, medication name 1002, and administration parameters 1007 and time 1008. In some embodiments, the drug delivery apparatus or system 1020 is associated with one or more specific medication orders 1030 (shown in FIG. 10B) contained in one or more order sets 1030 contained within the EHR system 1000 via EHR interface 1021.

Order sets may also be provided in EHR systems, comprising aggregation of multiple orders related to a single condition, process, or clinical situation, such as administration of one or more therapies to treat a disease. In some embodiments, the drug delivery system interfaces with an EHR system 1000 via EHR interface 1021 and is thereby associated with one or more specific medication orders 1001 contained in one or more order sets 1030 within an EHR system 1000, wherein the order sets contain administration instructions for one or more therapeutic medications 1032, medications given prior to 1031 and after 1033 one or more therapeutic medications 1032, and/or standing orders related to emergency medication administration 1033. In some embodiments, the drug delivery apparatus or system 1020 is associated with one or more specific medication orders 1001 contained in one or more order sets 1030 within an EHR system 1000, wherein the order sets contain physiologic monitoring instructions 1037 for a given patient.

Prior to administration, orders and order sets are also used in clinical practice to dispense medications to specific patients, and to verify that the proper medicines are dispensed to each patient. In some embodiments, referring to FIG. 10C, the drug delivery apparatus or system 1020 is associated with one or more specific medication orders 1001 within an EHR system 1000, and the drug delivery apparatus or system 1020 contains means by which the contents of reservoir 1027′ holding the therapeutic medication 1027 may be verified at 1011 against the order 1001 by a healthcare provider 1010 prior to dispensing to the patient.

In some embodiments, the drug delivery apparatus or system 1020 is associated with one or more specific medication orders 1001 contained in one or more order sets 1030 within an EHR system 1000, wherein the medication orders are referenced on the drug delivery apparatus or system using a barcode or data matrix 1022 that can be scanned by equipment connected to the EHR system 1000.

In some embodiments, the order set 1030 comprises one or more instructions for administration of one or more therapeutic medications 1032, administration of one or more pre-medications 1031 or post-medications 1032 related, administration of one or more emergency medications 1033, required laboratory values or patient monitoring 1034, or other instructions to nursing 1035, 1036, 1037.

In certain cases, administration of a medication may be subject to specific laboratory values being within specified ranges set forth in one or more medication orders 1001 or order sets 1030. Review of laboratory values may be performed manually by a healthcare provider, or through automated decision support within the EHR system. In some embodiments, the drug delivery apparatus or system 1020 is associated with one or more specific medication orders 1001 contained in one or more order sets 1030 within an EHR system 1000, wherein the order sets permit administration of one or more therapeutic medication(s) 1030 pending review of one or more diagnostic or laboratory criteria 1035 contained elsewhere in the EHR system 1000, wherein the review is completed by a healthcare provider. In some embodiments, the drug delivery apparatus or system 1020 is associated with one or more specific medication orders 1001 contained in one or more order sets 1030 within an EHR system 1000, wherein the order sets permit administration of one or more therapeutic medication(s) 1030 pending review of one or more diagnostic or laboratory criteria 1035 contained elsewhere in the EHR system 1000, wherein the review is completed automatically by a decision support tool also contained within the EHR system 1000.

Medication orders and order sets provide administration instructions, including administration rates. So-called “hard” limits cannot be overridden by a healthcare provider, while so-called “soft” limits may be overridden by a healthcare provider based on professional judgment. Embodiments of the present disclosure allows both types of limits to be implemented. In some embodiments, the drug delivery apparatus or system 1020 is provided with an EHR interface 1021 and is associated with one or more specific medication orders 1001 within an EHR system 1000, wherein the medication orders and EHR interface prohibit administration of one or more therapeutic medication(s) at parameters that are unsafe or clinically inappropriate, and wherein the prohibition may not be overridden by one or more healthcare providers 1010 in the interest of patient safety. In some embodiments, the drug delivery apparatus or system 1020 is provided with an EHR interface 1021 and is associated with one or more specific medication orders 1001 within an EHR system 1000, wherein the medication orders and EHR interface prohibit administration of one or more therapeutic medication(s) 1027 at parameters 1007 that are unsafe or clinically inappropriate, and a means for one or more healthcare providers 1010 to override such prohibition based on clinical judgment.

In some embodiments, the drug delivery apparatus or system 1020 is provided with an EHR interface 1021 and is associated with one or more specific medication orders 1001 within an EHR system 1000, and wherein communication between the EHR interface 1021 and drug delivery apparatus or system 1020 is bi-directional, allowing clinician review 1038 of the order 1001's corresponding parameters and administration progress thereto within the health record system.

In another aspect, the drug delivery system controller herein is provided with an input/output interface to allow communications between the administration location and a remote monitoring service. In some embodiments, all sensor data collected by the drug delivery apparatus or system is communicated to the remote monitoring service by the controller. In some embodiments, a subset of sensor data collected by the drug delivery apparatus or system is communicated to the remote monitoring service by the controller. In some embodiments, the remote monitoring service is manned by a healthcare provider. In some embodiments, the remote monitoring service is a computing apparatus or system. In some embodiments, the remote monitoring service is a healthcare provider aided by a decision support tool implemented in software. In some embodiments, the decision support tool employs a predictive or machine learning algorithm. In some embodiments, the decision support tool is an electronic health record (EHR) system.

In some embodiments, the drug delivery system is programmed based on an order set to monitor specific vital signs contained in one or more orders contained in an order set. In some embodiments, the drug delivery system is programmed to deliver specific therapeutic medications based on an individual medication order or orders contained within an order set. In some embodiments, the drug delivery system is programmed to allow delivery pending availability of certain laboratory test results contained within the EHR. In some embodiments, the drug delivery system is programmed to allow delivery only upon confirmation from the EHR that certain laboratory values are within predefined ranges. In some embodiments, the drug delivery system is programmed to prohibit delivery if certain laboratory values contained within an EHR are unavailable or outside predefined ranges. In some embodiments, the drug delivery system is programmed to prohibit delivery if certain laboratory values contained within an EHR are unavailable or outside predefined ranges unless the prohibition is overridden by a healthcare provider. In some embodiments, the drug delivery system is programmed to prohibit delivery if certain laboratory values contained within an EHR are unavailable or outside predefined ranges unless the prohibition is removed automatically by a decision support tool contained within the EHR.

One or more embodiments of the disclosure utilize at least one controller which can be coupled to various components of the apparatus and systems as described herein. In some embodiments, there are more than one controller connected to the individual components a primary control processor is coupled to each of the separate processors to control the system or apparatus described herein. The controllers may be one of any form of general-purpose computer processor, microcontroller, microprocessor, etc., that can be used in an industrial setting for controlling various delivery and/or treatment regimens.

A controller can have a processor, a memory coupled to the processor, input/output devices coupled to the processor, and support circuits to provide communication between the different electronic components. The memory can include one or more of transitory memory (e.g., random access memory) and non-transitory memory (e.g., storage). The memory, or computer-readable medium, of the processor may be one or more of readily available memory such as random-access memory (RAM), read-only memory (ROM), floppy disk, hard disk, or any other form of digital storage, local or remote. The memory can retain an instruction set that is operable by the processor or controller to control parameters and components of the apparatus and methods described herein. The support circuits are coupled to the processor for supporting the processor in a conventional manner. Circuits may include, for example, cache, power supplies, clock circuits, input/output circuitry, subsystems, and the like.

Processes and methods such as treatment regimens may generally be stored in the memory as a software routine that, when executed by the processor, causes the apparatus and systems described herein to perform methods described in the present disclosure. The software routine may also be stored and/or executed by a second processor (not shown) that is remotely located from the hardware being controlled by the processor. Some or all of the method of the present disclosure may also be performed in hardware. As such, the process may be implemented in software and executed using a computer system, in hardware as, e.g., an application specific integrated circuit or other type of hardware implementation, or as a combination of software and hardware. The software routine, when executed by the processor, transforms the general-purpose computer into a specific purpose computer (controller) that controls the chamber operation such that the processes are performed.

In some embodiments, the controller has one or more configurations to execute individual processes or sub-processes to perform the methods described herein.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, those skilled in the art will understand that the embodiments described are merely illustrative of the principles and applications of the present disclosure. It will be apparent to those skilled in the art that various modifications and variations can be made to the method and apparatus of the present disclosure without departing from the spirit and scope of the disclosure. Thus, the present disclosure can include modifications and variations that are within the scope of the appended embodiments and their equivalents.

In another aspect, embodiments of the disclosure generally relate to apparatus, systems and methods for administration of therapeutic medicines. More particularly, embodiments of the disclosure relate to apparatus, systems and methods configured to control fluid flow within a tubing set within a drug delivery system, especially for biologic medicines.

Liquid medication formulations are commonly administered through tubing sets, and the ability to control flow rates through a tubing set is highly desirable for medication delivery. Many configurations of tubing sets and flow controls are available on the market.

Certain existing devices control flow rate with a pre-defined inner diameter, each corresponding to a flow rate for a given medication. For instance, two tubing sets, each with different internal diameters, may be labeled for 150 mL/h or 300 mL/h when using a specific fluid, such as saline. One disadvantage of this approach is that if an intermediate flow rate is desired (e.g., 200 mL/h), another dedicated tubing set must be manufactured, adding cost and inefficiency.

A kit of components with multiple tubing sets may also be fluidically combined in series or parallel, allowing “mix and match” construction of a set providing a desired flow rate. For example, connection of two 100 mL/h tubing sets in parallel allows construction by a user of a 200 mL/h set, allowing efficient production of a single 100 mL/h set at scale. While efficient from a manufacturing perspective, significant burden is placed onto the user, who must understand the concepts of fluid mechanics sufficiently to select and assemble the correct components in the right combination. This may prove challenging or inconvenient for users without clinical training, as would be present in a home setting. Moreover, it may present medication safety risks that are not apparent to an untrained user. Incorrectly assembled or missing components in such a system will provide flow rates substantially different than intended, leading to over- or under-delivery of medication and potential adverse events. The same risk may also be present if one or more components of the kit is unavailable due to a backorder or product recall. The Institute for Safe Medication Practices error reporting program has documented cases of patient hospitalization and death due to incorrect selection of rate control tubing and corresponding overdose in the home setting.

Another alternative is to provide a variable rate controller on a tubing set, allowing a user to increase or decrease the flow rate during administration, for instance, with an adjustment knob. If a medication is provided with a prescribed administration rate of 50 mL/h, such a device would be provided in the “off” (0 mL/h) state at dispensing, and the user would be required to set the flow rate to 50 mL/h on the rate controller accurately during administration. Healthcare providers in clinical settings are familiar with such devices and can use them safely. In the home setting, this presents potential safety risks to users, who often lack clinical training. They may increase the flow rate beyond that prescribed to complete administration sooner without understanding the pharmacokinetic effects of such an increase. They may misunderstand the instructions on a prescribed medication or for use of the rate control and set the rate incorrectly. In some instances, rate control devices may be provided to simply stop or start flow, even when specific flow rate control is not needed; untrained users may not understand and inadvertently make unintended flowrate adjustments.

Notably, both of these existing tubing set assemblies designed for constant preset flow and a rate controller both provide flow rates that are calibrated on a specific fluid. Most commonly, this fluid is a Newtonian fluid, such as water or saline, in which the viscosity remains constant with shear strain, defined as a proportional constant (coefficient of viscosity).

When used with non-Newtonian medications, different tubing sets and rate controllers may deliver drastically different flow rates. Certain medicines, such as biologics or long-acting injectable (LAI) formulations may display non-Newtonian behaviors, wherein the relationship between viscosity and shear strain may not be defined by a single constant. Shear thickening fluids exhibit increasing viscosity with increasing shear rate, while shear thinning fluids exhibit decreasing viscosity with increasing shear rate.

Biologics may also exhibit strong temperature-viscosity-concentration relationships which are a significant design consideration for drug delivery devices, and fluidic components such as tubing sets specifically. To reduce dosing frequency for patients, pharmaceutical companies desire higher biologic concentrations within a formulation, leading to generally higher viscosity that becomes exponentially more viscous with decreasing temperature. Thus, medications may flow differently in tubing sets with different inner diameters (and thus different shear rates) or fluid restrictors, and warmer or colder biologic medicines may exacerbate these behaviors in a way that is not obvious to an untrained user. In addition, a tubing set labeled for a specific flow rate (e.g., 900 mL/h) with saline, a Newtonian fluid, may deliver a much lower flowrate (e.g., 60 mL/h) with a more viscous non-Newtonian fluid, requiring complex conversion tables that patients and dispensing pharmacists must interpret (and may interpret incorrectly). Moreover, these nuances may not be apparent to the patient or dispensing pharmacist, resulting in the incorrect tubing set being dispensed to a patient. These shortcomings may result in dangerous under- or over-doses, with particularly deleterious safety and efficacy implications for administration of potent biologic medicines. These limitations with existing devices result in patients using a product that delivers medication differently than labeled. There is a need to avoid this issue.

Moreover, fine-grained control over flowrate provided by existing devices is often unneeded. Many medications have fixed dose regimens, where all patients receive a specific dose at a specific flow rate. Other medications commonly have “dose bands,” where patients receive one of several discrete fixed doses, often based on body weight. Both scenarios are common with biologic medicines, where doses and rates do not vary from administration to administration. In these administration scenarios, the complexity of prior art systems is not only unneeded but also encourages unwanted or unsafe adjustment, particularly by a user without clinical training or with low health literacy.

Embodiments provide apparatus, systems and methods for administering large volumes of parenteral or enteral medicines at one or more predetermined rates, particularly non-Newtonian medicines exhibiting a concentration-viscosity-temperature relationship, such as a biologic medicine.

Before describing several exemplary embodiments of the disclosure, it is to be understood that the disclosure is not limited to the details of construction or process steps set forth in the following description. The disclosure is capable of other embodiments and of being practiced or being carried out in various ways.

The required dosing and associated flow rate of therapeutic medicines are intended to induce a specific physiologic effect in the patient receiving the medication. However, especially in the case of biologic medicines, the concentration, dose volume, and dosing rate are finalized through human clinical trials. Thus, dosing parameters needed to design delivery components, including a tubing set, are likely not be finalized until late in the drug development process after human clinical trial data becomes available. Given the non-Newtonian behavior of biologic medicines, changes to these parameters may require substantial redesign of fluidic components, including tubing sets. Thus, improved apparatus, systems and methods are needed to provide flexibility in clinical trial design and execution, and to accommodate late-stage formulation or drug concentration changes without the time and cost associated with a full redesign of the administration components. The present disclosure fulfills one or more of these needs.

Existing devices are focused on tubing sets designed to be used with a variety of different medicines and provide solutions that are inefficient and impractical to implement in a tailored fashion for a specific medication. The onus is placed on the user of existing solutions to adapt to the apparatus available. Instead, ready-to-use administration supplies providing accurate, discrete flow rates for medications with fixed-dose or dose-banded administration are provided by embodiments of the present disclosure. At the same time, manufacturing processes according to one or more embodiments disclosed herein allow for efficient high-volume manufacture of tubing sets tailored to a specific medication and one or more administration conditions, without placing undue burden on an untrained user or compromising medication safety as required by prior art solutions.

Finally, in many cases, it is desirable to administer numerous medications, especially as part of a medication regimen. Individual medications are often part of a larger regimen of medicines, with standardized regimens corresponding to a specific disease state, treatment regimen, or medication. A regimen may also include one or more pre-medications, post-medications, or emergency medications. Each of the foregoing may be administered to a patient at a flow rate different than a therapeutic medication, or on a contingent basis, as in the case of an emergency medication administered contingently upon detection of a systemic infusion reaction. Many existing solutions are limited to apparatus or kits of components that adjust flow rate of a single medication, but do not allow independent adjustment of flow rates for multiple medications. Embodiments of the present disclosure meet one or more of these needs.

Embodiments of the disclosure provide apparatus, systems and methods for administering parenteral or enteral medicines to a patient via a tubing set at one or more predetermined rates. The apparatus or system allows for different configurations of tubing sets to effectuate medication administration to a patient. More specifically, one or more embodiments of the present disclosure provides a device and approach to make a single tubing set, capturing the manufacturing efficiencies at scale, and then constrain the tubing set during manufacture to one or more discrete, preset rates that the end user cannot modify. While the tubing set may be used for a variety of medicines, the constraints may be easily and precisely molded for a specific medication and flow rate, and when provided with indicia as disclosed herein, provide a true flow rate for the specific medication, avoiding mix-ups, confusion, and medication errors. Embodiments of the present disclosure is particularly applicable to biologic medicines that exhibit non-Newtonian behaviors, such as biologic medicines, that are administered by untrained users in a home setting to treat chronic diseases.

One or more embodiments of the disclosure is directed to providing apparatus, systems and methods to constrain a tubing set and one or more internal medication lumens situated therein/thereby reducing the flow rate to a specific predetermined rate. One or more constraints as described in embodiments herein are applied to the exterior of a tubing set, reducing the flow rate in one or more lumens of the set, and establishing a maximum flow rate for a lumen so constrained. Geometry of the constraints is matched to a specific therapeutic medication and one or more desired flow rates of the medication. The constraining profile in contact with the tubing set is fashioned with a generally rounded section to provide a gradual reduction of flow from the unconstrained inner diameter to the constrained one, which is particularly advantageous to avoid damage to a therapeutic protein. The length of the constraint may also be varied in conjunction with the constraint profile to enable a gradual reduction in flow. The design of the constraint is such that manufacturing and assembly may be conducted in an automated, efficient manner.

Further embodiments of the disclosure are directed to providing apparatus, systems, and methods to constrain a tubing set and one or more internal medication lumens situated therein, thereby reducing the flow rate to one of several specific predetermined rates during assembly of the constraint apparatus. In one or more embodiments, an adjustable constraint apparatus is provided with one or more discrete adjustment levels corresponding to one or more levels of progressively compressive assembly, successively reducing the flow rate in or more lumens of the set, wherein each distinct compression step corresponds to one flow rate of a specific medication at a specific administration condition, such as room temperature. In one or more embodiments, the constraints are designed to be adjustable in a manufacturing facility by automated equipment while preventing inadvertent or intentional adjustment by a user afterwards.

Additional embodiments of the disclosure are directed to providing apparatus, systems, and methods to accommodate administration of a variety of non-Newtonian biologic medications at a variety of flow rates. In the case of a medication exhibiting a concentration-viscosity-temperature relationship, such as a biologic medication, the flow rates provided by the constraint may optionally be calibrated to provide an intended flow at a specific temperature corresponding to the anticipated medication administration conditions, such as room temperature. In one or more embodiments, the constraint apparatus described herein may be calibrated to one or more desired flow rates during manufacturing for a specific therapeutic medication of interest, such as a biologic medication.

In one or more embodiments, one or more inner medication lumens constrained as described herein inherently limit allowable flowrates through the system at or below the clinically safe or tolerable of one or more medications administered through one or more of the lumens in the tubing set. In some embodiments, the constraint establishes a maximum flow rate for a single internal tubing lumen. In some embodiments, the constraints are placed to establish different maximum flow rates for a two or more internal tubing lumens independently. In one or more embodiments, the constraint apparatus includes an emergency clamp to completely restrict flow or allow flow at the maximum rate provided by the constrained tubing set lumen.

As described herein, the constraints are preferentially injection molded, providing tight dimensional control over the constraint geometry and enabling low-cost manufacturing and efficient assembly. In one or more embodiments, the constraint is designed symmetrically, allowing a single part to be used in pairs during an assembly operation. In one or more embodiments, the constraints are provided with an assembly force intentionally exceeding the physical ability of a human, allowing adjustment by manufacturing and assembly equipment while preventing unintended or inadvertent adjustment by an end user.

To improve intuitiveness for untrained users, especially in the home setting, in one or more embodiments, the constraints may be provided with human readable indicia regarding the drug name, flow rate, and other relevant information to avoid medication errors and improve intuitiveness for untrained users. In one or more embodiments, the constraints may also be provided with machine-readable indicia, such as a data matrix or NFC chip, to enable automated inspection and ensure that a therapeutic medication is provided only with the correct corresponding tubing set once constrained as described herein. In one or more embodiments, the constraint members are provided with contours or indicia to allow inspection by automated equipment to verify proper component selection, assembly, constraint level, or flow rate at a given administration temperature.

As will be appreciated by one skilled in the art, there are numerous ways of carrying out the examples, improvements and arrangements of the apparatus, systems and methods disclosed herein. Although reference will be made to the exemplary embodiments depicted in the drawings and the following descriptions, the embodiments disclosed herein are not meant to be exhaustive of the various alternative designs and embodiments that are encompassed by the present disclosure.

Embodiments of the constraint apparatus systems and methods described herein are for use with a tubing set featuring one or more inner medication lumens for administration of medicines to a patient as part of a drug delivery system. During use of a drug delivery system, a first end of the tubing set constrained as described herein is in fluidic communication with a drug delivery system, and a second end of the tubing in fluidic communication with a patient interface that delivers medications contained within the drug delivery system to the patient. In some embodiments, the patient interface comprises a subcutaneous, intramuscular, or intravenous needle set. In some embodiments, the patient interface comprises a Huber needle for accessing an implanted intravenous port. In some embodiments, the patient interface comprises a luer taper, Luer-Lok® connector, luer-activated valve, luer-activated septum, or other luer-activated access device. In some embodiments, the patient interface comprises a threaded or snap-fit non-luer connector.

Each of the embodiments described with respect to FIGS. 14-17C and FIGS. 28-30B may be combined with the embodiments described in FIGS. 1-1C and the corresponding claims. Thus, the embodiments described with respect to FIGS. 14-17C and FIGS. 28-30B may be incorporated or combined with the apparatus, systems and methods of numbered embodiments below.

FIG. 14A shows an alternative exemplary embodiment of an external constraint apparatus to constrain a tubing set for a drug delivery system to a single flow rate. In one or more embodiments, the fully assembled apparatus comprises an interlocking set comprising a first constraint member 1401 and second constraint member 1402. The first constraint member 1401 contains a first contoured constraint profile 1403 that compressively engages the exterior of a tubing set 1405 when assembled to the second constraint member 1402. Likewise, the second constraint member 1402 contains a second contoured constraint profile 1404 that compressively engages the exterior of a tubing set 1405 when assembled to the first constraint member 1401. In one embodiment, the locking mechanism comprises a set of mating locking fingers 1407, 1410 and apertures 1408, 1409, whereby locking finger 1410 is disposed to align with aperture 1408 and locking finger 1407 is disposed to align with aperture 1409, both during a compressive assembly operation 1406, 1406′ with the tubing set 1405 interposed between the constraint members 1401, 1402. In one or more embodiments, the compressive assembly operation 1406, 1406′ comprises two substantially equal opposing forces.

In one or more embodiments, the first and second constraint members 1401, 1402 may be identical, wherein the two constraint members may be assembled when oriented 180° to each other, improving molding, manufacturing, inspection, part handling, and assembly.

FIGS. 14A & 14B illustrate a constraint for a single lumen; however, this concept may be extended to tubing sets with multiple lumens, as illustrated in FIG. 18. A pair of constraint members may be provided in accordance with the prior description with a plurality of contoured segments, each of the contoured segments corresponding to a desired degree of constraint (or no constraint) for a specific tubing lumen. One or more constraint members may be contoured so the constraint may be properly oriented before engaging the outer surface of the tubing set and may optionally be provided with a keyed feature to prevent misorientation prior to assembly, akin to the cooperating apertures and locking fingers described in FIG. 14A.

In more detail, FIGS. 18A and 18B show an example with variable compression of a multi-lumen tube. A first constraint member 1801 and a second constraint member 1802 are shown relative to a tubing set 1805 in FIG. 18A; the tubing set is unconstrained. In this example, the tubing set 1805 comprises three lumens 1806, 1807, 1808, although two, four or more lumens could alternatively be provided. All three lumens could be medication lumens, though the function of the lumens could also be varied, as discussed elsewhere herein. In FIG. 18B, tubing set 1805 of FIG. 18A is shown compressed by the constraint members 1801, 1802. Due to the internal shape of the constraint members 1801, 1802, the lumen 1806 is uncompressed, the lumen 1807 is compressed, and the lumen 1808 is compressed, with the degree of compression of the lumen 1808 being greater than the degree of compression of the lumen 1807. By varying the internal shape of the constraint members, some or all of the lumens can be compressed, and the lumens can be compressed to different degrees. The shape of the compressed lumens can also be varied by varying the internal shape of the constraint members. An example of an alternative internal shape for the first constraint member 1801 is shown in FIG. 18E; in this example, the internal corners are curved to help minimize or avoid damage to the tubing.

FIG. 18C shows another possible internal shape from the first constraint member 1801. In FIG. 18C, another optional feature of the first constraint member 1801 is shown, namely a protrusion 1810 that is configured to engage a corresponding recess 1811 in the tubing set 1805. The keying structure created by the combination of the protrusion 1810 and the recess 1811 can help ensure that the constraint members 1801, 1802 and the tubing set 1805 are correctly aligned. Only one keying structure is shown, but one or more keying structures could be provided. Various other keying structures could also be used, for example with the protrusion on the tubing set and the recess on a constraint member, or with a snap fit. An alternative keying structure is shown in FIG. 18H, with a non-fitting constraint member 1831 shown on one side and a fitting constraint member 1832 shown on the other side. In the example in FIG. 18H, differing positions and shapes of protrusion and recess are used for the keying structures—differing both the shape and position is optional, but can help ensure that assembly is carried out correctly.

FIG. 18D shows a top view of the constraint members and the tubing set of FIG. 18A, thereby giving an example of what the structure could look like from the outside. In this example, an optional arrow is provided on the outer surface of the first constraint member 1801 to indicate orientation of the constraint members, which can help with assembly. The smaller arrows in the FIG. 18D show the fluid flow direction in the tubing set when in use.

FIGS. 18E and 18F show another example, with a first constraint member 1821, a second constraint member 1822 and a tubing set 1825, which in this case comprises a single lumen. In FIG. 18E, the tubing set is fully open. In FIG. 18F, the tubing set is compressed. The level of compression can be varied by moving the position of the constraint members relative to one another, but hooks 1827 on the first constraint member 1821 and corresponding recesses 1828 on the second constraint member engage so that once the constraint members have been moved towards one another, they cannot be pulled apart again. This can help ensure that a user cannot tamper with a constraint setting applied by a pharmacist, for example. This can be achieved in various ways, such as with a ratchet and/or snap-fit arrangement.

In one or more embodiments, the first and second constraint members 1401, 1402 comprise different geometries that may be oriented and assembled as described herein. In one or more embodiments, the first and second constraint members 1401, 1402 also comprise assembly features to improve orientation and feeding of the device during high-speed assembly, by way of example with a feeding rail or vibratory bowl/rail feeder system. In one or more embodiments, the assembly features comprise a cross sectional design providing a predictable center of gravity to improve feeding and orientation of the constraints during high-speed assembly. In one or more embodiments, the assembly features comprise one or more slots in one or more exterior surfaces of the constraint members. In one or more embodiments, the assembly features comprise one or more protrusions on one or more exterior surfaces of the constraint members.

After compressive assembly 1406, 1406′, and now referring to FIG. 14B, the first constraint member 1401 and second constraint member 1402 creates a constrained inner diameter 1433 within the interposed tubing set 1405, whereas the portion of the tubing set 1405 outside the constraint members 1401, 1402 remains unconstrained 1414. The degree of constraint provided by assembled constraint members 1401 and 1402 to the tubing set 1405 is dependent on one or more of the geometry of the constraint profiles 1403 and 1404, the orientation of the constraint profiles 1403 and 1404 relative to the locking fingers 1407 and 1410 and the apertures 1408 and 1409, the length of the first constraint member 1412, the length of the second constraint member 1413, the outer diameter of the tubing set 1405, the diameter of the medication lumen 1411, the three-dimensional contact area between the constraint profiles 1403 and 1404 and tubing set 1405, and the material of the tubing set 1405.

For certain medicines, it may be desirable to provide a long, smooth transition from unconstrained to constrained inner tubing lumen to avoid protein damage or aggregation due to shearing effects at the medication-tubing lumen interface. Further, the risk of protein damage or aggregation due to shearing effects may vary based on the degree of tubing constraint. In some embodiments, the length of the constraint members 1412, 1413 parallel to the tubing set lumen 1411 axis may be extended to provide a smooth, gradual decrease in flow from the unconstrained portion 1414 to the constrained portion 1433, thus avoiding damage to or aggregation of protein-based or biologic medicines. In some embodiments, the contour of the constraint members 1403, 1404 may vary independently for each flow rate permitted by the constrained section. In some embodiments, the contour of the constraint members 1403, 1404 is engineered to smooth flow reduction while avoiding damage to or shearing of a protein medication flowing through a tubing set lumen 1411.

In an alternative embodiment shown in FIG. 14B, the first contoured constraint profile 1403 and second contoured constraint profile 1404 may be designed to provide a substantially asymmetric cross-sectional constraint when reflected across the long axis of the tubing set 1405 once assembled as described herein. In an alternative embodiment, the first contoured constraint profile 1403 and second contoured constraint profile 1404 may be designed to provide a substantially symmetric cross-sectional constraint when reflected across the long axis of the tubing set 1405 once assembled as described herein. Although FIG. 14B and the accompanying specification shows an asymmetric constraint profile by way of example, it will be clear to those skilled in the art that any arrangement of constraint profiles may be accommodated in the unassembled constraint members and assembled constraint members, and the foregoing example shall not be construed as limiting the apparatus to an asymmetric constraint upon the tubing set once assembled.

Referring to FIGS. 14A and 14B, locking fingers 1407, 1410 may be alternatively secured into the apertures 1408, 1409 in any suitable manner, including press fit, ultrasonic welding, heat staking, adhesive cured by exposure to ultraviolet light after application, or other suitable adhesives. In another embodiment, a UV adhesive is applied to either or both of the apertures 1408, 1409 or locking fingers 1407, 1410 prior to assembly and cured after compressive assembly as described herein to prevent further movement or inadvertent adjustment of the constraint apparatus after assembly.

Although the tubing set 1405 is shown in FIGS. 14A and 14B as having a substantially circular cross section, the apparatus herein may be readily adapted to other tubing designs and configuration of tubing set cross-sections and variations of lumens and conductors situated therein. The present apparatus may, for example, accommodate elliptical designs with cross-sectional designs featuring one or more medication lumens or other non-fluidic conductors disposed therein. Thus, the illustration of tubing set 1405 and accompanying specification is provided by way of illustration and not limitation.

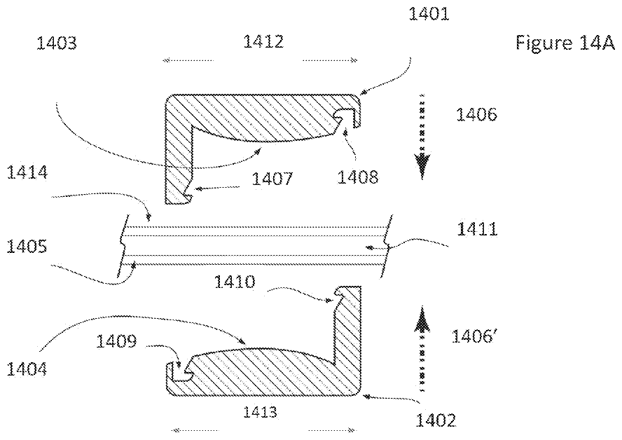

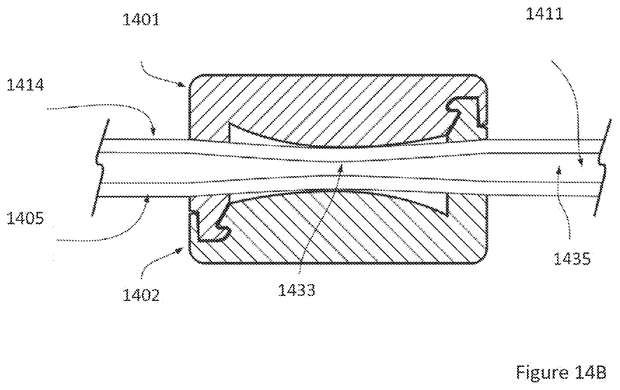

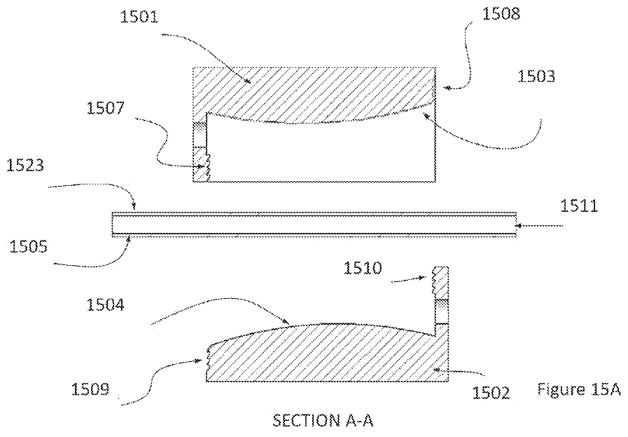

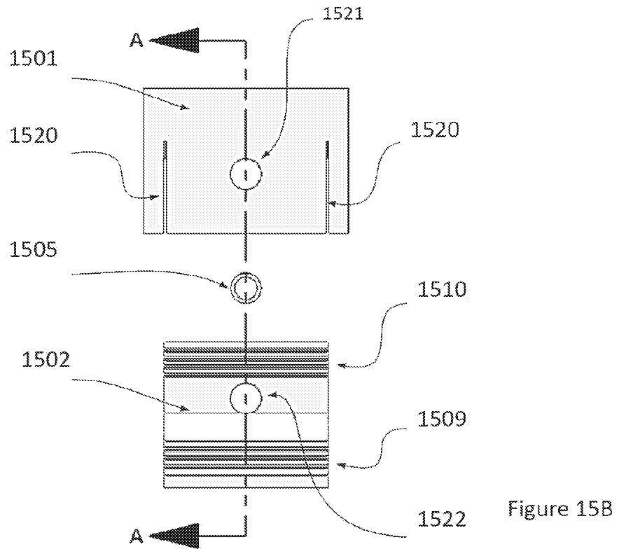

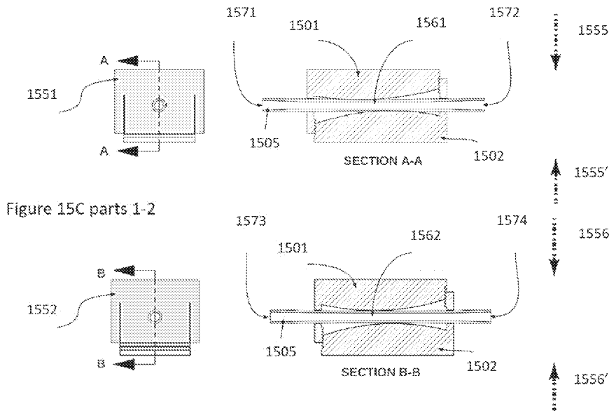

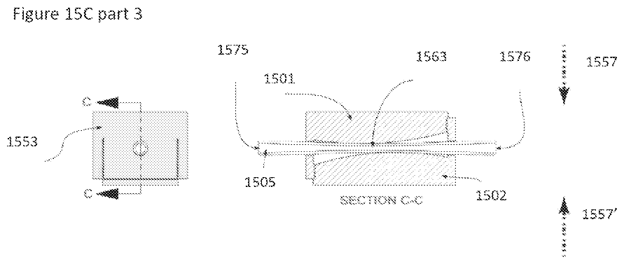

FIGS. 15A and 15B show an alternative exemplary embodiment of an external constraint apparatus to constrain a tubing set for a drug delivery system to one of several discrete degrees of constraint in an adjustable manner during manufacture and assembly while preventing further movement or inadvertent adjustment of the constraint apparatus after assembly.

Referring to FIG. 15A, in one or more embodiments, the fully assembled apparatus comprises an interlocking set comprising a first constraint member 1501 and second constraint member 1502. The first constraint member 1501 contains a first contoured constraint profile 1503 that compressively engages the exterior of a tubing set 1505 with unconstrained diameter 1523 placed through the tubing set entrance 1521 and subsequently assembled to the second constraint member 1502. Likewise, the second constraint member 1502 contains a second contoured constraint profile 1504 that compressively engages the exterior of a tubing set 1505 placed through the tubing set entrance 1522 and subsequently assembled to the first constraint member 1501. In one embodiment, the locking mechanism comprises a set of mating locking fingers 1507, 1510 and apertures 1508, 1509, whereby locking finger 1510 is disposed to align with aperture 1508 and locking finger 1507 is disposed to align with aperture 1509. Once aligned, locking fingers 1507, 1510 are configured to progressively engage with mating apertures 1508, 1509 as the constraints are assembled around an interposed tubing set 1505 using one or more increments of applied compressive forces 1506, 1506′.

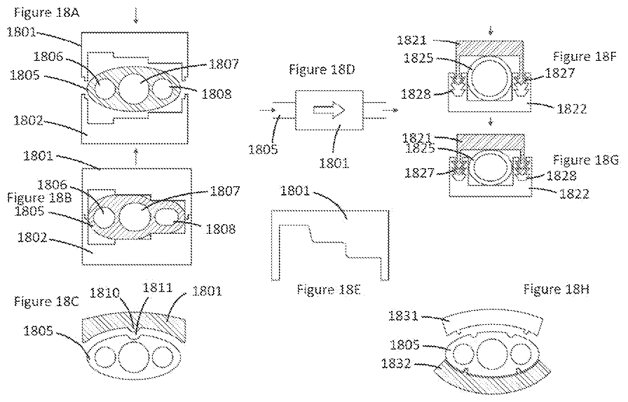

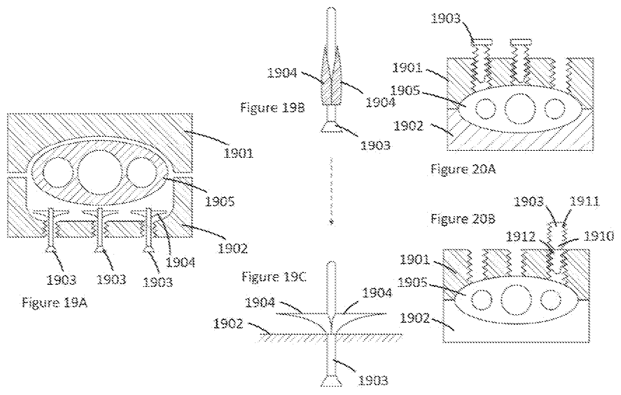

Referring to FIGS. 19 and 20, a ratcheting or threaded plunger may be situated in relationship to a desired tubing lumen to be constrained, wherein each plunger is independently adjustable. The plunger may also have a mechanism to prevent removal of a given constraint once adjusted. As previously mentioned, these examples show a tubing set with three lumens, but other numbers of lumens could also be used.

In more detail, FIG. 19A shows a first constraint member 1901 and a second constraint member 1902 with a tubing set 1905, with the tubing set 1905 optionally having three lumens in this particular example as in the example in FIG. 18A. In this example, three plungers 1903 can be seen; the plungers 1903 extend through the second constraint member 1902. In this way, the different lumens can independently be constrained. Optionally, the level of constraint on one or more of the lumens can be varied dynamically during use. FIG. 19B shows a plunger 1903 with arms 1904 folded away. The arms can then be folded out, as shown in FIG. 19C (and also in FIG. 19A), to prevent removal of the plunger. Optionally, screwing or ratcheting the plunger in to the second constraint member 1902 results in the arms opening.

FIG. 20A shows another option in which the plunger 1903 is simply a screw, again allowing individual manipulation of the compression level of each lumen. FIG. 20B shows another option in which the plunger 1903 has a weak point 1910, so that the outer part 1911 of the plunger breaks off at a set torque, thereby leaving the inner part 1912 of the plunger in a fixed position. This approach can help avoid accidental manipulation of the plunger position after assembly. Breaking apart at a set torque like this can also allow the plunger to finish in a fixed position (at a point where a certain amount of resistance is provided by the squeezed tubing set), thereby potentially simplifying assembly.

Referring to FIG. 15B, the constraint members may optionally be provided with a stress relief 1520 to allow for lower force assembly of the locking fingers and apertures, particularly if a rigid material is selected. The tubing set entrances 1521, 1522 need not fully enclose the tubing set 1505, and one or more entrances 1521, 1522 may be extended centrally towards the tubing set 1505, as in the form of one or more clearance slots, to facilitate more straightforward orientation around the tubing set and subsequent assembly.

The arrangement and spacing of apertures and locking fingers permits constraint of a tubing set to one or more discrete levels using a single set of constraint members, with the constraint level selected during the manufacturing operation. FIG. 15C shows an exemplary illustration of assembly of the apparatus to provide three levels of successively greater constraint upon a tubing set during assembly. Applying a first compressive assembly force 1555, 1555′ to the first and second constraint members 1501, 1502 advances the apertures and locking fingers to a first position 1551, providing a corresponding first degree of tubing constraint 1561 corresponding to a first fixed flow rate. Applying a second compressive assembly force 1556, 1556′ to the first and second constraint members 1501, 1502 advances the apertures and locking fingers to a second position 1552, providing a correspondingly higher second degree of tubing constraint 1562 and correspondingly lower second fixed flow rate compared to the first fixed flow rate at the first degree of constraint 1561. Applying a first compressive assembly force 1557, 1557′ to the first and second constraint members 1501, 1502 advances the apertures and locking fingers to a third (and in this example, final) position 1553, providing a correspondingly still higher third degree of tubing constraint 1563 and correspondingly still lower third fixed flow rate compared to both the first and second fixed flow rates corresponding to the first and second degrees of constraint 1561, 1562. Although FIG. 15C and the accompanying specification shows three positions by way of example, it will be clear to those skilled in the art that any number of positions may be accommodated, and the foregoing example shall not be construed as limiting the apparatus to three degrees of constraint.

In one or more embodiments, the compressive forces 1555 and 1555′, 1556 and 1556′, and 1557 and 1557′ are equal. In one or more embodiments, the compressive forces 1555 and 1555′, 1556 and 1556′, and 1557 and 1557′ are unequal. In one or more embodiments, one or more of the compressive forces 1555 and 1555′, 1556 and 1556′, and 1557 and 1557′ intentionally exceed the compressive force appliable by a human being without the use of mechanical assistance, such as with a tool or fixture. In some embodiments, the compressive force is predetermined, fixed by the design of the constraint members and is configured to prevent modification, tampering, or further adjustment of the compressive force by a user of the apparatus after manufacture and assembly to a tubing set. In one or more embodiments, the third compressive forces 1557 and 1557′ exceed those of the second compressive forces 1556 and 1556′ and the second compressive forces 1556 and 1556′ also exceed those of the first compressive forces 1555 and 1555′.

In one or more embodiments, adjustable constraint members may be constrained adaptively during manufacturing based on the flow characteristics of a medication or medication placebo, which is particularly advantageous when designing fluidic systems for non-Newtonian fluids, such as biologics. Referring to FIG. 15C, a first constraint member 1501 and second constraint member 1502 is applied to a tubing set 1505 through a first compressive assembly step 1555 and 1555′, thus applying a first degree of tubing constraint 1551. During manufacturing, a medication formulation may be passed from the medication inlet 1571 to a medication outlet 1572, measuring the flow rate at both points, and comparing the difference in flow at the outlet 1572 to the desired flow rate. If the desired flow rate at outlet 1572 is within the tolerance of the expected flow rate, adjustment of the constraint members is completed. If the desired flow rate at outlet 1572 is lower than the expected flow rate, a second compressive assembly step 1556 and 1556′ occurs during manufacturing, thus applying a second degree of tubing constraint 1552. A medication formulation is again passed from the medication inlet 1573 to a medication outlet 1574, measuring the flow rate at both points, and comparing the difference in flow at the outlet 1574 to the desired flow rate. If the desired flow rate at outlet 1574 is within the tolerance of the expected flow rate, adjustment of the constraint members is completed. If the desired flow rate at outlet 1574 is lower than the expected flow rate, a third compressive assembly step 1557 and 1557′ occurs during manufacturing, thus applying a third degree of tubing constraint 1553. A medication formulation is again passed from the medication inlet 1575 to a medication outlet 1576, measuring the flow rate at both points, and comparing the difference in flow at the outlet 1576 to the desired flow rate. If the desired flow rate at outlet 1576 is within the tolerance of the expected flow rate, adjustment of the constraint members is completed. If the desired flow rate at outlet 1576 is lower than the expected flow rate, additional compressive assembly and flow testing cycles may be completed as described herein. Although FIG. 15C and the accompanying specification shows adaptative adjustment during manufacturing for three positions by way of example, it will be clear to those skilled in the art that any number of positions may be accommodated, and the foregoing example shall not be construed as limiting the apparatus to three degrees of constraint during the adaptive manufacture described herein. In one or more embodiments, a first constraint member 1501 and second constraint member 1502 are provided with a plurality of apertures and locking fingers, the plurality comprising a number larger than the number of anticipated compressive cycles to achieve a desired flow rate as described herein.

The apparatus described herein provides a consistent flow rate based on one or more degrees of constraint placed upon a tubing set. It may also be advantageous in certain administration settings to provide the constraint with a slidable clamp to completely halt fluid flow, as in the case of an emergency or device malfunction. FIG. 3 shows an alternative embodiment of an external constraint feature that is adjustable to one of several discrete degrees of constraint as described previously, further configured to allow emergency shutoff of a tubing set to prevent any and all flow through the lumens therein.

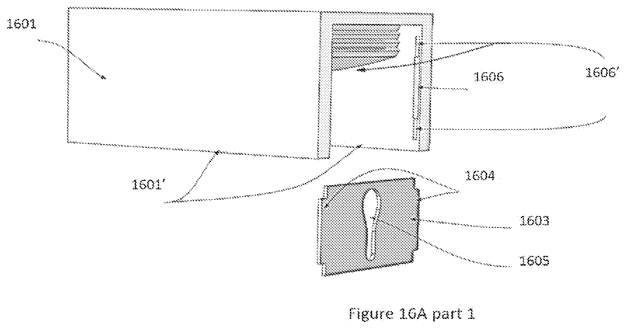

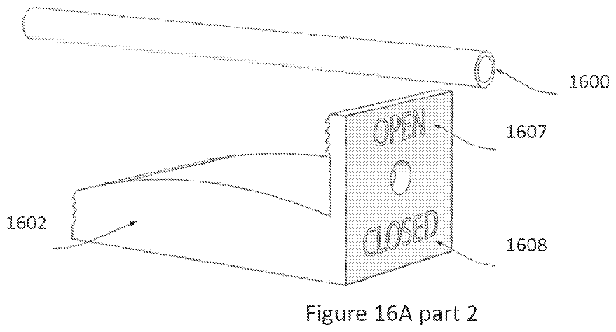

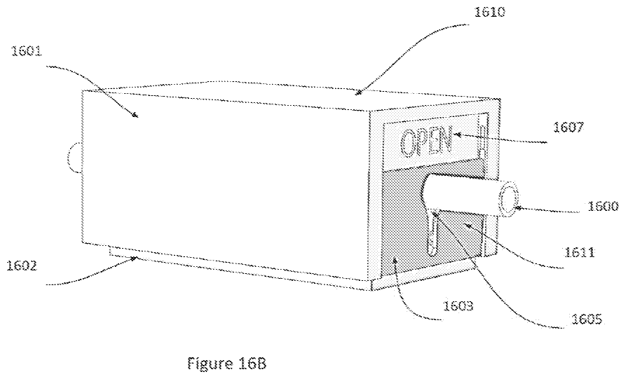

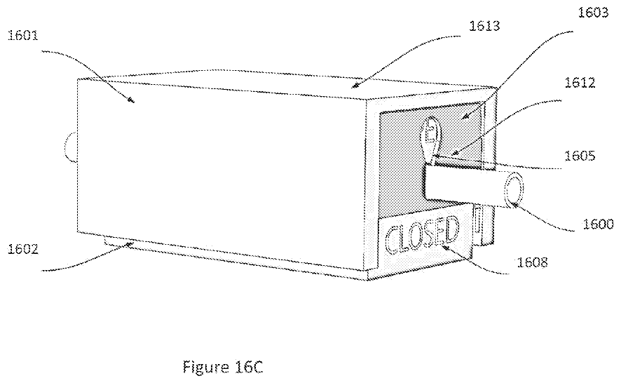

Referring to FIG. 16A, a first constraint member 1601 and second constraint member 1602, each with internal constraint profile, locking fingers, and apertures are provided as described previously herein (refer to FIGS. 15A-15C), and serving to constrain a tubing set 1600 interposed and compressed between the two assembled constraint members 1601 and 1602, also as described previously. Also captured between the first constraint member 1601 and second constraint member 1602 is a clamp plate 1603 comprising one or more travel tabs 1604 and a clamp profile 1605. Clamp travel tabs 1604 are situated to slidably engage with a clamp travel slot 1606 provided in one or more open arms 1601′ of the first constraint member 1601, with the tubing set passing through both constraint members 1601, 1602 and the clamp profile 1605 during assembly. Slidably engaging the clamp plate 1603 within the travel slot 1606 between the clamp travel stops 1606′ allows flow in the tubing to be fully stopped or started depending on the position of the tubing set 1600 within the clamp profile 1605.

FIG. 16B shows a constraint assembly 1610 comprising first constraint member 1601 and 1602 assembled to constrain a tubing set 1600, the constraint members also capturing a slidable clamp plate 1603 with a tubing set 1600 passing through clamp profile 1605. In one or more embodiments the clamp plate 1603 is slidably positioned into a first, open flow position 1611, permitting the tubing set 1600 to pass unrestricted through the clearance portion in the clamp profile 1605, and the flow within the tubing set being constrained only by the constraint profiles within the first and second constraint members 1601 and 1602. In one or more embodiments, when the position of the clamp plate 1603 is in the open flow position 1611, one or more clamp state indicators 1607 is provided to a user corresponding to the state of the clamp, such as the word “open,” “ready,” “go,” “run,” or another suitable term.

FIG. 16C shows a constraint assembly 1613 comprising first constraint member 1601 and 1602 assembled to constrain a tubing set 1600, the constraint members also capturing a slidable clamp plate 1603 with a tubing set 1600 passing through clamp profile 1605. In one or more embodiments the clamp plate 1603 is slidably positioned into a second, closed flow position 1612, fully collapsing one or more medication lumens disposed within the tubing set 1600 within the narrow portion of the clamp profile 1605, thus preventing medication flow. In one or more embodiments when the position of the clamp plate 1603 is in the closed flow position 1612, one or more clamp state indicators 1608 is provided to a user corresponding to the state of the clamp, such as the word “closed,” “stop,” “pause,” or another suitable term.

In one or more embodiments, the clamp profile 1605 is designed so that in the open position, the tubing set 1600 may freely pass through the clamp profile 1605 with no interference or further reduction in fluid flow, except as provided by the constraint profiles of the assembled constraint members 1601 and 1602. In one or more embodiments, the clamp profile 1605 comprises a rapidly narrowing design, whereby movement of the clamp profile 1605 from the open 1611 position to a second closed position 1612 quickly halts flow through the tubing set 1600. In one or more embodiments, the clamp profile 1605 is matched to the cross section of the unconstrained tubing set 1600. In one or more embodiments, the clamp is supplied to a user after manufacture in the open flow position 1611, allowing immediate medication administration at the constrained rate provided by the assembly 1610. In one or more embodiments, the clamp is supplied to a user after manufacture in the closed flow position 1612, preventing medication administration until the clamp plate 1603 is slidably moved to an open flow position 1611, thereafter allowing immediate medication administration at the constrained rate provided by the assembly 1610.

As mentioned, liquid medication formulations are commonly administered from a drug delivery device, through tubing sets, where flow rate can be controlled using a pre-defined tubing set inner diameter, each corresponding to a flow rate for a given medication. In such a configuration, flow rate is controlled by the tubing set selection; the drug delivery device does not alter flow rate parameters. For instance, two tubing sets, each with different internal diameters, may be labeled for 150 mL/h or 300 mL/h when using a specific fluid, such as saline. One disadvantage of this approach is that if an intermediate flow rate is desired (e.g., 200 mL/h), another dedicated tubing set must be manufactured and/or sourced, adding cost and inefficiency associated with the delivery of medication to a patient. As such, one embodiment of the present disclosure relates to improved apparatus and methods to constrain flowrates through a tubing set while maintaining generic, mass-manufacturable tubing sets.

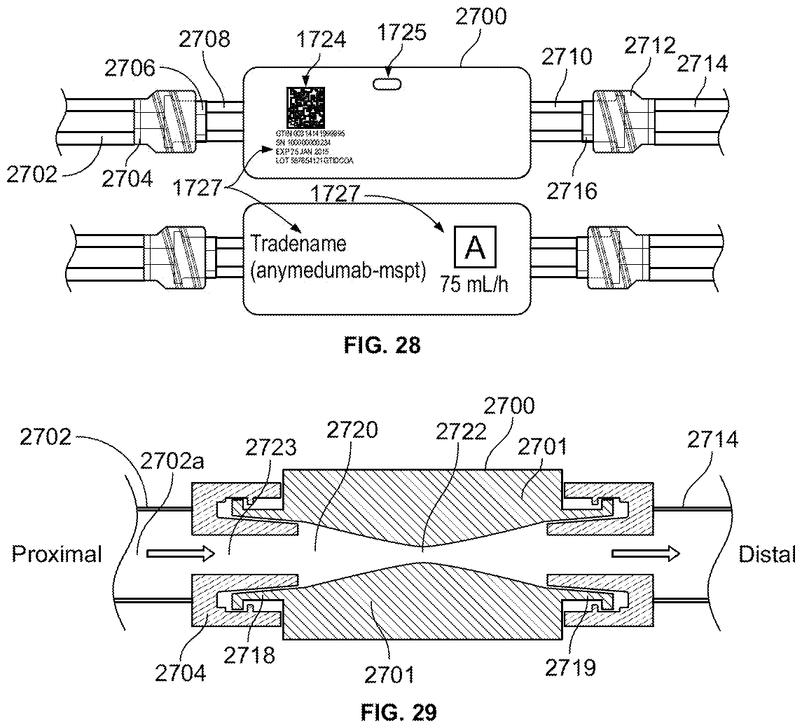

Unlike the embodiments described above where an external tubing constraint is assembled surrounding a flexible tubing set, an alternate embodiment may be preferable to minimize additional assembly steps in manufacturing and/or operation of a medicament delivery system. The alternative modular constraint assembly described below may also alleviate supply chain concerns, as the prior embodiments require separate stock keeping units (SKUs) or similar inventory keeping units for each combination of tubing and constraint. Accordingly, FIGS. 28-29 depict modular constraint assemblies 2700 that serve as a connector between two pieces of medication administration tubing 2702, 2714. In such modular constraint assemblies, there is an internal fluid lumen 2720 having a first diameter with a flow-restricting cross-sectional area 2722 having a second diameter, where the arrows in FIG. 29 depict fluid flow path. Multiples sizes of the modular constraint assemblies can be manufactured corresponding to predetermined and desired constraint internal diameters and medication flow rate.

As illustrated in FIGS. 28 and 29, the modular constraint assemblies can be designed with releasable and reusable connectors 2706, 2718 that may be interposed between two tubing segments 2702, 2714, where each tubing set segment has cooperating connectors 2704, 2716, such that, each end of the modular constraint assembly 2708, 2710 or 2718, 2719 attaches to the end of generic medication administration tubing 2702, 2714. Preferably, reusable and releasable connectors, such as, a luer-taper or Luer-Lok® fitting can be used on each end, as these are common on existing tubing sets. The connectors associated with the modular constraint assembly can be integral to single molded piece of the constraint 2701, e.g., connectors 2718, 2719 (see FIG. 29) or the connectors 2706, 2716 can be connected to the terminal ends of tubing 2708, 2710 that attached to the modular constraint 2700.

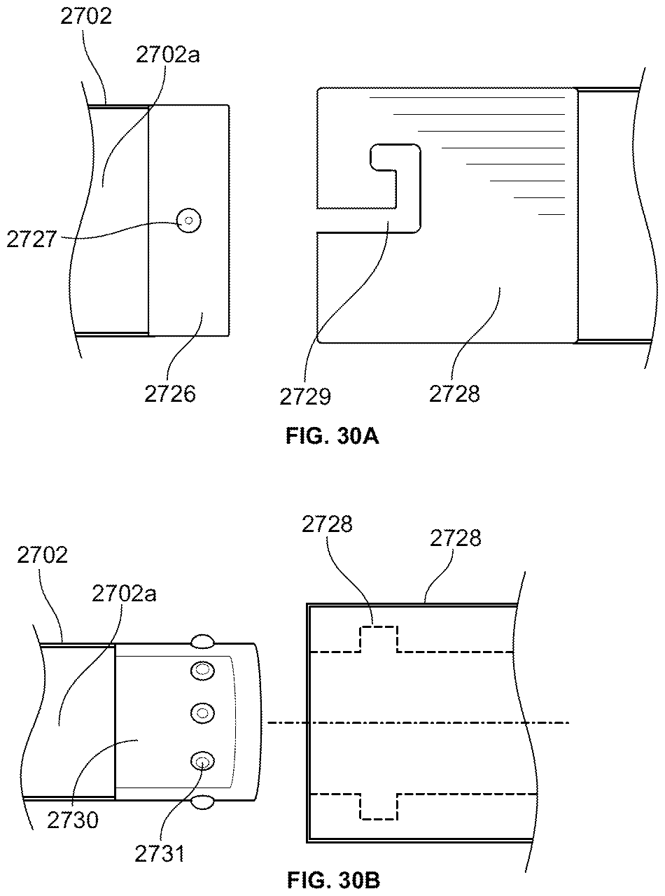

Alternatively, the connectors on the modular constraint assembly may connect to one or both of the tubing sets with a non-standard connection to prevent connection to off-the-shelf components. Such a keyed or unique proprietary design of the connectors further improves medication safety and also allows a specific tubing set to be “enforced” with a specific pump design, preventing misconnections or inadvertent substitution that are problematic with prior art devices. Two examples of such a keyed connector design are depicted in FIGS. 30A and 30 B. FIG. 30A depicts a slotted pin connection 2727, 2729 between the tubing set 2726 and a portion of the modular constraint assembly 2728. FIG. 30B depicts an alternative keyed connector design where detent connection 2731 and 2728 is present between the tubing set 2730 and a portion of the modular constraint 2728. Optionally, the connection between the constraint and the tubing sets may be non-reversible, i.e., a non-reusable and permanent connection. This may be advantageous as a tamper-proof method post assembly in manufacture or dispensing facility (e.g., pharmacy).

In some embodiments of the modular constraint assemblies of the present disclosure it may be desirable to include a sliding clamp plate, e.g., 1603 (see FIG. 16B), at either the proximal or distal end of the modular constraint assembly. The clam plate could include clamp travel tabs, e.g., 1604, to slidably engage with a clamp travel slot, e.g., 1606, provided on one of the ends of the modular constraint assembly such that tubing 2708 or 2710 extending from the end of the modular constraint assembly passes the clamp profile 1605 during assembly. Slidably engaging the clamp plate 1603 within the travel slot 1606 between the clamp travel stops allows flow in the tubing to be fully stopped or started depending on the position of the tubing within the clamp profile 1605.

The modular constraint assemblies of the present disclosure provide two unique aspects. First, the modular constraint assembly is symmetric (male-male or female-female) so that the two pieces of a tubing set cannot be connected without using a modular constraint assembly. This prevents non-use of the modular constraint assembly (and resulting free-flow conditions) or use of the medicament delivery device without the proper flow rate constraint in place. Second, the constraint 2701 is a molded, rigid component, and thus has zero compliance against pressure in the tubing set system, a significant improvement over the short tubing set segments used in prior art systems (e.g., Koru Medical fluidic restrictor). As indicated, certain medications, such as biologic medications, displaying temperature-viscosity-concentration relationships and non-Newtonian behavior, such as shear thinning or shear thickening. As such, in one or more embodiments of the modular constraint assembly, the constraint profile and length may be adjusted for each specific flow rate desired to lower the force needed for medication delivery, to provide sufficiently high flow rates, accommodate shear-thinning behavior, to accommodate shear-thickening behavior, to accommodate flow at a recommended administration temperature, to accommodate changes to concentration of a medication formulation at one or more administration doses, or to increase patient comfort. Likewise, the length of the constraint 2701 can be varied to establish a specific, calibrated flow rate based on specific characteristics of the therapeutic medications passing through the internal lumen, the specific characteristics selected from the group consisting of viscosity, shear thinning behaviors, shear thickening behaviors, desired delivery time to the patient, and combinations thereof. Each of the possible embodiments of the modular constraint assemblies of the present disclosure can have smoothed internal features to prevent damage to a medicament (e.g., protein shear) or accommodate non-Newtonian behaviors for a specific medication. Similarly, the constraint 2701 shape, contours and size may be paired to a specific medication and flow behaviors.

Many variations in construction, assembly, and design are possible for one or more embodiments of the apparatus described herein, depending on the clinical application, characteristics of the medication being delivered, tubing set design, desired flow rates of one or more medications, and pharmacokinetics of the medications once in the body.

The constraint members and clamp plates described herein can be made of, by way of example, a plastic material such as polyamide, polycarbonate, acrylonitrile butadiene styrene, polypropylene, high density polyethylene, polyester, polyoxymethylene, other polymers, combinations of polymers, or other materials having suitable rigidity and strength to withstand the assembly operation described herein. Alternatively, the constraint members described herein may be made of a composite material, such as a polymer reinforced with fiberglass, polymer reinforced with aramid fiber, polymer reinforced with carbon fiber, or another suitable composite of fiber- or metal-reinforced polymer. In one or more embodiments, the constraint members are molded from a single material. In one or more embodiments, the constraint members are molded from multiple materials, as with a two-shot or over molding process. In one or more embodiments, the clamp plate and constraint members are the same materials. In one or more embodiments, the clamp plate and constraint members are different materials. In one or more embodiments, either or both of the clamp plate and constraint members contain a slip additive to reduce sliding friction.

In one or more embodiments, the material for one or more constraint members is chosen for compatibility with gamma, e-beam, or other radiation sterilization methods. In one or more embodiments, the design and material of one or more constraint members is chosen for compatibility with gamma, e-beam, or other radiation sterilization methods. In one or more embodiments, the design of one or more constraint members is chosen to ensure the assembled constraint members retain their strength against inadvertent disassembly or further adjustment after sterilization with gamma, e-beam, or other radiation sterilization methods.

In an embodiment, the design of the locking mechanism is such that the magnitude of the force used to assemble the constraint members into each other via the locking fingers and apertures intentionally exceeds the compressive force that may be manually applied by a human without use of the constraint apparatus, thereby requiring mechanical assembly equipment and preventing intentional or inadvertent post-manufacturing adjustment of the applied constraint apparatus by a user after manufacture. In an embodiment, the design of the locking mechanism is such that the magnitude of the force to separate the assembled constraint members intentionally exceeds the tensile force that may be manually applied by a human without use of the mechanical assistance, such as with a tool or fixture, thereby preventing intentional or inadvertent removal of the applied constraint apparatus by a user after manufacture. In one or more embodiments, the design of the constraint members is configured to prevent access to either or both of the locking fingers and apertures, discouraging tampering, removal, or further adjustment by a user of the apparatus after manufacture and assembly to a tubing set.

Other suitable locking mechanisms may be substituted to secure the constraint members including press fit, ultrasonic welding, heat staking, adhesive cured by exposure to ultraviolet light after application, or other suitable adhesives. In one or more embodiments, the assembly method is selected to reduce leachable or extractable components that may be introduced from the assembled constraint members through the wall of an interposed tubing set and one or more medications flowing within one or more tubing set medication lumen(s) situated therein. In another embodiment, a selectively curable adhesive is applied to either or both of the apertures and locking fingers on either or both constraint members prior to assembly, the members are assembled, and the adhesive is cured, thus preventing further movement or inadvertent adjustment of the constraint. In one or more embodiments, the selectively curable adhesive comprises an ultraviolet-cure adhesive.

In one or more embodiments, it may be desirable to isolate the material(s) comprising the constraint members or one or more adhesives used to assemble the constraints from the tubing set and one or more medications flowing therein. Accordingly, in one or more embodiments, some or all of the contoured constraint profiles may be provided with a barrier coating interposed between the constraint profile and tubing set surface. In one or more embodiments, the barrier coating comprises a PTFE or other fluoropolymer material.

In one or more embodiments, the constraint profiles have a substantially curvilinear cross-section. In one or more alternative embodiments, the constraint profiles have a substantially linear cross-section. In one or more alternative embodiments, the one or more contoured constraint profiles have a substantially constant cross-sectional design. In one or more alternative embodiments, the one or more contoured constraint profiles have a substantially variable cross-sectional design.

In one or more embodiments, the one or more contoured constraint profiles are configured to constrain one or more lumens situated within a tubing set equally. In one or more embodiments, the one or more contoured constraint profiles are configured to constrain one or more lumens situated within a tubing set unequally. In one or more embodiments, the one or more contoured constraint profiles are configured to constrain specific lumens situated within a tubing set, while also being configured to leave other specific lumens contained within a tubing set unconstrained. In one or more embodiments, the one or more contoured constraint profiles are configured to selectively engage with one or more medication lumens situated within a tubing set. In one or more embodiments, one or more constraint profiles are configured to selectively engage with one or more medication lumens situated within a tubing set. In one or more embodiments, one or more constraint profiles are configured to provide one or more different levels of constraint when disposed around a tubing set containing one or more medication lumens. In one or more embodiments, the one or more contoured constraint profiles are configured to constrain one or more fluid lumens in a desired manner, while also being configured to avoid avoiding constraint, crimping, or damage to an electrical or optical conductor disposed within the tubing set in a substantially parallel manner to the one or more medication lumens.

In one or more embodiments, the constraint is applied to the exterior of a tubing set that has already been sterilized. In one or more embodiments, the constraint is applied to a tubing set prior to sterilization of the assembled apparatus. In one or more embodiments, the material selection of constraint members and design of the locking fingers and apertures anticipates embrittlement or loss of strength after sterilization. In one or more embodiments, the material selection of constraint members and design of the locking fingers and apertures is designed to retain full design strength after radiation sterilization, for example by gamma or e-beam methods.

Elements of the tubing sets described herein may take various shapes and forms. In one or more embodiments, the outer surface of the tubing set may take a substantially circular or elliptical shape. The flexible portion of the tubing set may be fashioned from silicone, PVC, PVC without DEHP, EVA, HDPE, LDPE, TPU, PTFE, polyurethane, a fluoropolymer, or other suitable flexible material. In one or more embodiments, the flexible portion of the tubing set is manufactured from multiple connected segments of one or more flexible materials to provide a tubing set with different flexibility along the length. In one or more embodiments, the flexible portion of the tubing to which the constraint is applied may be manufactured from a more or less flexible material than the remainder of the tubing set.

Certain medications, such as biologic medications, displaying temperature-viscosity-concentration relationships and non-Newtonian behavior, such as shear thinning or shear thickening. In one or more embodiments, the constraint member profiles and length may be adjusted for each specific flow rate desired to lower the force needed for medication delivery, to provide sufficiently high flow rates, accommodate shear-thinning behavior, to accommodate shear-thickening behavior, to accommodate flow at a recommended administration temperature, to accommodate changes to concentration of a medication formulation at one or more administration doses, or to increase patient comfort.

In one or more embodiments, a kit of components is provided, comprising a plurality of tubing sets, each with assembled constraint apparatus applied as described herein, wherein the constraint member length and constraint profile of each applied constraint corresponds to one or more discrete desired flow rates for a specific medication at the anticipated administration temperature. In one or more embodiments, each constrained tubing set contained within the kit provides a different flow rate of a specific medication. In one or more embodiments, each constrained tubing set contained within the kit provides the same flow rate of a specific medication at one or more different administration temperatures. In one or more embodiments, each constrained tubing set contained within the kit provides the same flow rate of a specific medication at one or more different concentrations. In one or more embodiments, each constrained tubing set contained within the kit provides the same flow rate of a specific medication at one or more different concentrations at the same administration temperature. In one or more embodiments, each constrained tubing set contained within the kit provides one or more different flow rates of a specific medication at one or more different concentrations. In one or more embodiments, each constrained tubing set contained within the kit provides one or more different flow rates of a specific medication at one or more different concentrations at the same administration temperature. In some embodiments, the specific administration temperature is room temperature as governed by the ISO-1 standard. In some embodiments, the specific administration temperature is approximately 20° C.

In one or more embodiments, a kit of components is provided, comprising a plurality of tubing sets, each with assembled constraint apparatus applied as described herein, wherein the constraint member length and constraint profile of each applied constraint corresponds to one or more discrete desired flow rates for a non-Newtonian (e.g., biologic) medication at the anticipated administration temperature. In one or more embodiments, each constrained tubing set contained within the kit provides a different flow rate of a non-Newtonian (e.g., biologic) medication. In one or more embodiments, each constrained tubing set contained within the kit provides the same flow rate of a non-Newtonian (e.g., biologic) medication at one or more different administration temperatures. In one or more embodiments, each constrained tubing set contained within the kit provides the same flow rate of a non-Newtonian (e.g., biologic) medication at one or more different concentrations. In one or more embodiments, each constrained tubing set contained within the kit provides the same flow rate of a non-Newtonian (e.g., biologic) medication at one or more different concentrations at the same administration temperature. In one or more embodiments, each constrained tubing set contained within the kit provides one or more different flow rates of a non-Newtonian (e.g., biologic) medication at one or more different concentrations. In one or more embodiments, each constrained tubing set contained within the kit provides one or more different flow rates of a non-Newtonian (e.g., biologic) medication at one or more different concentrations at the same administration temperature. In some embodiments, the specific administration temperature is room temperature as governed by the ISO-1 standard. In some embodiments, the specific administration temperature is approximately 20° C.

In one or more embodiments, a kit of components is provided, comprising a plurality of tubing sets, each with assembled constraint apparatus applied as described herein, wherein the constraint member length and constraint profile of each applied constraint corresponds to one or more discrete desired flow rates for a medication studied in a human clinical trial, each of the discrete desired flow rates corresponding to one or more clinical trial test conditions for the medication being studied in the trial. In one or more embodiments, one or more of the kit, kit components, tubing sets, or tubing set constraints contains indicia related to the clinical trial, clinical trial identifier, clinical trial medication, clinical trial test condition, clinical trial randomization schedule identifier, or patient identifier.

In one or more embodiments, each constrained tubing set contained within the kit provides a different flow rate of a specific clinical trial medication under investigation. In one or more embodiments, each constrained tubing set contained within the kit provides the same flow rate of a specific clinical trial medication under investigation at one or more different administration temperatures. In one or more embodiments, each constrained tubing set contained within the kit provides the same flow rate of a specific clinical trial medication under investigation at one or more different concentrations. In one or more embodiments, each constrained tubing set contained within the kit provides the same flow rate of a specific clinical trial medication under investigation at one or more different concentrations at the same administration temperature. In one or more embodiments, each constrained tubing set contained within the kit provides one or more different flow rates of a specific clinical trial medication under investigation at one or more different concentrations. In one or more embodiments, each constrained tubing set contained within the kit provides one or more different flow rates of a specific clinical trial medication under investigation at one or more different concentrations at the same administration temperature. In some embodiments, the specific administration temperature is room temperature as governed by the ISO-1 standard. In some embodiments, the specific administration temperature is approximately 20° C.

In one or more embodiments, each clinical trial subject is assigned a kit of components comprising a plurality of tubing sets, each with assembled constraint apparatus applied as described herein, wherein the constraint member length and constraint profile of each applied constraint corresponds to one or more discrete desired flow rates for a medication studied in a human clinical trial, and wherein each of the discrete desired flow rates correspond to one or more clinical trial test conditions for the medication being studied in the trial, and wherein a clinical trial subject is administered medication through one of the sets within the kit in one or more instances. In one or more embodiments, each clinical trial participant is assigned a kit of components comprising a plurality of tubing sets, each with assembled constraint apparatus applied as described herein, wherein the clinical trial subject receives a different kit of components at each dosing interval during a clinical trial. In one or more embodiments, each clinical trial subject is assigned a kit of components comprising a plurality of tubing sets, each with assembled constraint apparatus applied as described herein, and wherein a clinical trial subject is administered medication with only one of the tubing sets within the kit. In one or more embodiments, each clinical trial subject is assigned a kit of components comprising a plurality of tubing sets, each with assembled constraint apparatus applied as described herein, and wherein a clinical trial subject is administered medication with one or more of the tubing sets within the kit. In one or more embodiments, each clinical trial subject is assigned a kit of components comprising a plurality of tubing sets, each with assembled constraint apparatus applied as described herein, and wherein a clinical trial subject is administered medication using one or more of the tubing sets within the kit based on their physiologic response to a medication studied in a human clinical trial. In one or more embodiments, one or more of the kit, kit components, tubing sets, or tubing set constraints contains indicia related to the clinical trial, clinical trial identifier, clinical trial medication, clinical trial test condition, clinical trial randomization schedule identifier, or patient identifier.

In one or more embodiments, the kit of components used in a clinical trial is provided in a commercial presentation of a medication studied in a human clinical trial once approved for general use by a regulatory approval body. In one or more embodiments, a subset of the kit of components used in a clinical trial is provided in a commercial presentation of a medication studied in a human clinical trial once approved for general use by a regulatory approval body. In one or more embodiments, multiple different kits, each containing a subset of the kit of components used in a clinical trial is provided in a commercial presentation of a medication studied in a human clinical trial once approved for general use by a regulatory approval body. In one or more embodiments, one or more of the kit, kit components, tubing sets, or tubing set constraints provided in a commercial presentation of an approved medication contains different indicia than that used in one or more clinical trials of the medication.

In addition to the aspects described herein related to constraint of one or more medications flowing through a tubing set, constraint members may also serve to identify the tubing set, providing a more intuitive experience for the user and avoiding potential medication errors caused by incorrect set selection. In an alternative embodiment illustrated in FIG. 14A, the assembled first and second constraint members comprise a series of different shapes or profiles, by way of example a substantially rectilinear shape 1401, a substantially elliptical shape 1402, a substantially circular shape 1403, or a substantially polygonal shape 1404, each corresponding to different medications or different doses of the same medication. Although FIG. 14A and the accompanying specification shows a variety of geometric profiles by way of example, it will be clear to those skilled in the art that many geometric profiles are possible, and the foregoing example shall not be construed as limiting the apparatus to one of the geometric shapes or profiles described or illustrated herein.

The constraint members may be provided in a variety of shapes and colors. In some embodiments, constraint members are molded in one or more colors associated with a pharmaceutical brand. In some embodiments, the constraint members are molded in one or more colors associated with a dose of a specific medication. In some embodiments, the first constraint member is molded in a different color than the second constraint member.

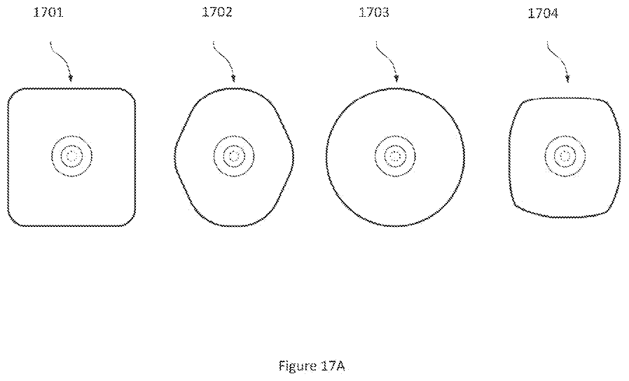

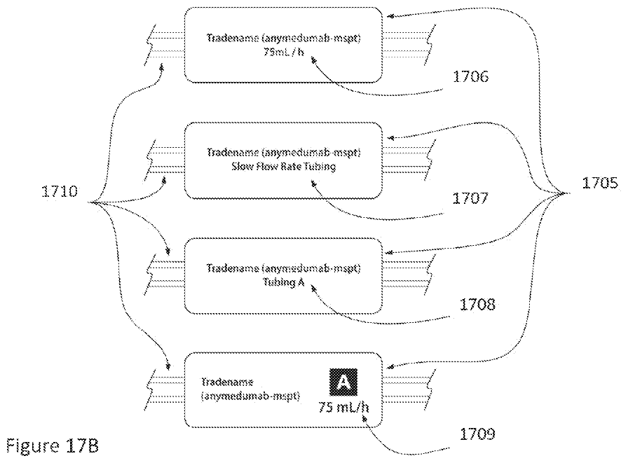

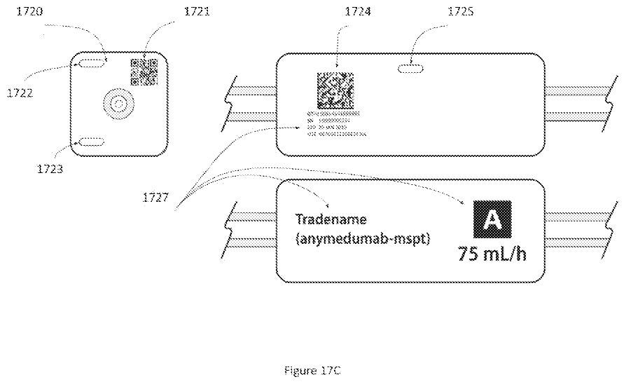

As illustrated in FIG. 17B, one or more assembled constraint members 405 may be provided with indicia related to the medication and flow rate. In another embodiment, one or more assembled constraint members 1705 may be provided with indicia 1706 related to the medication and flow rate. In another embodiment, one or more assembled constraint members 1705 may be provided with indicia 1706, 1707, 1708, 1709 related to the medication and an ordinal identifier identifying the flow rate in simple terms for a user lacking healthcare training, such as a patient. Indicia 1706, 1707, 1708, and 1709 may be applied to the constrain members by molding, co-molding of a polymeric material (optionally in a contrasting color), in-mold decoration, by laser marking, pad printing, or other means. In some embodiments, the indicia includes the specific temperature used to calibrate the flowrate noted in the indicia. In some embodiments, the indicia includes one or more warnings, cautions, or instructions to a user of the tubing set. In one or more embodiments, one or more of the kit, kit components, tubing sets, or tubing set constraints contains indicia related to the clinical trial, clinical trial identifier, clinical trial medication, clinical trial test condition, clinical trial randomization schedule identifier, or patient identifier. In one or more embodiments, one or more of the kit, kit components, tubing sets, or tubing set constraints contains indicia that disguise or “blind” a clinical participant to one or more of the medication (or a placebo medication in a randomized clinical trial), clinical trial test condition, clinical trial randomization schedule, or clinical trial sponsor.

The constraints described herein may also be used to identify the constraint elements during manufacturing or assembly, avoiding mix-ups and improper component selection. Referring to FIG. 17C, an assembled constraint 1720 may be provided with one or more machine-readable indicia 1721, 1722, 1723, 1724, and 1725. In one or more embodiments, one or more of the machine-readable indicia 1721, 1722, 1723, and 1725 comprise a bar code, near-field communication (NFC) or a radiofrequency identification (RFID) tag. In one or more embodiments, information on one or more of the machine-readable indicia 1721, 1722, 1723, 1724, and 1725 is populated during manufacturing of the apparatus. In one or more embodiments, one or more of the machine-readable indicia 1721, 1722, 1723, 1724, and 1725 are configured to be written one time with information during manufacturing and read-only thereafter. In one or more embodiments, one or more of the machine-readable indicia 1721, 1722, 1723, 1724, and 1725 are configured to be written after manufacturing.

In one or more embodiments, information on one or more of the machine-readable indicia 1721, 1722, 1723, 1724, and 1725 is populated during dispensing of the apparatus. In one or more embodiments, information on one or more of the machine-readable indicia 1721, 1722, 1723, 1724, and 1725 is populated with patient information from an electronic health record. In one or more embodiments, information on one or more of the machine-readable indicia 1721, 1722, 1723, 1724, and 1725 is populated with medication administration or monitoring instructions from an electronic health record. In one or more embodiments, information on one or more of the machine-readable indicia 1721, 1722, 1723, 1724, and 1725 is populated with either or both of physiologic or laboratory values which constitute safe medication administration parameters for the specific tubing set and assembled constraint 1720.

In one or more embodiments, one or more of the machine-readable indicia 1721, 1722, 1723, 1724, and 1725 are located on the exterior surface of the assembled constraint and/or the constraint members. In one or more embodiments, one or more of the machine-readable indicia 1721, 1722, 1723, 1724, and 1725 are located below the exterior surface of the assembled constraint. In one or more embodiments, one or more of the machine-readable indicia 1721, 1722, 1723, 1724, and 1725 are inserted into the constraint members during injection molding. In one or more embodiments, one or more of the machine-readable indicia 1721, 1722, 1723, 1724, and 1725 are applied to one or more of the constraint members or assembled constraint after molding.

In one or more embodiments, a first electronic indicia 1722 is provided on the first constraint member, and a second electronic indicia 1723 is provided on a second constraint member, both constraint members comprising an assembled constraint 1720, configured so that during manufacturing operations, the first 1722 and second 1723 electronic indicia may be verified by inspection equipment to verify the proper components are selected, the apparatus components are assembled in the correct orientation, and the apparatus matches the expected medication and flow rate. In one or more embodiments, electronic indicia 1725 is provided on the assembled constraint 1725 as a verification during manufacturing and packaging that the apparatus matches the expected medication and flow rate.

In one or more embodiments, machine readable indicia 1721, 1724 is provided on one or more exterior surfaces of the assembled constraint 1720 as a verification during manufacturing and packaging that the apparatus matches the expected medication and flow rate. In one or more embodiments, the machine-readable indicia 1721, 1724 comprises a one or more of a QR code, data matrix, 2D bar code, or linear bar code. In one or more embodiments, human-readable indicia 1727 is provided on one or more exterior surfaces of the assembled constraint 1720 as verification that the apparatus matches the expected medication and flow rate. In one or more embodiments, the information contained in the machine-readable indicia 1721, 1724 is the same as that information contained in the human-readable indicia 1727. In one or more embodiments, the information contained in the human-readable indicia 1727 is a subset of the information contained in the machine-readable indicia 1721, 1724. In one or more embodiments, the information contained in the machine-readable indicia 1721, 1724 is different from that information contained in the human-readable indicia 1727.

In one or more embodiments, one or more of the indicia 1721, 1722, 1723, 1724, 1725, or 1727 encodes one or more of tubing set outer diameter, tubing set material, tubing set material lot code, constraint material lot code, internal batch control numbers, number of fluid lumens disposed within the tubing set, tubing set medication lumen diameters, tubing set medication lumen arrangement within the tubing set cross section, number of electrical conductors disposed within the tubing set, number of optical conductors disposed within the tubing set, the medication name, the medication dose, the medication concentration, the medication lot number, the medication expiration date, the numeric medication flow rate (e.g., in mL/h) corresponding to the constraint, the ordinal identifier of the flow rate corresponding to the constraint (e.g., “slow set,” “fast set,” or “Set A”) the medication administration temperature corresponding to the flow rate, the presence or absence of a clamping device in the apparatus, tubing set apparatus lot code, tubing set apparatus serial number or unique device identifier, tubing set apparatus Global Trade Item Number (GTIN), or tubing set apparatus expiration date.

The constraints described herein may also be used to identify the constraint elements during dispensing to a patient or clinical use, avoiding mix-ups and improper component selection. In one or more embodiments, electronic indicia 1725 are provided on the assembled constraint for scanning or sensing by a drug delivery system, patient, clinician, or other user of the device to ensure proper tubing sets are used. In one or more embodiments, one or more of the kit, kit components, tubing sets, or tubing set constraints contains indicia that disguise or “blind” a clinical participant to one or more of the medication (or a placebo medication in a randomized clinical trial), clinical trial test condition, clinical trial randomization schedule, or clinical trial sponsor.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, those skilled in the art will understand that the embodiments described are merely illustrative of the principles and applications of the present disclosure. It will be apparent to those skilled in the art that various modifications and variations can be made to the method and apparatus of the present disclosure without departing from the spirit and scope of the disclosure. Thus, the present disclosure can include modifications and variations that are within the scope of the appended claims and their equivalents.

In one or more embodiments, the constraint apparatus, systems, methods and kits described immediately above with respect to FIGS. 14-17 are configured to be used or combined with the apparatus, systems and methods described with respect to FIGS. 1-10 and the numbered embodiments described with respect to FIGS. 1-10. Thus, in some embodiments, the apparatus, systems and methods described and claimed with respect to FIGS. 1-10 further comprise the constraint apparatus, systems, methods and kits described immediately above with respect to FIGS. 14-17 in combination or addition with the various embodiments described with respect to FIGS. 1 to 10, including the numbered embodiments.

Optionally, the drive system used in a medicament delivery device as described herein is a pressure-based drive. In certain pump configurations, it may be desirable to provide a pneumatic drive for one or more fluidic reservoirs. Alternatively, where a pneumatic drive is described herein, another pressure-based drive such as a hydraulic drive may be used instead. FIG. 21 shows two configurations of a system to which the present disclosure is directed, allowing for centralized (bottom) or decentralized/distributed (top) distribution of components on a patient receiving medication with the medicament delivery device. The medicament delivery device comprises a (typically reusable) device 2101 (powerpack portion), for example a housing containing a powerpack (including a pump, for example a pneumatic pump), and a (typically single use (optionally refurbishable)) cartridge unit 2102 (cassette portion). Either the device and cartridge are centralized (bottom), for example by being directly attached to one another, or decentralized/distributed (top), for example spaced apart and only attached together by an umbilical comprising, for example, a tubing set 2103, which comprises a tube with at least one lumen, and optionally comprises one or more conductors (e.g. one or more cables). In the bottom example in particular, the cartridge unit may be wearable, and may be supported separately. For completeness, the tubing 2104 between the cartridge unit 2102 and the patient 2105 is also shown—this could be multiple tubes as shown, or a single tube. A medicament delivery member such as a cannula, a needle or a jet injector can be provided at the end of the tubing 2104 distal to the cartridge unit 2102.

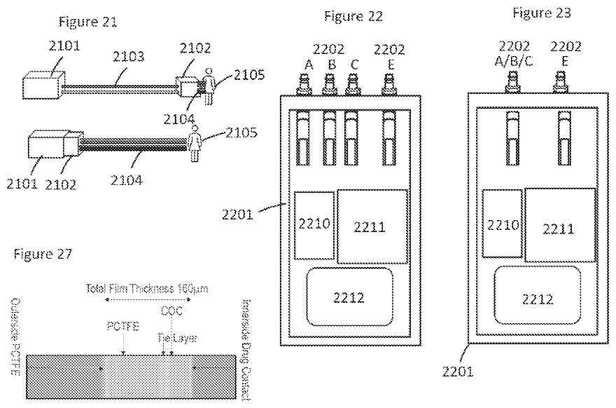

FIG. 22 depicts a device 2201; the device 2201 could be used for the device shown as device 2101 in FIG. 21. The device comprises separate valving for each medication (2202 A-C) and optionally also for an emergency medication (2202 E). The device 2201 may also comprise a power source 2210, one or more pumps 2211 and a user interface 2212. The power source could be a battery, a spring, and/or a gas canister, for example. One pump 2211 could be provided, or one pump could be provided for valves A to C and a second pump could be provided for valve E, for example. Optionally, one pump is provided for each valve. The user interface 2212 could include one or more buttons and/or a screen, for example. Each valve could be associated with a separate medication—in the example in FIG. 22, there could therefore be three medications (A-C) and an emergency medication (E).

FIG. 23 shows a similar device to that in FIG. 22, but with a single valve 2202A/B/C for multiple medications instead. This may also allow a sequential pneumatic delivery as described previously for the expected medication administration (A/B/C), and a separate air line for contingent administration of an optional emergency medication (E). Further details of administration options are outlined in more detail elsewhere in the application and will not be repeated here. In particular, one or more of the lumens in the tubing sets described elsewhere in the application could contain a fluid such as a gas (e.g. air or nitrogen or argon) or a liquid (e.g. water), rather than containing medication, with the fluid being pressurized by a pump and thereby squeezing a medication out of a container and thereby delivering the medication to a patient. Such fluids may be run through one more lumens in a multiple lumen tubing set as desired, for example based on the clinical applications as described below in representative scenarios.

Provision of pneumatic elements (i.e. a lumen or lumens providing drive elements), medicament delivery elements (i.e. a lumen or lumens providing medicament delivery elements), and/or communication elements (for example to connect monitoring systems within different parts of a system electrically and/or optically) within separate lumens of the same tube can be beneficial as it can simplify device assembly and/or device use by reducing the number of separate tubes that are required.

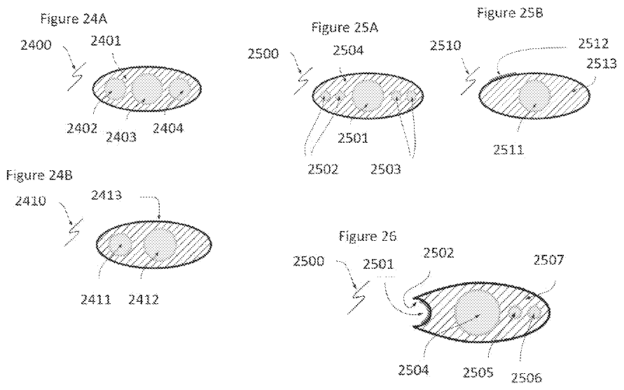

In general, a tubing set for a medicament delivery device may be provided. The tubing set comprises one or more lumens. Optionally, the tubing set comprises multiple lumens; this can be beneficial for ease of assembly and for ease of use. Optionally, one of the lumens is a medicament lumen; that is, a lumen for transmission of medicament. Optionally, one of the lumens is a pneumatic fluid lumen; in such a case, a fluid (such as a gas, e.g. air, nitrogen or argon, or a liquid such as water) would typically be pressurized by a drive, for example to drive a medicament out of a medicament container by compression of the medicament container by transfer of the pneumatic fluid into a container at least partially surrounding the medicament container. Optionally, the tubing set comprises a conductor; this could be used to transfer data from one part of the medicament delivery device to another, or to transfer commands from one part of the medicament delivery device to another. The conductor could be an electrical conductor or an optical conductor. The conductor could be in one of the lumens. Alternatively, the conductor could be embedded in the tubing set, or could be attached to an outer wall of the tubing set. Optionally, the conductor is in an undercut of the tubing set (see for example FIG. 26). This can help reduce or avoid the risk of the conductor detaching from the tubing set when the tubing set is flexed during use. This can also help provide structural support to the tubing set, particularly in the case of an electrical conductor. The tubing set has a longitudinal axis extending along the length of the tubing set (optionally in the direction of the multiple lumens, as in FIG. 12G for example), with the undercut arranged on a long cross-sectional axis, the long cross-sectional axis being the longest axis of the tubing set perpendicular to the longitudinal axis. For reference, the longitudinal axis would be in the direction of the arrows of FIG. 18D, for example, or would be into the page in FIGS. 12 and 24 to 26. A short cross-sectional axis is perpendicular to the longitudinal axis and perpendicular to the long cross-sectional axis. The long cross-sectional axis is the first axis 1240 in FIG. 12G, for example, and the short cross-sectional axis is the second axis 1241 in FIG. 12G, for example. Optionally, some or all of the lumens are arranged along the long cross-sectional axis, as in FIGS. 12G and 25A, for example. Optionally, one of the multiple lumens is arranged along a short cross-sectional axis, as in FIGS. 12, 24, 25 and 26, for example. Optionally, the tubing set is oval when viewed in cross-section perpendicular to the longitudinal axis, as in FIG. 24, for example, although other cross-sectional shapes, such as round, square, hexagonal or other regular or irregular shapes, could alternatively be used. A medicament delivery device could comprise one or more tubing sets as described in this paragraph or as described elsewhere in this application.

In one example, medicament delivery device is configured to deliver a therapeutic medication to a patient, and comprises a tubing set as described herein (for example any tubing set according to FIG. 12, 24, 25 or 26) along with a reservoir containing a therapeutic medication. The apparatus can comprise a medicament delivery member, such as a cannula, a needle or a jet injector. The apparatus can comprise at least one sensor configured to detect at least one of a physiological aspect of the patient and a physical aspect of the apparatus. The apparatus can comprise a controller configured to receive data from the sensor, and to start and stop delivery of the therapeutic medication to the patient in response to data received from the sensor. Alternatively or additionally, the apparatus can comprise a controller configured to control the injection rate of one or more medicaments. The apparatus can comprise a drive unit configured to deliver the contents of the reservoir (typically a medicament) into the patient. The drive unit can be a pressure-based drive such as a pneumatic drive unit, for example using a pressurized fluid such as pressurized gas from a gas canister or using a pump to pressurize a fluid. Other drive units, such as mechanical drives, for example using springs, or even manually driven drives could alternatively be used. Optionally, the medicament delivery device is for delivery of an oncology medicament. Optionally, the medicament delivery device is for delivery of two or more medicaments. Optionally, the medicament delivery device is for delivery of one or more medicaments and for contingent delivery of an emergency medicament, wherein the device is configured to deliver the emergency medicament to the patient if one or more pre-determined conditions are met.

A medicament delivery device can have two or more tubing sets. In one example, a first tubing set (for example tubing set 2103) connects a drive unit (such as device 2101) to a cartridge unit (such as cartridge 2102), and a second tubing set (for example tubing set 2104) connects a cartridge unit (such as cartridge 2102) to a medicament delivery site. In more detail, a configuration such as the top configuration from FIG. 21 can be used, with a pneumatic drive in the device 2101, one or more lumens in one or more tubing sets 2103 (these lumens being used for pneumatic fluid—so for pneumatic communication), one or more medicament containers in the cartridge 2102, one or more lumens in one or more tubing sets 2104 (these lumens being used for medicament—so for fluidic communication). Optionally, electrical or optical communication could also be provided in one or both of the first and second tubing sets, for example to transmit sensor data and/or to transmit commands. As mentioned previously, a medicament delivery member such as a cannula, a needle or a jet injector can be provided at the end of the tubing 2104 distal to the cartridge unit 2102 (optionally one medicament delivery member used for a plurality of lumens, or one medicament delivery member per lumen, or a combination of both).

Further examples of pneumatic, fluidic, electrical and optical communication are described below. These approaches could be used in the top configuration from FIG. 21, for example.

### Pneumatic Communication

The tubing sets described elsewhere in this application may be used for pneumatic and/or fluidic elements of the drug delivery system. FIG. 24A shows a tubing set 2400 with a cross section 2401 and multiple lumens 2402, 2403, 2404. Lumens 2402, 2303, 2404 may be configured to communicate with components of the system as described elsewhere herein and/or with components of the systems as described in 63/392,539, the full contents which is herein incorporated by reference. For instance, lumen 2403 may be configured to be in pneumatic communication with a controller and drive (e.g. air pump) and a pressurized portion of a fluid reservoir (cassette) as shown in FIG. 21, while lumens 2403-2404 may be in fluidic communication with a reservoir (a fluidic reservoir, for example a fluidic reservoir containing a medicament) as described elsewhere herein.

### Pneumatic & Fluidic Communication

FIG. 24B shows a tubing set 2410 with a cross section 2413 and multiple lumens 2411, 2412 configured to communicate with components of the drug delivery system as described elsewhere herein and/or with components of the systems as described in 63/392,539,the full contents of which is herein incorporated by reference. For instance, lumen 2411 may be configured to be in pneumatic communication with a controller and drive (e.g. air pump) and a pressurized portion of a fluid reservoir (cassette) as shown in FIG. 23 for delivery of medications in a regimen (denoted “A/B/C”) while lumen 2411 may be used to pressurize a fluid reservoir (cassette) as shown in FIG. 23 for contingent delivery of medication (e.g. emergency medications). Put another way, lumen 2411 may be used for medications delivered in the normal course of therapy, while lumen 2412 may be used for contingent administration of an emergency medication.

Pneumatic, Electrical, and/or Optical Communication

FIG. 25A shows a tubing set 2500 with a cross section 2504, lumen 2501, and conductors 2502, 2503. Lumen 2501 and conductors 2502, 2503 may be configured to communicate with components of the drug delivery system as described elsewhere herein and/or with components of the systems as described in 63/392,539, the full contents of which is herein incorporated by reference. For example, lumen 2501 may be configured to be in pneumatic communication with a controller and drive (i.e., air pump) and a pressurized portion of a fluid reservoir (cassette) as shown in FIG. 1, while conductors 2502, 2503 may be in electrical and/or optical communication (or combinations thereof) with the controller as described elsewhere herein.

FIG. 25B shows a tubing set 2510 with a cross section 2513, lumen 2511, and conductor 2512. Lumen 2511 and conductors 2512 may be configured to communicate with components of the system as described elsewhere herein and/or with components of the systems as described in 63/392,539, the full contents of which is herein incorporated by reference. For instance, lumen 2511 may be configured to be in pneumatic communication with a controller and drive (i.e., air pump) and a pressurized portion of a fluid reservoir (cassette) as shown in FIG. 21, while conductor 2512 may be in electrical communication with a controller or sensors as described elsewhere herein.

FIG. 26 shows a tubing set 2600 with a cross section 2607, lumen 2604, undercut 2602, and conductors 2605, 2606, all as described elsewhere herein. Lumen 2604 and conductors 2605, 2606 may be configured to communicate with components of the drug delivery system as described elsewhere herein and/or with components of the systems as described in 63/392,539, the full contents of which is herein incorporated by reference. For example, lumen 2604 may be configured to be in pneumatic communication with a controller and drive (i.e., air pump) and a pressurized portion of a fluid reservoir (cassette) as shown in FIG. 21, while conductors 2605, 2606 may be in electrical and/or optical communication (or combinations thereof) with the controller as described elsewhere herein.

The drug delivery devices and components described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis, cancer and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

In general in this application, unless indicated otherwise, a ‘tubing set’ or ‘tubing’ may comprise one or more tubes, each tube comprising one or more lumens.

Based on the aspects described above, the following specific examples of the present disclosure are presented. The first concerns an apparatus configured to deliver a therapeutic medication to a patient, where the apparatus comprises: a reservoir containing a therapeutic medication; a patient interface configured to deliver contents of the reservoir into the patient; and at least one sensor configured to detect at least one of a physiological aspect of the patient and a physical aspect of the apparatus; a controller configured to receive data from the sensor, and to start and stop delivery of the therapeutic medication to the patient in response to data received from the sensor; and a tubing set with at least one medication lumen having an internal surface and an external surface, the at least one medication lumen in fluid communication with the reservoir at a proximal end of the tubing set and the patient interface at a distal end of the tubing set, the tubing set also comprising at least one conductor in electrical or optical communication with the controller at the proximal end of the tubing set and the sensor at a distal end of the tubing set. At least one conductor of the apparatus is situated internally within the tubing set. The at least one conductor can be situated internally within the tubing set and said at least one conductor is substantially parallel to the at least one medication lumen from the proximal to the distal end of said tubing set. Also, the at least one conductor can be situated on an external surface of the tubing set. The at least one conductor situated on the external surface of the tubing set comprises a conductive ink.

The apparatus of the present disclosure may also comprise a barrier coating situated on the internal surface of the at least one medication lumen, the barrier coating further configured to isolate medication within the at least one medication lumen from undesirable extractable or leachable materials from either the tubing set or the at least one conductor. The barrier coating may comprise polytetrafluoroethylene. Further, the barrier coating can be interposed between the at least one conductor and the external surface of the at least one medication lumen, where the barrier coating may comprise polytetrafluoroethylene. The apparatus may further comprise a protective sheath over the at least one conductor on the external surface of the at least medication lumen. An undercut feature can be situated on an exterior contour of the tubing set of the apparatus, said undercut containing one or more electrical or optical conductors. The protective sheath may substantially enclose the undercut feature and any electrical or optical conductors situated therein.

The tubing set of the apparatus may comprise an asymmetric cross-section with an undercut feature containing one or more electrical or optical conductors, the undercut feature further being situated in the tubing set cross-section exhibiting the highest bending stiffness. The tubing set may further comprise an optical conductor, wherein the optical conductor comprises an optical fiber. Alternatively, the tubing set may comprise an optical conductor and the controller comprising at least one light disposed therein, the light configured to operate in conjunction with the optical conductor to alert a user as to an aspect or status of a drug delivery apparatus connected to the tubing set. The apparatus can also have an illuminated alert to a user of the apparatus, where the illuminated alert presented to a user can be a color code including different colors or as a pulsed pattern of one or more different colors.

The apparatus can also be configured such that the alert presented to a user of the apparatus comprises a signal as to the status of a drug delivery device connected to the tubing set, selected from one of the group of a) the drug delivery device is properly configured, b) the drug delivery device is ready to administer medication to a patient, c) the drug delivery device is currently administering one or more medications to a patient, d) the drug delivery device detected an error in configuration prior to administration, e) the drug delivery device detected an error during administration of one or more medications by the drug delivery device, f) the drug delivery device has completed medication administration, g) the drug delivery device has detected administration taking place at an unsafe rate, h) the drug delivery device has detected loss of skin contact at the patient interface, i) the drug delivery device has detected disconnection from or occlusion of the patient interface, or j) the tubing set in the apparatus is incorrect or incompatible with the medication within the drug delivery system, k) the drug delivery device has detected a suspected infusion reaction, l) the drug delivery device has detected anomalous physiologic sensor data, m) the drug delivery device has detected a tubing set occlusion, n) the patient interface is incorrectly configured, o) the drug delivery system has administered an emergency medication to a patient, p) the drug delivery system has a low battery level, q) the drug delivery system has lost connection to a sensor, r) the drug delivery system has lost connectivity to an external system or server, s) the drug delivery system has an expired medication or component, t) the drug delivery system has administered a pre-medication, u) the drug delivery system has administered a post-medication, v) the drug delivery system has lost connection to a telemedicine service, w) the drug delivery system has lost wireless connection, or x) the drug delivery system has lost cellular connection.

Some further aspects of the invention are outlined in the following clauses.

1. An apparatus configured to deliver a therapeutic medication to a patient, the apparatus comprising:
a reservoir containing one or more therapeutic medications;
a patient interface configured to deliver contents of the reservoir into the body of the patient;
a flexible tubing set in fluid communication with the reservoirs at a proximal end of the flexible tubing set, and the patient interface at a distal end of the flexible tubing set; and
a fluid pump configured to expel the therapeutic medication from the reservoirs through the flexible tubing set and into the patient interface,
wherein the flexible tubing set comprises a predetermined length and an internal lumen comprising a consistent internal diameter, the flexible tubing set configured to provide a predetermined, calibrated flow rate based on specific characteristics of the therapeutic medications passing through the internal lumen, the specific characteristics selected from the group consisting of viscosity, shear thinning behaviors, shear thickening behaviors, desired delivery time to the patient, and combinations thereof.

2. The apparatus of embodiment Error! Reference source not found., wherein the fluid pump comprises a substantially constant pressure device.

3. The apparatus of embodiment Error! Reference source not found., wherein the fluid pump comprises a substantially constant flow device.

4. The apparatus of any of embodiments Error! Reference source not found. to 3, wherein the internal diameter of the internal lumen is configured to reduce stresses at an interface of the medication-tubing set and associated aggregation of a protein-based therapeutic medication.

5. The apparatus of any of embodiments Error! Reference source not found. to 4, wherein the therapeutic medication is a substantially non-Newtonian fluid.

6. The apparatus of any of embodiments Error! Reference source not found. to 5, wherein the therapeutic medication exhibits a non-linear relationship between viscosity and shear stress.

7. The apparatus of any of embodiments Error! Reference source not found. to 6, wherein the therapeutic medication exhibits non-linear viscosity changes based on temperature of the medication.

8. The apparatus of any of embodiments Error! Reference source not found. to 7, wherein the therapeutic medication is a biologic, recombinant therapeutic protein, gene therapy, monoclonal antibody, antibody-drug conjugate, or fusion protein.

9. The apparatus of any of embodiments Error! Reference source not found. to 8, wherein the fluid pump is disposable and designed for one-time use.

10. The apparatus of any of embodiments Error! Reference source not found. to 8, wherein the fluid pump is reusable and designed for multiple-time use.

11. The apparatus of any of embodiments Error! Reference source not found. to 8, wherein the fluid pump is reusable and designed for use over a course of a single cycle of a medication regimen.

12. The apparatus of any of embodiments 1 to 11, further comprising a controller that is reusable and designed for use over the course of a single cycle of a medication regimen.

13. The apparatus of any of embodiments 1 to 11, further comprising a controller that is disposable and designed for one-time use.

14. The apparatus of any of embodiments 1 to 11, further comprising a controller that is reusable and designed for multiple-time use.

15. The apparatus of any of embodiments Error! Reference source not found. to 14, wherein the reservoir is administered by the fluid pump only after elapse of a pre-determined time delay.

16. The apparatus of any of embodiments Error! Reference source not found. to 15, wherein the flexible tubing set is configured to provide a flow rate less than a flow rate at which a therapeutic medication may cause an infusion reaction.

17. The apparatus of any of embodiments Error! Reference source not found. to 16, further comprising a plurality of flexible tubing sets, and wherein one or more the flexible tubing sets is labeled with an actual flow rate in mL/hour of the therapeutic medication at room temperature based on an experimentally determined concentration-temperature-viscosity relationship.

18. An apparatus configured to deliver one or more therapeutic medications to a patient, the apparatus comprising:
a plurality of reservoirs, each containing one or more therapeutic medications;
a patient interface configured to deliver contents of the reservoirs into a body of the patient;
a flexible tubing set in fluid communication with the reservoirs at a proximal end of the flexible tubing, and the patient interface at a distal end; and
a fluid pump to expel the therapeutic medication from the reservoirs through the flexible tubing set and into the patient interface,
wherein the flexible tubing set is provided with a predetermined length and internal lumen comprising a consistent internal diameter configured to provide a predetermined, calibrated flow rate based on specific characteristics of the therapeutic medications passing therethrough, the specific characteristics selected from the group consisting of viscosity, shear thinning behaviors, shear thickening behaviors, desired delivery time to the patient, and combinations thereof.

19. The apparatus of embodiment 18, wherein the fluid pump comprises a substantially constant pressure device.

20. The apparatus of embodiment 18, wherein the fluid pump comprises a substantially constant flow device.

21. The apparatus of any of embodiments 18 to 20, wherein the internal diameter of the internal lumen is configured to reduce stresses at the medication-tubing set interface and associated aggregation of a protein-based therapeutic medication.

22. The apparatus of any of embodiments 18 to 21, wherein the therapeutic medication is a substantially non-Newtonian fluid.

23. The apparatus of any of embodiments 18 to 22, wherein the therapeutic medication exhibits non-linear relationship between viscosity and shear stress.

24. The apparatus of any of embodiments 18 to 23, wherein one of the therapeutic medications exhibit non-linear viscosity changes based on temperature of the medication.

25. The apparatus of any of embodiments 18 to 24, wherein the therapeutic medication is a biologic, recombinant therapeutic protein, gene therapy, monoclonal antibody, antibody-drug conjugate, or fusion protein.

26. The apparatus of any of embodiments 18 to 25, wherein the fluid pump is disposable and designed for one-time use.

27. The apparatus of any of embodiments 18 to 25, wherein the fluid pump is reusable and designed for multiple-time use.

28. The apparatus of any of embodiments 18 to 27, wherein the controller is reusable and designed for use over the course of a single cycle of a medication regimen.

29. The apparatus of any of embodiments 18 to 27, wherein the controller is disposable and designed for one-time use.

30. The apparatus of any of embodiments 18 to 27, wherein the controller is reusable and designed for multiple-time use.

31. The apparatus of any of embodiments 18 to 25 and 28 to 30, wherein the fluid pump is reusable and designed for use over the course of a single cycle of a medication regimen.

32. The apparatus of any of embodiments 18 to 31, wherein the reservoir is administered by the fluid pump only after elapse of a pre-determined time delay.

33. The apparatus of any of embodiments 18 to 32, wherein the flexible tubing set is configured to provide a flow rate less than a flow rate at which one or more therapeutic medications may cause an infusion reaction.

34. The apparatus of any of embodiments 18 to 33, wherein fluid communication between one or more reservoirs and the proximal end of the flexible tubing set is provided by a manifold.

35. The apparatus of any of embodiments 18 to 34, wherein fluid communication between one or more the reservoirs and the proximal end of the flexible tubing set comprises at two or more independent medication lumens, and wherein the diameter of at least a first and second medication lumens are substantially unequal.

36. The apparatus of any of embodiments 18 to 35, wherein fluid communication between one or more the reservoirs and the proximal end of the flexible tubing set comprises at two or more independent medication lumens, and wherein the diameter of at least a first and second medication lumens are substantially equal.

37. The apparatus of any of embodiments 18 to 36, wherein administration of the therapeutic medication from each of the plurality of reservoirs occurs in a predetermined order.

38. The apparatus of any of embodiments 18 to 37, wherein the therapeutic medication from a first of each of the plurality of reservoirs is administered by the fluid pump only after elapse of a pre-determined time delay.

39. The apparatus of any of embodiments 18 to 38, wherein the therapeutic medication from one or more of each of the plurality of reservoirs is administered by the fluid pump only after of a pre-determined time delay that is substantially equal for each of the plurality of reservoirs.

40. The apparatus of any of embodiments 18 to 38, wherein the therapeutic medication from each of the plurality of reservoirs is administered by the fluid pump only after elapse of a pre-determined time delay that is substantially different for each of the plurality of reservoirs.

41. The apparatus of any of embodiments 18 to 40, wherein the therapeutic medications from the plurality of reservoirs are concurrently administered to a patient by the fluid pump.

42. The apparatus of any of embodiments 18 to 40, wherein the therapeutic medications from the plurality of reservoirs are sequentially administered to a patient by the fluid pump, and wherein administration of the therapeutic medication from each of the reservoirs begins only after administration of the therapeutic medication from a preceding reservoir is completed.

43. The apparatus of any of embodiments 18 to 40, wherein the therapeutic medications from the plurality of reservoirs are sequentially administered to a patient by the fluid pump, and wherein administration of the therapeutic medication from a subsequent reservoir begins only after administration of the therapeutic medication from a preceding reservoir begins.

44. The apparatus of any of embodiments 18 to 40, wherein the therapeutic medications from the plurality of reservoirs are sequentially administered to a patient by the fluid pump, and wherein beginning of administration of the therapeutic medication from each of the plurality of reservoirs is separated by one or more time delays.

45. The apparatus of any of embodiments 18 to 44, further comprising a plurality of flexible tubing sets and wherein one or more the flexible tubing sets is labeled with the actual flow rate in mL/hour of the therapeutic medication at room temperature based on an experimentally determined concentration-temperature-viscosity relationship.

46. The apparatus of any of embodiments 18 to 45, further comprising a plurality of flexible tubing sets and wherein one or more the flexible tubing sets is labeled with an ordinal identifier corresponding to one or more of the flow rates of the therapeutic medication at room temperature based on an experimentally determined concentration-temperature-viscosity relationship.

47. The apparatus of any of embodiments 18 to 46, wherein the fluid pump comprises a substantially constant pressure device.

48. The apparatus of any of embodiments 18 to 46, wherein the fluid pump comprises a substantially constant flow device.

49. The apparatus of any of embodiments 18 to 48, wherein the flexible tubing sets provides flow rates less than the rate at which the therapeutic medication may cause an infusion reaction.

50. The apparatus of any of embodiments 18 to 49, further comprising a plurality of flexible tubing sets and wherein the plurality of tubing sets are selected from between two and ten different configurations of predetermined lengths and internal lumen of consistent internal diameters.

51. An apparatus configured to deliver a therapeutic medication to a patient, the comprising:
one or more reservoirs, each of the one or more reservoirs containing one or more therapeutic medications;
one or more reservoirs containing either or both of a pre-medication to be administered before or a post-medication to be administered after the one or more therapeutic medications;
a patient interface configured to deliver contents of the reservoirs into the body of the patient;
a flexible tubing set in fluid communication with the reservoirs at a proximal end of the flexible tubing set, and a patient interface at a distal end of the flexible tubing set; and
a fluid pump to expel the therapeutic medication from each of the one or more reservoirs through the flexible tubing set and into the patient interface,
wherein the flexible tubing set is provided with predetermined length and an internal lumen of consistent internal diameter to provide a specific, calibrated flow rate based on characteristics of the therapeutic medications passing therethrough, the characteristics selected from the group consisting of viscosity, shear thinning behaviors, shear thickening behaviors, desired delivery time to the patient, and combinations thereof.

52. The apparatus of embodiment 51, wherein one or more the pre-medications or the post-medications are selected from the group consisting of an analgesic, an antipyretic, a corticosteroid, an antihistamine, an antiemetic, an antithrombotic, or an antimicrobial.

53. The apparatus of embodiment 51, wherein one or more of the pre-medications or the post-medications comprise a co-formulated antimicrobial and antithrombotic.

54. The apparatus of embodiment 51, wherein one or more the pre-medications or the post-medications are selected from the group consisting of diphenhydramine, acetaminophen, ondansetron, famotidine, hydrocortisone, dexamethasone, and methylprednisolone.

55. The apparatus of embodiment 51, wherein one or more the pre-medications or the post-medications are selected from the group consisting of 0.9% Normal Saline, Heparin Lock Flush solution, 100 U/mL Heparin Lock Flush Solution, or 5000 U/mL Heparin Lock Flush Solution.

56. The apparatus of embodiment 51, wherein one or more the pre-medications or the post-medications is recombinant tissue plasminogen activator (r-TPA).

57. The apparatus of embodiment 51, wherein one or more the post-medications is epinephrine.

58. The apparatus of embodiment 51, wherein one or more the pre-medications is an animal derived, human-derived, or recombinant hyaluronidase enzyme.

59. The apparatus of any of embodiments 51 to 58, wherein fluid communication between one or more the reservoirs and the proximal end of the flexible tubing set comprises two or more independent medication lumens, and wherein a first medication lumen is used to administer a therapeutic medication, and a second medication lumen is used to administer either or both of the pre-medications and post-medications.

60. The apparatus of any of embodiments 51 to 59, wherein administration of the therapeutic medication from each of the one or more reservoirs occurs in a predetermined order.

61. The apparatus of any of embodiments 51 to 60, wherein the therapeutic medication from a first of each of the one or more reservoirs is administered by the fluid pump only after elapse of a pre-determined time delay.

62. The apparatus of any of embodiments 51 to 61, wherein the apparatus is configured to administer one or more pre-medications, followed by administration of one or more therapeutic medications after a pre-determined time delay.

63. The apparatus of any of embodiments 51 to 62, wherein the apparatus is configured to administer one or therapeutic medications, followed by administration of one or more post-medications medications after a pre-determined time delay.

64. The apparatus of any of embodiments 51 to 63, wherein the therapeutic medication from one or more of each of the one or more reservoirs is administered by the fluid pump only after of a pre-determined time delay that is substantially equal for each of the one or more reservoirs.

65. The apparatus of any of embodiments 51 to 63, wherein the therapeutic medication from each of the one or more reservoirs is administered by the fluid pump only after elapse of a pre-determined time delay that is substantially different for each of the one or more reservoirs.

66. The apparatus of any of embodiments 51 to 65, wherein the therapeutic medications from the one or more reservoirs are concurrently administered to a patient by the fluid pump.

67. The apparatus of any of embodiments 51 to 65, wherein the therapeutic medications from the one or more reservoirs are sequentially administered to a patient by the fluid pump, and wherein administration of the therapeutic medication from each of the reservoirs begins only after administration of the therapeutic medication from a preceding reservoir is completed.

68. The apparatus of any of embodiments 51 to 65, wherein the therapeutic medications from the one or more reservoirs are sequentially administered to a patient by the fluid pump, and wherein administration of the therapeutic medication from a subsequent reservoir begins only after administration of the therapeutic medication from a preceding reservoir begins.

69. The apparatus of any of embodiments 51 to 65, wherein the therapeutic medications from the one or more reservoirs are sequentially administered to a patient by the fluid pump, and wherein beginning of administration of the therapeutic medication from each of the one or more reservoirs is separated by one or more time-delays.

70. An apparatus configured to deliver a therapeutic medication to a patient, the apparatus comprising:
one or more reservoirs, each of the one or more reservoirs containing a therapeutic medication;
an emergency reservoir containing an emergency medication;
a patient interface configured to deliver contents of the reservoirs into the body of the patient;
a flexible tubing set in fluid communication with the reservoirs at a proximal end of the flexible tubing set, and a patient interface at a distal end of the flexible tubing set; and
a fluid pump to expel the therapeutic medication from each of the one or more reservoirs through the flexible tubing set and into the patient interface,
at least one sensor configured in communication with the controller to detect at least one of a physiological aspect of the patient and a physical aspect of the therapeutic medication delivery apparatus;
a controller having a memory, the controller configured to receive data from the sensor, and to control one or more of start, stop, slow, speed, or continue delivery of the therapeutic medication to the patient in response to data received from the sensor; and
wherein the flexible tubing set is provided with predetermined length and internal lumen of consistent internal diameter to provide a specific, calibrated flow rate based on characteristics of the therapeutic medications passing therethrough, the characteristics selected from the group consisting of viscosity, shear thinning behaviors, shear thickening behaviors, desired delivery time to the patient, and combinations thereof.

71. The apparatus of embodiment 70, wherein the fluid pump is in communication with a controller, the apparatus thereby configured to halt administration of a therapeutic medication based upon sensor data received by the controller.

72. The apparatus of embodiment 70 or 71, wherein the fluid pump is in communication with a controller, the apparatus thereby configured to halt administration of a therapeutic medication based upon a patient's self-assessment communicated to the controller.

73. The apparatus of any of embodiments 70 to 72, wherein the fluid pump is in communication with a controller, the apparatus thereby configured to begin administration of an emergency medication based upon sensor data received by the controller.

74. The apparatus of any of embodiments 70 to 73, wherein the fluid pump is in communication with a controller, the apparatus thereby configured to begin administration of a therapeutic medication based upon a patient's self-assessment communicated to the controller.

75. The apparatus of any of embodiments 70 to 74, wherein fluid communication between one or more of the reservoirs and the proximal end of the flexible tubing set comprises at least two or more independent medication lumens, a first medication lumen being used to deliver one or more therapeutic medications by the fluid pump, and a second medication lumen being used to administer an emergency medication with the fluid pump if directed by the controller.

76. The apparatus of any of embodiments 70 to 75, wherein the controller is also configured to compare one or more sensor values to a database of sensor values held in the controller memory, the database values representing either of variously safe and unsafe medication administration conditions.

77. The apparatus of embodiment 76, wherein the controller is also configured to halt the fluid pump when an unsafe administration condition is detected by the controller and the one or more sensors.

78. The apparatus of any of embodiments 70 to 77, wherein the controller is also configured to prevent the fluid pump from administering one or more therapeutic medications when an unsafe administration condition is detected by the controller and the one or more sensors.

79. The apparatus of embodiment 78, wherein the controller is also configured to notify a healthcare provider when an unsafe administration condition is detected in a patient using the apparatus by the controller and the one or more sensors.

80. The apparatus of any of embodiments 70 to 79, wherein the controller is also configured to detect onset of an infusion reaction in a patient using the apparatus by the controller and the one or more sensors.

81. The apparatus of embodiment 80, wherein the controller is also configured to notify a healthcare provider when the onset of an infusion reaction is detected in a patient using the apparatus by the controller and the one or more sensors.

82. The apparatus of any of embodiments 70 to 81, wherein the controller is also configured to allow a healthcare provider to remotely start, stop, pause, speed, slow, or continue medication administration when an unsafe administration condition is detected in a patient using the apparatus by the controller and the one or more sensors.

83. The apparatus of any of embodiments 70 to 82, wherein the controller is also configured to allow a healthcare provider to remotely start, stop, pause, speed, slow, or continue medication administration when an infusion reaction is detected in a patient using the apparatus by the controller and the one or more sensors.

84. The apparatus of any of embodiments 70 to 83, wherein the controller is also configured to compare one or more sensor values to one or more sensor values held in the controller memory, the sensor values held in the controller memory representing physiologic data previously associated with imminent or actual infusion reactions to a therapeutic medication.

85. The apparatus of any of embodiments 70 to 84, wherein the controller is also configured to compare one or more sensor values to one or more comparator values held in the controller memory, said comparator values comprising sensor data collected from one or more prior users of the apparatus before, during, or after administration of one or more of the therapeutic medications.

86. The apparatus of embodiment 85, wherein at least one of the comparator values is determined from comprise sensor values from prior administrations of a therapeutic medication to the patient currently receiving one or more medications with the apparatus.

87. The apparatus of embodiment 85 or 86, wherein at least one of the comparator values comprise sensor values from one or more participants in one or more previous human clinical trials conducted with one or more of the therapeutic medications.

88. The apparatus of any of embodiments 70 to 87, wherein the controller is also configured to compare one or more sensor values to one or more sensor values held in the controller memory, the sensor values held in the controller memory representing one or more values contained in an electronic health record.

89. The apparatus of any of embodiments 70 to 88, wherein the controller is also configured to compare one or more sensor values to one or more sensor values held in the controller memory, the sensor values held in the controller memory representing one or more values contained in a medication order for a patient currently receiving one or more medications with the apparatus.

90. The apparatus of any of embodiments 70 to 89, wherein the controller is also configured to compare one or more sensor values to one or more sensor values held in the controller memory, the sensor values held in the controller memory representing one or more values contained in a medication order set for a patient currently receiving one or more medications with the apparatus.

91. The apparatus of any of embodiments 70 to 90, wherein the controller is also configured to prevent the fluid pump from administering one or more therapeutic medications when one or more patient laboratory values are unavailable or outside of safe administration values.

92. The apparatus of any of embodiments 70 to 91, wherein the controller is also configured to prevent the fluid pump from administering one or more therapeutic medications when one or more requisite precursor medications have not been administered to a patient.

93. The apparatus of any of embodiments 70 to 92, further comprising an interface to an electronic health record system.

94. The apparatus of any of embodiments 70 to 93, further comprising an output module interface with an electronic health record, the interface allowing update of a patient's electronic health record with one or more aspects related to delivery of one or more therapeutic medicines.

95. The apparatus of any of embodiments 70 to 94, further comprising an output module interface with an electronic health record, the interface allowing update of a patient's electronic health record with one or more aspects related to delivery of one or more emergency medicines.

96. The apparatus of any of embodiments 93 to 95, wherein the fluid pump is in communication with a controller, the apparatus thereby configured to begin administration of an emergency medication based upon sensor data received by the controller and a contingent medication order for administration of said emergency medication, said order being contained in an electronic health record system.

97. A method for using the apparatus of any of embodiments 70 to 96, comprising:
collecting sensor data from a patient before administration of a therapeutic medication to that patient using the apparatus;
identifying, via the sensor data processed by the controller, a current state of the patient prior to medication administration;
comparing, using the controller and associated computer software, one or more sensor data to one or more predetermined threshold values representing safe medication administration conditions; and
starting one of administration of a therapeutic medication to a patient if the one or more sensor data are within the one or more predefined threshold values, and preventing administration of a therapeutic medication to a patient if the one or more sensor data are outside the one or more predefined threshold values.

98. A method for using the apparatus of embodiment 97, further comprising notifying a healthcare provider as to the state of the apparatus as determined by the controller, and further comprising the healthcare provider either accepting or overriding a recommendation as to medication administration as determined by the controller.

99. A method for using the apparatus of embodiment 97 or 98, further comprising providing an alert to a user of the apparatus as to the safety of medication administration as determined by the controller.

100. A method for using the apparatus of any of embodiments 97 to 99, further comprising comparing one or more sensor values to predetermined threshold values derived from one or more prior human clinical trials of a therapeutic medication.

101. A method for using the apparatus of any of embodiments 97 to 100, further comprising comparing one or more sensor values to predetermined threshold values derived from one or more prior human clinical trials of a therapeutic medication in conjunction with the apparatus.

102. A method for using the apparatus of any of embodiments 97 to 101, further comprising comparing one or more sensor values to predetermined threshold values derived from one or more prior administrations of a therapeutic medication to a patient presently using the apparatus.

103. A method for using the apparatus of any of embodiments 97 to 102, further comprising comparing one or more sensor values to predetermined threshold values aggregated from prior administrations of a therapeutic medication to one or more patients who have previously received medication with the apparatus.

104. A method for using the apparatus of any of embodiments 70 to 96, comprising:
administering all or part of a dose of a therapeutic medication to a patient using the apparatus; and
identifying, via sensor data processed by the controller, a current state of the patient during medication administration; and
comparing, using the controller and associated computer software, one or more sensor data to one or more predetermined threshold values representing safe medication administration conditions; and
continuing administration of a therapeutic medication to a patient if the one or more sensor data are within the one or more predefined threshold values, or halting administration of a therapeutic medication to a patient if the one or more sensor data are outside the one or more predefined threshold values.

105. The method of embodiment 104, further comprising slowing an administration rate of a therapeutic medication to a patient.

106. The method of embodiment 104 or 105, further comprising providing an alert to a user of the apparatus as to a status of medication delivery.

107. The method of any of embodiments 104 to 106, further comprising providing an alert to a healthcare provider as to a status of medication delivery using the apparatus.

108. The method of any of embodiments 104 to 107, further comprising updating a patient's electronic health record with a status of medication delivery using the apparatus.

109. A method for using the apparatus of any of embodiments 70 to 96, comprising:
Administering all or part of a dose of a therapeutic medication to a patient using the apparatus; and
Identifying, via sensor data processed by the controller, a current state of the patient during medication administration; and
Comparing, using the controller and associated computer software, one or more sensor data to one or more predetermined threshold values indicative of an infusion reaction to the medication; and
Continuing administration of a therapeutic medication to a patient if the one or more sensor data do not indicate an infusion reaction is taking place; or
Halting administration of a therapeutic medication to a patient if the one or more sensor data indicate an infusion reaction is taking place.

110. The method of embodiment 109, further comprising administering an emergency medication when administration of a therapeutic medication is halted.

111. The method of embodiment 109 or 110, further comprising providing an alert to a user of the apparatus as to a status of medication delivery.

112. The method of any of embodiments 109 to 111, further comprising providing an alert to a healthcare provider as to a status of medication delivery using the apparatus.

113. The method of any of embodiments 109 to 112, further comprising updating a patient's electronic health record with a status of medication delivery using the apparatus.

114. An apparatus configured to deliver one or more investigational medicines during a clinical trial at one or more controlled flow rates, the apparatus comprising:
one or more reservoirs, each of the one or more reservoirs containing an investigational therapeutic medication;
a patient interface configured to deliver contents of the reservoirs into the body of the patient;
a flexible tubing set in fluid communication with the reservoirs at a proximal end of the flexible tubing set, and a patient interface at a distal end of the flexible tubing set; and
a fluid pump to expel the one or more investigational therapeutic medications from each of the one or more reservoirs through the flexible tubing set and into the patient interface,
at least one sensor in communication with the controller and configured to detect at least one of a physiological aspect of the patient and a physical aspect of the apparatus;
a controller configured to receive data from the sensor, and to one or more of start, stop, slow, speed, or continue delivery of the therapeutic medication to the patient in response to data received from the sensor; and
wherein the flexible tubing set is provided with predetermined length and internal lumen of consistent internal diameter to provide a specific, calibrated flow rate based on characteristics of the therapeutic medications passing therethrough, the characteristics selected from the group consisting of viscosity, shear thinning behaviors, shear thickening behaviors, desired delivery time to the patient, and combinations thereof.

115. The apparatus of embodiment 114, further comprising a one or more flexible tubing sets, each corresponding to one or more flow rates, the flow rates corresponding to one or more clinical trial conditions.

116. The apparatus of embodiment 114 or 115, the controller further comprising an interface to a clinical trial data management system.

117. The apparatus of any of embodiments 114 to 116, the controller further configured to update clinical trial data management system with a status of at least one of a physiological aspect of the patient and a physical aspect of the apparatus.

118. The apparatus of embodiment 117, the controller further configured to update clinical trial data management system with a status of at least one of a physiological aspect of the patient and a physical aspect of the apparatus before, during, and after administration of an investigational therapeutic medication.

119. The apparatus of any of embodiments 114 to 118, the controller further configured to receive information from a clinical trial data management system as to the clinical trial condition for a patient using the apparatus.

120. The apparatus of embodiment 119, the controller further configured to verify that the investigational therapeutic medication and tubing set in the apparatus are correct based on the clinical trial condition before beginning administration of the investigational therapeutic medication.

121. The apparatus of embodiment 120, the controller further configured to prevent administration of an investigational therapeutic medication if either of the investigational therapeutic medication or tubing set in the apparatus are incorrect.

122. The apparatus of any of embodiments 114 to 121, wherein the selected flexible tubing set corresponding to an individual patient's clinical trial condition is preassembled to the fluid pump.

123. A method of providing an optimized tubing set for delivery to a patient a therapeutic medication exhibiting substantially non-Newtonian characteristics delivered by a single pump unit at one or more known, preselected, and controlled flow rates, the method comprising:
identifying one or more desired flow rates of the therapeutic medication for administration to a patient based on desired pharmacokinetics of the therapeutic medication; and
identifying one or more temperatures at which delivery of the therapeutic medication will occur;
applying an adjustable constraint to a tubing set with one or more medication lumens situated therein;
compressing the constraint and tubing interposed therein to a first position;
instilling the therapeutic medication through an inlet of the tubing set so constrained, at the one or more temperatures at which delivery of the therapeutic medication will occur;
measuring the flow rate at an outlet of a tubing set so constrained;
comparing the flow rate at the outlet to the desired flow rate in the tubing;
compressing the constraint and tubing interposed therein further beyond the first position to a second position if a tested flow rate at the outlet is less than the desired flow rate, or experimentally determining a required fluid pump power to dispense the therapeutic medication if the tested flow rate at the outlet is equal to the desired flow rate; and
conducting testing to identify a relationship between temperature, viscosity, and concentration of the therapeutic medication in a pharmaceutical formulation for delivery to the patient.

124. The method of embodiment 123, wherein the therapeutic medication is a dilatant or shear-thickening fluid.

125. The method of embodiment 123, wherein the therapeutic medication is a pseudo-plastic or shear-thinning fluid.

126. The method of embodiment 123, wherein the therapeutic medication displays a substantially non-linear concentration-temperature-viscosity relationship.

127. An apparatus configured to deliver a therapeutic medication to a patient, the apparatus comprising:
a reservoir containing a therapeutic medication; and
a patient interface configured to deliver contents of the reservoir into the patient; and
at least one sensor configured to detect at least one of a physiological aspect of the patient and a physical aspect of the apparatus;
a controller configured to receive data from the sensor, and to start and stop delivery of the therapeutic medication to the patient in response to data received from the sensor; and
a tubing set with at least one medication lumen having an internal surface and an external surface, the at least one medication lumen in fluid communication with the reservoir at a proximal end of the tubing set and the patient interface at a distal end of the tubing set, the tubing set also comprising at least one conductor in electrical or optical communication with the controller at the proximal end of the tubing set and the sensor at a distal end of the tubing set.

128 The apparatus of embodiment 127, wherein the at least one conductor is situated internally within the tubing set.

129 The apparatus of embodiment 127, wherein the at least one conductor is situated internally within the tubing set, and said at least one conductor is substantially parallel to the at least one medication lumen from the proximal to the distal end of said tubing set.

130 The apparatus of embodiment 127, wherein the at least one conductor is situated on an external surface of the tubing set.

131 The apparatus of embodiment 130, wherein the at least one conductor situated on the external surface of the tubing set comprises a conductive ink.

132 The apparatus of any of embodiments 127 to 131, further comprising a barrier coating situated on the internal surface of the at least one medication lumen, the barrier coating further configured to isolate medication within the at least one medication lumen from undesirable extractable or leachable materials from either the tubing set or the at least one conductor.

133 The apparatus of embodiment 132, wherein the barrier coating comprises polytetrafluoroethylene.

134 The apparatus of any of embodiments 127 to 133, further comprising a barrier coating interposed between the at least one conductor and the external surface of the at least one medication lumen.

135 The apparatus of embodiment 134, wherein the barrier coating comprises polytetrafluoroethylene.

136 The apparatus of any of embodiments 127 to 135, further comprising a protective sheath over the at least one conductor on the external surface of the at least medication lumen.

137 The apparatus of embodiment 136, wherein the protective sheath comprises polytetrafluoroethylene.

138 The apparatus of any of embodiments 127 to 137, further comprising an undercut feature situated on an exterior contour of the tubing set, said undercut containing one or more electrical or optical conductors.

139 The apparatus of embodiment 138, further comprising a protective sheath substantially enclosing the undercut feature and any electrical or optical conductors situated therein.

140 The apparatus of any of embodiments 127 to 139, wherein the tubing set comprises an asymmetric cross-section with an undercut feature containing one or more electrical or optical conductors, the undercut feature further being situated in the tubing set cross-section exhibiting the highest bending stiffness.

141 The apparatus of any of embodiments 127 to 140, the tubing set further comprising an optical conductor, wherein the optical conductor comprises an optical fiber.

142 The apparatus of any of embodiments 127 to 141, the tubing set further comprising an optical conductor and the controller comprising at least one light disposed therein, the light configured to operate in conjunction with the optical conductor to alert a user as to an aspect or status of a drug delivery apparatus connected to the tubing set.

143 The apparatus of embodiment 142, further comprising an illuminated alert to a user of the apparatus.

144 The apparatus of embodiment 142 or 143, further comprising an illuminated alert presented to a user as a color code including different colors.

145 The apparatus of any of embodiments 142 to 144, further comprising an illuminated alert presented to a user as a pulsed pattern of one or more different colors.

146 The apparatus of any of embodiments 142 to 145, wherein the alert presented to a user of the apparatus comprises a signal as to the status of a drug delivery device connected to the tubing set, selected from one of the group of a) the drug delivery device is properly configured, b) the drug delivery device is ready to administer medication to a patient, c) the drug delivery device is currently administering one or more medications to a patient, d) the drug delivery device detected an error in configuration prior to administration, e) the drug delivery device detected an error during administration of one or more medications by the drug delivery device, f) the drug delivery device has completed medication administration, g) the drug delivery device has detected administration taking place at an unsafe rate, h) the drug delivery device has detected loss of skin contact at the patient interface, i) the drug delivery device has detected disconnection from or occlusion of the patient interface, or j) the tubing set in the apparatus is incorrect or incompatible with the medication within the drug delivery system, k) the drug delivery device has detected a suspected infusion reaction, l) the drug delivery device has detected anomalous physiologic sensor data, m) the drug delivery device has detected a tubing set occlusion, n) the patient interface is incorrectly configured, o) the drug delivery system has administered an emergency medication to a patient, p) the drug delivery system has a low battery level, q) the drug delivery system has lost connection to a sensor, r) the drug delivery system has lost connectivity to an external system or server, s) the drug delivery system has an expired medication or component, t) the drug delivery system has administered a pre-medication, u) the drug delivery system has administered a post-medication, v) the drug delivery system has lost connection to a telemedicine service, w) the drug delivery system has lost wireless connection, or x) the drug delivery system has lost cellular connection.

147 A method of delivering a therapeutic medication to a patient using the apparatus of any of embodiments 127 to 146, the method comprising:
providing electrical power from the controller to a sensor through a conductor in the tubing set; and
transmitting to the controller data obtained from a sensor; and
identifying, via sensor data processed by the controller, a current state of the patient; and
assessing, with the controller and associated computer software and/or processes, safety of continued medication administration to the patient; and
stopping, starting, slowing, speeding, or continuing flow of a therapeutic medication to a patient in response to said assessment of the safety of continued medication administration to a patient.

148 A method of delivering a therapeutic medication to a patient using the apparatus of any of embodiments 127 to 146, the method comprising:
providing electrical power from the controller to a sensor through a conductor in the tubing set; and
transmitting to the controller data obtained from a sensor; and
identifying, via sensor data processed by the controller, a current state of the patient; and
detecting, with the controller and associated computer software and/or processes, whether the patient is undergoing a systemic infusion reaction; and
stopping flow of a therapeutic medication to a patient in response if a systemic infusion reaction is detected.

149 The method of embodiment 148, wherein the method further comprises beginning administration of an emergency medication if a systemic infusion reaction is detected.

150 The method of embodiment 148 or 149, wherein the method further comprises administering an emergency through a different medication lumen than that used to administer the therapeutic medication.

151 A method of delivering a therapeutic medication to a patient using the apparatus of any of embodiments 142 to 146, the method comprising:
identifying, via sensor data processed by the controller, a current state of the patient; and
assessing, with the controller and associated computer software, a state comprising one or more aspects of the apparatus; and
providing a visual alert to the user of the apparatus corresponding to an identified state of the drug apparatus.

152 The method of embodiment 151, further comprising alerting the user with a visual feedback signal comprising one or more different colors.

153 The method of embodiment 151 or 152, further comprising alerting the user with a visual feedback signal comprising a pulsed pattern of one or more different colors.

154 A constraint apparatus for a tubing set for medication delivery comprising:
a first constraint member comprising a first constraint profile, a first aperture, and a first locking finger; and
a second constraint member comprising a second constraint profile, a second aperture, and a second locking finger, wherein the first constraint member and the second constraint member, when assembled with a tubing set disposed between the members, are configured to cooperate to constrain flow within one or more internal medication lumens contained within the tubing set, and the cooperation between the first and second constraint members provides a plurality of locking positions, each the locking position providing a different degree of constraint upon the tubing set and the one or more internal medication lumens contained within the tubing set, and wherein each degree of constraint corresponds to a predetermined and calibrated flow rate for a fluid medication at a specific concentration and a medication administration temperature.

155 The constraint apparatus of embodiment 154, wherein the fluid medication is a non-Newtonian fluid.

156 The constraint apparatus of embodiment 154 or 155, wherein the first and second members are configured to be assembled through a compressive force.

157 The constraint apparatus of embodiment 156, wherein the compressive force to assemble the first and second members in one or more of the plurality of positions exceeds the compressive force that can be manually applied by a human being without using mechanical assistance or fixturing.

158 The constraint apparatus of any of embodiments 154 to 157, wherein a tensile force to separate the first and second members in one or more of the plurality of positions exceeds the tensile force that can be manually applied by a human being without using mechanical assistance or fixturing.

159 The constraint apparatus of any of embodiments 154 to 158, wherein a compressive force to assemble the first and second members in each successive increment of constraint on the tubing set is substantially equal.

160 The constraint apparatus of any of embodiments 154 to 158, wherein a compressive force to assemble the first and second members in each successive increment of constraint on the tubing set increases with each successive increment.

161 The constraint apparatus of any of embodiments 154 to 160, wherein the constraint profiles constrain all medication lumens disposed within the tubing set interposed between the constraint members.

162 The constraint apparatus of any of embodiments 154 to 160, wherein the first and second constraint profiles constrain one or more medication lumens disposed within the tubing set interposed between the constraint members.

163 The constraint apparatus of any of embodiments 154 to 160, wherein the constraint profiles constrain one or more medication lumens disposed within the tubing set interposed between the constraint members, and wherein the constraint profiles provide substantially no constraint upon one or more medication lumens disposed within the tubing set interposed between the constraint members.

164 The constraint apparatus of any of embodiments 154 to 160, wherein the constraint profiles constrain one or more medication lumens disposed within the tubing set interposed between the constraint members, and wherein the constraint profiles provide substantially no constraint upon one or more optical or electrical conductors disposed within the tubing set interposed between the constraint members.

165 The constraint apparatus of any of embodiments 154 to 164, wherein the constraint profiles on the first and second constraint members form a substantially symmetric constraint profile upon the tubing set interposed between the first and second constraint members after assembly.

166 The constraint apparatus of any of embodiments 154 to 164, wherein the constraint profiles on the first and second constraint members form a substantially asymmetric constraint profile upon the tubing set interposed between the first and second constraint members after assembly.

167 The constraint apparatus of any of embodiments 154 to 166, wherein the constraint profiles on the first and second constraint members are specific to a single administration condition of a medication.

168 The constraint apparatus of any of embodiments 154 to 167, also comprising a clamping feature slidably engaging with the assembled first and second member between two positions, and the tubing set passing through the slidable clamping feature, the clamping feature configured to either fully stop flow in a first position, or allow flow at the rate corresponding to the constraint apparatus in a second position.

169 The constraint apparatus of embodiment 168, wherein the clamping feature engages with a portion of the tubing set not in contact with either of the constraint profiles.

170 The constraint apparatus of any of embodiments 154 to 169, wherein either of the first or second members are provided with one or more indicia related to use or manufacture of the apparatus, comprising one or more of tubing set outer diameter, tubing set material, tubing set material lot code, constraint material lot code, internal batch control numbers, number of fluid lumens disposed within the tubing set, tubing set medication lumen diameters, tubing set medication lumen arrangement within the tubing set cross section, number of electrical conductors disposed within the tubing set, number of optical conductors disposed within the tubing set, the medication name, the medication dose, the medication concentration, the medication lot number, the medication expiration date, the numeric medication flow rate (e.g., in mL/h) corresponding to the constraint, the ordinal identifier of the flow rate corresponding to the constraint (e.g., “slow set,” “fast set,” or “Set A”) the medication administration temperature corresponding to the flow rate, the presence or absence of a clamping device in the apparatus, tubing set apparatus lot code, tubing set apparatus serial number or unique device identifier, tubing set apparatus Global Trade Item Number (GTIN), or tubing set apparatus expiration date.

171 The constraint apparatus of embodiment 170, wherein the indicia comprise a machine-readable encoding.

172 The constraint apparatus of embodiment 170 or 171, wherein the indicia comprise a human readable encoding.

173 The constraint apparatus of any of embodiments 170 to 172, wherein the indicia comprises a near-field communication or radiofrequency identification chip.

174 The constraint apparatus of any of embodiments 170 to 173, wherein the indicia comprises human readable text or a scannable QR code.

175 The constraint apparatus of any of embodiments 170 to 174, wherein the indicia comprises an ordinal identifier.

176 The constraint apparatus of any of embodiments 154 to 175, the constraint apparatus, when assembled, further comprising a plurality of shapes, the plurality of shapes corresponding to a different dose of medication.

177 The constraint apparatus of any of embodiments 154 to 176, the assembled constraint apparatus also comprising a plurality of colors, the plurality of shapes corresponding to a different dose of medication.

178 A method for manufacturing a constraint apparatus, the method comprising:
orienting a first constraint member and second constraint member to align one or more locking features disposed therein or thereupon;
interposing a tubing set between the first constraint and second constraint members;
applying an increment of a compressive force to advance the locking features to a first predetermined position and to constrain the tubing set;
flowing fluid through at an inlet of the tubing set when the locking features are in the first predetermined position and measuring a flow rate at an outlet of the tubing set to obtain a measured flow rate;
comparing the measured flow rate at an outlet of the tubing set to a desired flow rate in the tubing set; and
applying an additional increment of compressive force to advance the locking features and constrain the tubing set to a more constrained, second predetermined position if the measured flow rate at an outlet of the tubing set is less than the desired flow rate, or completing the manufacturing of the constraint apparatus if the measured flow rate at an outlet of the tubing set is equal to the desired flow rate.

179 A kit comprising:
a plurality of constrained tubing sets, each constrained tubing set including an assembled constraint apparatus according to any of embodiments 154-177, wherein the constraint member constraint profile of each assembled constraint apparatus corresponds to one or more discrete desired flow rates for a specific medication at an anticipated administration temperature.

180 The kit according to embodiment 179, each constrained tubing set contained within the kit providing a different flow rate of a specific medication.

181 The kit according to embodiment 179 or 180, wherein the specific medication is a non-Newtonian fluid.

182 The kit according to any of embodiments 179 to 181, wherein the specific medication administration temperature is room temperature (20° C.).

183 The kit according to any of embodiments 179 to 182, wherein the constraint member length and constraint profile of each applied constraint corresponds to one or more discrete desired flow rates for a medication studied in a human clinical trial, each of the discrete desired flow rates corresponding to one or more clinical trial test conditions for the medication being studied in the trial.

184 The kit according to any of embodiments 179 to 183, wherein each constrained tubing set contained within the kit provides at least one of. the same flow rate of a specific medication at one or more different administration temperatures; the same flow rate of a specific medication at one or more different concentrations; the same flow rate of a specific medication at one or more different concentrations at the same administration temperature; one or more different flow rates of a specific medication at one or more different concentrations; and one or more different flow rates of a specific medication at one or more different concentrations at the same administration temperature.

185 A tubing set for a medicament delivery device, the tubing set comprising one or more lumens.

186 The tubing set of embodiment 185, wherein the tubing set comprises multiple lumens.

187 The tubing set of embodiment 186, wherein one of the multiple lumens is a medicament lumen.

188 The tubing set of embodiment 186 or 187, wherein one of the multiple lumens is a pneumatic fluid lumen.

189 The tubing set of any of embodiments 186 to 188, wherein the tubing set comprises a conductor.

190 The tubing set of embodiment 189, wherein the conductor is an electrical conductor or an optical conductor.

191 The tubing set of embodiment 189 or 190, wherein the conductor is in one of the multiple lumens.

192 The tubing set of any of embodiments 189 to 191, wherein the conductor is on an outer wall of the tubing set.

193 The tubing set of any of embodiments 189 to 192, wherein the conductor is in an undercut of the tubing set.

194 The tubing set of embodiment 193, wherein the tubing set has a longitudinal axis extending along the length of the tubing set, and wherein the undercut is arranged on a long cross-sectional axis, the long cross-sectional axis being the longest axis of the tubing set perpendicular to the longitudinal axis.

195 The tubing set of any of embodiments 185 to 194, wherein the tubing set has a longitudinal axis as defined in embodiment 194, wherein some or all of the multiple lumens are arranged along the long cross-sectional axis

196 The tubing set of any of embodiments 185 to 195, wherein one of the multiple lumens is arranged along a short cross-sectional axis, the short cross-sectional axis being perpendicular to the longitudinal axis and perpendicular to the long cross-sectional axis as defined in embodiment 194.

197 The tubing set of any of embodiments 185 to 196, wherein the tubing set is oval when viewed in cross-section perpendicular to the longitudinal axis.

198 A medicament delivery device configured to deliver a therapeutic medication to a patient, the apparatus comprising:
a tubing set according to any of embodiments 185 to 197; and
a reservoir containing a therapeutic medication.

199 The medicament delivery device of embodiment 198, the apparatus comprising a medicament delivery member.

200 The medicament delivery device of embodiment 199, wherein the medicament delivery member is a needle.

201 The medicament delivery device of embodiment 199 or 200, wherein the medicament delivery member is attached to the tubing set.

202 The medicament delivery device of any of embodiments 199 to 201, comprising:
at least one sensor configured to detect at least one of a physiological aspect of the patient and a physical aspect of the apparatus; and
a controller configured to receive data from the sensor, and to start and stop delivery of the therapeutic medication to the patient in response to data received from the sensor.

203 The medicament delivery device of any of embodiments 199 to 202, the medicament delivery device comprising a drive unit configured to deliver contents of the reservoir into the patient.

204 The medicament delivery device of embodiment 203, wherein the drive unit is a pneumatic drive unit.

205 The medicament delivery device of any of embodiments 199 to 204, wherein the medicament delivery device is for delivery of an oncology medicament.

206 The medicament delivery device of any of embodiments 199 to 205, wherein the medicament delivery device is for delivery of two or more medicaments.

207 The medicament delivery device of any of embodiments 199 to 206, wherein the medicament delivery device is for delivery of one or more medicaments and for contingent delivery of an emergency medicament, wherein the device is configured to deliver the emergency medicament to the patient if one or more pre-determined conditions are met.

208 A medicament delivery device comprising the apparatus/tubing set/kit/constraint apparatus as described in any of the embodiments above, or a medicament delivery device configured to carry out any of the methods in any of the embodiments above.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A method for determining or predicting a drug infusion reaction event comprising:
providing an infusion reaction system comprising:
a reservoir containing a therapeutic medication;
a patient interface configured to deliver contents of the reservoir into the body of a patient;
a flexible tubing set in fluid communication with the reservoir, where a proximal end of the flexible tubing set comprises the device patient interface;
a medicament drive mechanism in fluid communication with the reservoir and with the tubing set to expel the therapeutic medication into the patient interface,
a controller comprising read/write memory containing a treatment algorithm, where the controller is operatively connected to the medicament drive mechanism to control one or more of start, stop, slow, speed, or continue delivery of the therapeutic medication to the patient;
a sensor operatively connected to a patient, where the sensor is in communication with the controller to transfer sensor data collected by the sensor comprising a physiological aspect of the patient, where the controller uses the sensor data to control a physical aspect of the medicament drive mechanism;
a remote data source comprising patient anatomic and physiologic data, patient signs, symptoms, self-reports, drug delivery device data, and extracted drug, disease, and patient specific data;
collecting sensor data from the patient before administration of a therapeutic medication to that patient using the infusion reaction system;
identifying, via the sensor data processed by the controller, a current state of the patient prior to therapeutic medication administration;
comparing, using the controller and the treatment algorithm, the sensor data to a predetermined threshold value representing safe medication administration conditions; and
starting administration of the therapeutic medication to the patient if the sensor data is within the predefined threshold value or preventing administration of the therapeutic medication to the patient if the sensor data is outside the one or more predefined threshold value.

2. The method of claim 1 where the infusion reaction system further comprising a second reservoir containing an emergency medication, where the controller is operatively connected to the second reservoir such that the emergency medication can be administered to the patient through the tubing set.

3. The method of claim 1 where the medicament drive mechanism comprises a fluid pump that delivers fluid from the reservoir at flow rates determined and set by the controller.

4. The method of claim 1 further comprising notifying a healthcare provider as to the state of the infusion reaction system as determined by the controller, and further comprising providing the healthcare provider to either accept or override a recommendation as to medication administration as determined by the controller.

5. The method of claim 1 further comprising providing an alert to a user of the infusion reaction system as to the safety of medication administration as determined by the controller.

6. The method of claim 1 further comprising where the controller compares one or more sensor values to predetermined threshold values derived from one or more prior human clinical trials of a therapeutic medication.

7. The method of claim 1 further comprising where the controller compares one or more sensor values to predetermined threshold values derived from one or more prior human clinical trials of a therapeutic medication in conjunction with the infusion reaction system.

8. The method of claim 1 further comprising where the controller compares the infusion reaction system to predetermined threshold values aggregated from prior administrations of a therapeutic medication to one or more patients who have previously received medication with the apparatus.

9. The method of claim 1 further comprising:
administering all or part of a dose of the therapeutic medication to the patient using the infusion reaction system;
identifying, via the sensor data processed by the controller, a current state of the patient during medication administration;
comparing, using the controller and the treatment algorithm, the sensor data to one or more predetermined threshold values representing safe medication administration conditions; and
continuing administration of the therapeutic medication to the patient if the sensor data are within the one or more predefined threshold values, halting administration of the therapeutic medication to the patient if the sensor data is outside the one or more predefined threshold values or administering from a second reservoir emergency medication to the patient through the tubing set.

10. A method for administering a therapeutic medication and determining or predicting a drug infusion reaction event comprising:
providing an infusion reaction system comprising:
a reservoir containing the therapeutic medication;
a patient interface configured to deliver contents of the reservoir into the body of a patient;
a flexible tubing set in fluid communication with the reservoir, where a proximal end of the flexible tubing set comprises the device patient interface;
a medicament drive mechanism in fluid communication with the reservoir and with the tubing set to expel the therapeutic medication into the patient interface,
a controller comprising read/write memory containing an infusion reaction prediction algorithm, an infusion reaction typing algorithm, and an infusion reaction detection algorithm, where the controller is operatively connected to the medicament drive mechanism to control one or more of start, stop, slow, speed, or continue delivery of the therapeutic medication to the patient;
a sensor operatively connected to a patient, where the sensor is in communication with the controller to transfer sensor data collected by the sensor comprising a physiological aspect of the patient, where the controller uses the sensor data to control a physical aspect of the medicament drive mechanism;
a remote data source comprising patient anatomic and physiologic data, patient signs, symptoms, self-reports, drug delivery device data, and extracted drug, disease, and patient specific data;
executing the infusion reaction prediction algorithm to achieve infusion reaction incidence risk factor using the sensor data from the patient before administration of a therapeutic medication to that patient using the infusion reaction system and using data collected by the controller from the remote data source;
comparing the infusion reaction incidence risk factor to a predetermined risk factor to determine whether to continue with the administration of the therapeutic medication;
executing the infusion reaction typing algorithm using the sensor data, the remote data source and medicament drive mechanism data collected by the controller to generate an infusion reaction subtype; and
begin administration of the therapeutic medication through the tubing set;
executing the infusion reaction detection algorithm using the sensor data, the remote data source, the medicament drive mechanism data and the infusion reaction subtype to generate an infusion reaction determination.

11. The method of claim 10, wherein the infusion reaction determination is compared to an infusion reaction response threshold, where when the infusion reaction determination equals or exceeds the infusion reaction response threshold, then the controller will communicate with the medicament drive mechanism to control the one or more of start, stop, slow, speed, or continue delivery of the therapeutic medication to the patient.

12. The method of claim 1 where the infusion reaction system further comprising a second reservoir containing an emergency medication, where the controller is operatively connected to the second reservoir such that the emergency medication can be administered to the patient through the tubing set.

13. The method of claim 1 where the medicament drive mechanism comprises a fluid pump that delivers fluid from the reservoir at flow rates determined and set by the controller.

14. The method of claim 1 further comprising notifying a healthcare provider as to the state of the infusion reaction system as determined by the controller, and further comprising providing the healthcare provider to either accept or override a recommendation as to medication administration as determined by the controller.

15. The method of claim 1 further comprising providing an alert to a user of the infusion reaction system as to the safety of medication administration as determined by the controller.
